(19) United States
(12) Patent Application Publication
(10) Pub. No.: US 2011/0097274 A1
(43) Pub. Date: Apr. 28, 2011

(54) Carbon-11 and fluorine-18 labeled radioligands for positron emission tomography (PET) imaging for the brain serotonin transporters

(76) Inventors: Yiyun Huang, Scarsdale, NY (US); Marc Laruelle, Scarsdale, NY (US)

(21) Appl. No.: 10/439,677

(22) Filed: May 16, 2003

Related U.S. Application Data
(60) Provisional application No. 60/381,283, filed on May 17, 2002.

(51) Int. Cl. A61K 49/10; C07C 211/49; A61P 43/00

(52) U.S. Cl. 424/9.3; 564/336

(57) ABSTRACT

This invention provides a compound having the structure:

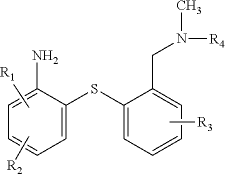

This invention also provides for related compounds and pharmaceutical compositions. This invention further provides for a compound that can be used for non-invasive method for positron emission tomography (PET) imaging of serotonin transporter sites in mammals comprising labeling serotonin transporter sites (SERT) with an image-generating amount of the radiolabeled compound disclosed herein and measuring spatial distribution of the compound in the mammal by PET so as to thereby image the serotonin transporter sites.

This application claims priority of U.S. Provisional Application No. 60/381,283, filed May 17, 2002, the contents of which are hereby incorporated by reference into this application.

This invention was made with funding from the United States Public Health Service, Grant No. NIMH K02 MH01603-0, NIMH MH59342-01, NIAAA IP50 AA-1287001. Accordingly the United States Government may have certain rights to this invention.

Throughout this application, various publications are referenced by author name and date in parentheses. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein.

### BACKGROUND OF THE INVENTION

Alterations of serotonin (5-HT) transmission have been implicated in a wide variety of psychiatric conditions, such as mood and anxiety disorders. The 5-HT transporter (SERT) is a critical protein for the regulation of 5-HT function and is the target of most commonly used antidepressants. Because of their localization on the 5-HT nerve terminals, the density of these sites is a marker for the number or integrity of the 5-HT terminals. Therefore, in vivo imaging of SERT with Positron Emission Tomography (PET) or Single Photon Emission Computerized Tomography (SPECT) provides a unique tool to study 5-HT function in health and disease.

Several postmortem studies have documented the distribution of SERT cells in the human brain (Cortes et al., 1988; Laruelle et al., 1988a; Backstrom et al., 1989; Plenge et al., 1990; Rosel et al., 1997). High SERT density is observed at the level of the midbrain nuclei, reflecting the presence of SERT on both the 5-HT cell body (raphe nuclei) and on terminals of the dense 5-HT innervation of the adjacent structures (substantia nigra, nucleus interpeduncularis, locus coeruleus, nucleus nervi hypoglossi, nucleus nervi facialis). High SERT density is also observed in the thalamus (especially in the superior and inferior colliculi and the midline nuclei, with lower density in the anterior nuclei of the thalamus), the hypothalamus and the striatum. Intermediate levels are found in the limbic and paralimbic structures, including the hippocampus, parahippocampal gyrus, entorhinal cortex, amygdala and cingulate cortex. In humans, lower levels of SERT are observed in the neocortex, and very low to negligible levels have been reported in the cerebellum.

The development of radioligands for in vivo imaging of SERT has been difficult. A large number of radioligands such as [¹¹C]cyanoimipramine, [¹¹C]sertraline, [¹¹C]dapoxetine, [¹¹C]fluoxetine, [¹¹C]citalopram, cis-[¹¹C]DDPI, [¹¹C]LY257327, [¹¹C]venlafaxine, [¹¹C]nor-beta-CIT, [¹²³I]5-iodo-6-nitroquipazine and [⁷⁶Br]5-bromo-6-nitroquipazine have been evaluated as potential PET or SPECT radioligands and found to be unsuitable due to high lipophilicity, and/or high nonspecific binding (Hashimoto et al., 1987; Lasne et al., 1989; Dannals et al., 1990; Hume et al., 1991; Nelson et al., 1991; Livni et al., 1994; Scheffel et al., 1994; Jagust et al., 1996; Bergstrom et al., 1997; Smith et al., 1997; Zea-Ponce et al., 1997; Lundkvist et al., 1999).

The SPECT radioligand [¹²³I]β-CIT, which exhibits similar affinity for dopamine transporters (DAT) and SERT (Neumeyer et al., 1991), was the first radioligand successfully used to image SERT in the living human brain. [¹²³I]β-CIT labels predominantly DAT in the striatum and SERT in the midbrain (Laruelle et al., 1993b). This regional selectivity stems from the abundance of DAT relative to SERT in the striatum, and of SERT relative to DAT in the midbrain. Thus, the midbrain uptake of [¹²³I]β-CIT has been studied in several pathological conditions (Tiihonen et al., 1997; Heinz et al., 1998; Malison et al., 1998; Jacobsen et al., 2000; Laruelle et al., 2000b; Willeit et al., 2000). While these studies yielded interesting findings regarding associations between low SERT midbrain density and depression, alcoholism, and impulsivity, they are limited by the fact that ^{[123}I]β-CIT enables the measurement of SERT binding only in the midbrain region.

[¹¹C]McN 5652 was the first PET radioligand successfully developed and used to image SERT density in humans (Suchiro et al., 1993b; Suchiro et al., 1993a; Szabo et al., 1995b; Szabo et al., 1995a; Szabo et al., 1999; Buck et al., 2000; Parsey et al., 2000). For example, [¹¹C]McN 5652 has been used to study 5-HT innervation in MDMA (“ecstasy”) abusers (McCann et al., 1998). However, [¹¹C]McN 5652 as a PET radioligand is associated with the following limitations (Parsey et al., 2000): 1) the brain uptake is protracted, requiring at least 120 minutes of data acquisition in humans to yield time-independent measures of SERT binding potential (BP) in regions with high SERT density, such as the midbrain; 2) the nonspecific binding is relatively high, thus precluding the reliable quantification of SERT in regions of relatively lower density such as the limbic system; and 3) the plasma free fraction is too low to be measured with accuracy using the conventional ultracentrifugation method, thus making it impossible to control for this variable in clinical studies.

More recently, a new series of SPECT SERT radioligands from the diarylsulfide class of compounds has been introduced and evaluated by Kung and colleges which include [¹²³I]IDAM (Acton et al., 1999a; Kung et al., 1999; Oya et al., 1999) and [¹²³I]ADAM (Choi et al., 2000; Oya et al., 2000; Acton et al., 2001). The C-11 labeled counterpart of [¹²³I]ADAM, [¹¹C]ADAM, has recently been reported, (Vercouillie et al., 2001). [¹²³I]ADAM has been shown to be suitable for imaging both the midbrain and cortex in baboons (Oya et al. 2000). However, the slow binding kinetics of this radioligand in vivo precludes its usefulness as a Positron Emissions Tomography (PET) radioligand. A number of compounds in this same class were developed by Wilson et al. as potential PET radioligands (Wilson et al., 1999, 2000). Among these, [¹¹C]DASB has been evaluated in rats and humans (Wilson et al., 2000, Houle et al., 2000; Ginovart et al., 2001; Meyer et al., 2001). DASB demonstrated an adequate signal-to-noise ratio and in vivo kinetics for a PET radioligand. It is uncertain whether [¹¹C]DASB can be used to detect SERT in the cortex. Halogenated naphthyl methoxy piperidine has also been used as a radioligand for mapping SERT by PET (Goodman, U.S. Pat. No. 5,919,797). Other diarylsulfide classes of compounds have been described for use as pharmaceuticals (Polivka et al., PCT Patent No. WO 97/17325 and Mehta et al., European Patent No. 0 402 097).

### SUMMARY OF THE INVENTION

This invention provides a compound having the structure:

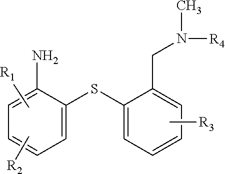

or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, (CH₂)ₙCH₃, or (CH₂)ₙCH₂X, where n=0, 1, 2, or 3;
wherein only one of R₁, R₂, and R₃ is H;
wherein X=halogen;
wherein if n=0 then (CH₂)ₙ is absent;
wherein any halogen in the compound may be a radioisotope.

This invention also provides a compound having the structure:

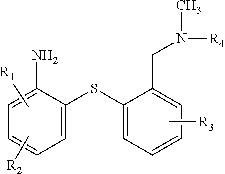

or a physiologically acceptable salt thereof;
wherein R₁ is X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, or (CH₂)ₙCH₃, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope.

This invention also provides a compound having the structure:

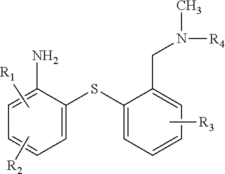

or a physiologically acceptable salt thereof;
wherein R₁ is F, (CH₂)ₙF, ¹⁸F, or (CH₂)ₙ¹⁸F, where n=1, 2, 3, or 4;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, or (CH₂)ₙCH₃, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope.

This invention also provides a physiologically acceptable composition comprising the compounds above and a physiologically acceptable carrier.

This invention also provides a process of making a physiologically acceptable composition comprising mixing the compounds above with a physiologically acceptable carrier.

This invention also provides a process for synthesizing a compound having the structure:

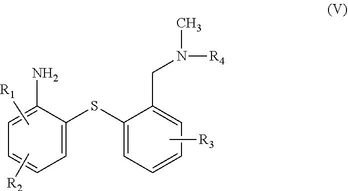

or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ⁿ(O)—, X(CH₂)ₙ(O)CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, or (CH₂)_{N}CH₃, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope,
comprising
a) reacting a compound having the structure

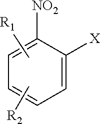

with a compound having the structure

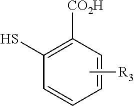

so as to form a product compound having the structure:

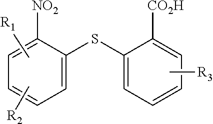

b) reacting the product compound of step a) with CH₃NHR₄ or its salt to form a product compound having the structure:

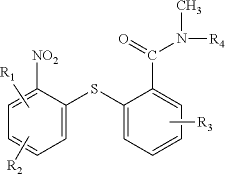

c) reacting the product compound of step b) with a reducing agent to form a product compound having the structure:

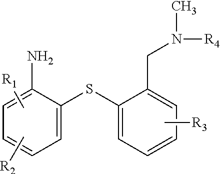

This invention also provides a process for radiolabeling with [¹¹C] a compound having the structure:

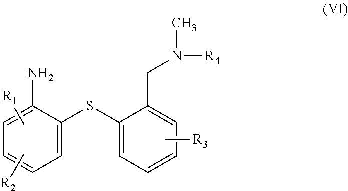

or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope,
comprising reacting the compound with [¹¹C]CH₃I in the presence of a suitable solvent.

This invention also provides a process for radiolabeling with [¹⁸F] a compound having the structure:

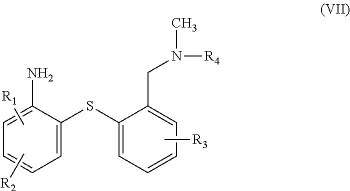

or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, (CH₂)ₙCH₃, or (CH₂)ₙCH₂X, where n=0, 1, 2, or 3;
wherein either one or two of R₁, R₂, and R₃ is H;
wherein X=halogen;
wherein if n=0 then (CH₂)ₙ is absent; and
wherein any halogen in the compound may be a radioisotope,
comprising reacting a compound with a salt of ¹⁸F⁻.

Finally this invention provides a non-invasive method for positron emission tomography (PET) imaging of serotonin transporter sites in a mammal comprising labeling serotonin transporter sites (SERT) with an image-generating amount of one of the instant radiolabeleds and measuring spatial distribution of the compound in the mammal by PET so as to thereby image the serotonin transporter sites.

### BRIEF DESCRIPTION OF THE FIGURES

FIG. 1

Chemical structures of some of the ligands evaluated in this study as imaging agents for the SERT.

FIG. 2

Mean total plasma activity normalized for injected dose [nCi/(mL*mCi ID)] following injection of [¹¹C]McN 5652, [¹¹C]ADAM, [¹¹C]DASB, [¹¹C]DAPA and [¹¹C]AFM. Each point is the mean of 4 experiments performed in two baboons. Following a rapid distribution phase, total activities stabilized at relatively constant levels. For [¹¹C]AFM and, to a lower extent, [¹¹C]ADAM, total plasma activity actually increased following the initial distribution phase, suggesting that the body distribution volume of the parent is larger than that of the metabolites.

FIGS. 3A and 3B

FIG. 3A: Mean±SD of the fraction of plasma activity corresponding to the parent compound at 2, 4, 12, 30, 60 and 90 minutes following injection of [¹¹C]McN 5652, [¹¹C]ADAM, [¹¹C]DASB, [¹¹C]DAPA and [¹¹C]AFM. [¹¹C]AFM exhibited significantly faster FIG. 3B: The symbols correspond to the mean measured values of fraction parent in the plasma. The solid lines correspond to a two-exponential fit.

FIG. 4

Coregistered MRI and PET images for the five SERT radioligands in baboon A. PET images are derived from activity collected between 40 and 90 minutes post injection. Images intensities were normalized for the injected doses. The sagittal plane shows high activities in the midbrain and thalamus, as well as low levels in the cerebellum. The transaxial plane shows the activity accumulation in the midbrain and hippocampi. Hippocampi are particularly noticeable on the [¹¹C]DASB and [¹¹C]AFM images (hippocampus is localized by an arrow on the MR image. The coronal plane is at the level of the striatum. Activity concentrations in the structures of the medial temporal lobes are again more visible on the [¹¹C]DASB and [¹¹]AFM images.

FIGS. 5A and 5B

[¹¹C]McN5652 concentrations in arterial plasma (FIG. 5A) and brain regions (FIG. 5B) following injection of 4.8 mCi of radioactivity in baboon A. Activity values were normalized to the injected dose. FIG. 5A: Plasma—Points are measured concentrations (after correction for metabolites), line represents values fitted to a sum of three exponentials. FIG. 5B: Brain—Points are activities measured in the cerebellum (open squares), temporal cortex (closed circles), striatum (open circles) and thalamus (closed squares). Lines are values fitted to a one-tissue compartment model. Axis scales were kept constant for FIGS. 5-9 to facilitate comparison between tracers.

FIGS. 6A and 6B

[¹¹C]ADAM concentrations in arterial plasma (FIG. 6A) and brain regions (FIG. 6B) following injection of 5.0 mCi of radioactivity in baboon A. Activity values were normalized to the injected dose. FIG. 6A: Plasma—Points are measured concentrations (after correction for metabolite), line represents values fitted to a sum of three exponentials. FIG. 6B: Brain—Points are activities measured in the cerebellum (open squares), temporal cortex (closed circles), striatum (open circles) and thalamus (closed squares). Lines are values fitted to a one-tissue compartment model. Axis scales were kept constant for FIGS. 5-9 to facilitate comparison between tracers.

FIGS. 7A and 7B

[¹¹C]DASB concentrations in arterial plasma (FIG. 7A) and brain regions (FIG. 7B) following injection of 5.0 mCi of radioactivity in baboon A. Activity values were normalized to the injected dose. FIG. 7A: Plasma—Points are measured concentrations (after correction for metabolite), line represents values fitted to a sum of three exponentials. FIG. 7B: Brain—Points are activities measured in the cerebellum (open squares), temporal cortex (closed circles), striatum (open circles) and thalamus (closed squares). Lines are values fitted to a one-tissue compartment model. Axis scales were kept constant for FIGS. 5-9 to facilitate comparison between tracers.

FIGS. 8A and 8B

[¹¹C]DAPA concentrations in arterial plasma (FIG. 8A) and brain regions (FIG. 8B) following injection of 3.9 mCi of radioactivity in baboon A. Activity values were normalized to the injected dose. FIG. 8A: Plasma—Points are measured concentrations (after correction for metabolite), line represents values fitted to a sum of three exponentials. FIG. 8B: Brain—Points are activities measured in the cerebellum (open squares), temporal cortex (closed circles), striatum (open circles) and thalamus (closed squares). Lines are values fitted to a one-tissue compartment model. Axis scales were kept constant for FIGS. 5-9 to facilitate comparison between tracers.

FIGS. 9A and 9B

[¹¹C]AFM concentrations in arterial plasma (FIG. 9A) and brain regions (FIG. 9B) following injection of 4.1 mCi of radioactivity in baboon A. Activity values were normalized to the injected dose. FIG. 9A: Plasma—Points are measured concentrations (after correction for metabolite), line represents values fitted to a sum of three exponentials. FIG. 9B: Brain—Points are activities measured in the cerebellum (open squares), temporal cortex (closed circles), striatum (open circles) and thalamus (closed squares). Lines are values fitted to a one-tissue compartment model. Axis scales were kept constant for FIGS. 5-9 to facilitate comparison between tracers.

FIG. 10

Ratios of fitted activities between thalamus and cerebellum, calculated every two min, for experiments depicted in FIGS. 5-9. [¹¹C]McN 5652, [¹¹C]ADAM, [¹¹C]DAPA and [¹¹C]AFM thalamus to cerebellum ratios increase continuously during the time-frame of the experiments. In contrast, [¹¹C]DASB reaches a state of transient equilibrium at about 50 minutes (thalamic and cerebellar activities decrease at a similar relative rate). The circles identify the times and levels at which the measured thalamus to cerebellum ratio is equal to the true equilibrium ratio (equal to V₃″+1). These values were: [¹¹C]McN 5652: 70 minutes, 1.78; [¹¹C]ADAM: 55 minutes, 1.92; [¹¹C]DASB: 32 minutes, 2.68; [¹¹C]DAPA: 80 minutes, 2.49; [¹¹C]AFM: 64 minutes, 3.10. These times provide an estimate of the point at which peak of specific binding occurs. Ratios measured before or after these times represent an under- and over-estimation of the true equilibrium ratios, respectively.

FIG. 11

Relationship between total duration of scan and estimates of V_{T}. Data-sets of shorter duration (40, 50, 60, 70, 80 minutes) were analyzed, and estimated V_{T} were expressed in percentage of the value derived with the complete data set (90 minutes). Each point is the average of four datasets and eight regions. Decreasing the duration of the experiment would result in over-estimation of V_{T} for [¹¹C]AFM and [¹¹C]McN 5652 and under-estimation of V_{T} for [¹¹C]DAPA, while reducing scanning time from 90 to 40 min would induce little biases on the V_{T} estimates for [¹¹C]DASB and [¹¹C]ADAM.

FIG. 12

Equilibrium specific to nonspecific partition coefficient (V₃″) measured with [¹¹C]McN 5652, [¹¹C]ADAM, [¹¹C]DASB, [¹¹C]DAPA and [¹¹C]AFM in the midbrain (Mid), thalamus (Tha), striatum (Str), hippocampus (Hip), temporal cortex (Tem), cingulate cortex (Cin) and occipital cortex (Occ). Each value is the mean±SD of four experiments (two experiments each performed in two baboons). Significant differences in V₃″ were observed between regions (p<0.0001), between tracers (p<0.0001) but not between baboons (p=0.75). Radioligand rank order of V₃″ values was [¹¹C]AFM>[¹¹C]DASB≈[¹¹C]DAPA>[¹¹C]ADAM≈[¹¹C]McN 5652.

FIG. 13

Regional V₃″ normalized to thalamic V₃″ measured with [¹¹C]McN 5652, [¹¹C]ADAM, [¹¹C]DASB, [¹¹C]DAPA and [¹¹C]AFM in the midbrain (Mid), striatum (Str), hippocampus (Hip), temporal cortex (Tem), cingulate cortex (Cin) and occipital cortex (Occ). Under the hypothesis that all tracers bind to the same population of sites, it is anticipated that the normalized V₃″ values be similar across tracers. This prediction was generally confirmed, with the exception that greater than expected V₃″ values were observed in the striatum for [¹¹C]McN 5652 and in the striatum, hippocampus and neocortical regions for [¹¹C]ADAM.

FIG. 14

Affinity (Kᵢ for SERT at room temperature) and lipophilicity (logP) of the four new compounds (ADAM, DASB, DAPA and AFM) evaluated in this study as candidate PET radioligands to label SERT in vivo. This figure illustrates that ADAM and DAPA are relatively similar, that the lower lipophilicity of DASB is associated with relatively lower affinity, and that AFM provides both a lower lipophilicity and a higher affinity. The predictions based on these data were generally confirmed in this study. [¹¹C]DASB and [¹¹C]AFM were the only compounds with measurable f₁ and f₂ fractions in vivo. [¹¹C]DASB, combining low affinity and low lipophilicity, displayed a fast kinetic of uptake and wash-out, and shorter scanning time required to derive distribution volumes. [¹¹C]AFM, displaying high affinity and low lipophilicity, provided the best specific to nonspecific contrasts. [¹¹C]McN 5652 is not presented in this figure, because predictions based on in vitro lipophilicity are of limited value for compounds belonging to different chemical classes.

FIGS. 15A and 15B

FIG. 15A: Regional brain activities normalized for injected doses in cerebellum (circles), midbrain (squares), striatum (triangles) and cingulated Cortex (diamonds) following injection of [¹¹C]AFM (open symbols) and [¹⁸F]AFM (closed symbols) in the same baboon. Regional time-activity curves were similar for both radioligands. FIG. 15B: Regional V3″ in midbrain (MID), thalamus (THA), striatum (STR), hippocampus (HIP), cingulated (CIN) and temporal cortex (TEM) for [¹¹C]AFM (white bars) and [¹⁸F]AFM (black bars).

FIG. 16

Coregistered MRI and PET images for [¹¹C] AFM (middle column) and [¹⁸F]AFM (right column) on the same baboon. PET images are derived from activity collected from 20 to 60 minutes post injection. Images were normalized for the injected dose, and the color scale is identical. The brain activity distribution is similar for both radioligands.

FIG. 17

Specific to nonspecific equilibrium partition coefficient (V₃″) of the three radiotracers AFM, AFA and AFE, radiolabeled with C-11 or F-18, in the midbrain, thalamus, striatum, cingulate cortex, temporal cortex, parietal cortex, and occipital cortex of an adult male baboon. Values are mean of 1 to 4 experiments.

### DETAILED DESCRIPTION OF THE INVENTION

Described in our invention is the compound of the general formula:

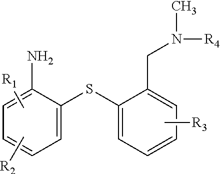

Derivatives of this compound are re-uptake inhibitors of dopamine, serotonin, and norepinephrine in the presynaptic space. Most are selective re-uptake inhibitor of serotonin and bind with high affinity to the serotonin transporter. Thus, when they are labeled with positron-emitting carbon-11 or fluorine-18, these compounds can be used to detect the concentration of serotonin transporter in the living brain using the novel imaging technique known as Positron Emission Tomography (PET).

The invention in particular relates to the preparation of imaging agents that can be used in imaging applications and investigation of serotonin transporter in psychiatric diseases such as depression, anxiety disorders and substance abuse.

This research tool can be used to non-invasively study the alterations in SERT density and 5-HT function in many neuropsychiatric disorders, and help in the development of new drugs for treatment of these disorders. Molecular imaging of the biological markers in diseases will help advance the understanding of origin and mechanism of neuropsychiatric disorders, and generate definitive and quantifiable biological parameters for disease diagnosis and treatment process monitoring. In the future, when sufficient databases have been established linking biological markers with neuropsychiatric disorders, molecular imaging using Positron Emissions Tomography (PET) and labeled AFM and related radioligands might provide a non-invasive and objective means for the diagnosis and evaluation of neuropsychiatric disorders.

The second application of SERT imaging using PET is the study of dose-receptor occupancy relation of therapeutic agents such as the current class of antidepressants known as selective serotonin reuptake inhibitors (SSRIs) that include fluxetine) (Prozac®) and paroxetine (Paxil®). Such an application might also aid in the development of new therapeutic agents by helping define a safe and efficacious therapeutic dose of new agent.

This invention covers the preparation and application of unlabeled and labeled 2-{2-[(dimethylamino)methyl]phenylthio}-5-fluoromethylphenylamine (AFM) and related compounds, which are new chemical entities (NCEs). 2-{2-[(dimethylamino)methyl]phenylthio}-5-fluoromethylphenylamine and related compounds can be labeled with either ¹¹C or ¹⁸F. Combined with Positron Emission Tomography (PET), labeled 2-{2-[(dimethylamino)methyl]phenylthio}-5-fluoromethylphenylamine and related radioligands enables in vivo visualization and quantification of serotonin (5HT) transporters (SERT) in the brain.

The current classes of SERT radioligands use a (¹¹C)radiolabel and ¹¹C has a half-life of 20.4 minutes. Therefore, ¹¹C radiolabeled compounds can only be used in PET Centers with an on-site cyclotron. In the United States, 90% of PET Centers do not have an on-site cyclotron. Our invention allows for [¹⁸F] labeling. The fluorine-18 (¹⁸F) radiolabel has a significantly longer half-life (109.8 minutes) and could be distributed to PET centers through a distribution network that already exists to deliver radioactive compounds across North America. Consequently ¹⁸F labeled imaging compounds would have widespread and feasible application.

This invention provides a compound having the structure:

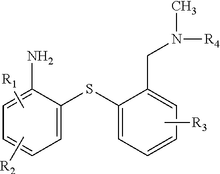

or a physiologically acceptable salt thereof;
wherein R₁ is H, X, CH₃ (CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, (CH₂)ₙCH₃, or (CH₂)ₙCH₂X, where n=0, 1, 2, or 3;
wherein only one of R₁, R₂, and R₃ is H;
wherein X=halogen;
wherein if n=0 then (CH₂)ₙ is absent;
wherein a carbon atom in R₄ may be [¹¹C]; and
wherein any halogen in the compound may be a radioisotope.

In one specific embodiment of the compound one carbon in R₄ is [¹¹C]. In another specific embodiment of the compound one halogen in the compound is a radioisotope. In one specific embodiment of the compound the radioisotope is [¹⁸F]. In another specific embodiment of the compound the halogen in R₁ is [¹⁸F]. In one specific embodiment of the compound wherein R₁ is [¹⁸F]. In another specific embodiment of the compound R₁ is F. In one specific embodiment of the compound R₂ is CH₃.

In one specific embodiment, the compound is

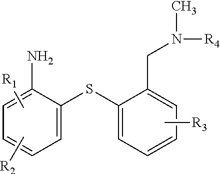

or a physiologically acceptable salt thereof;
R₁ is X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
R₂ is X, (CH₂)ₙCH₃, or (CH₂)ₙCH₂X, where n=0, 1, 2, or 3;
R₃ is H, X, (CH₂)ₙCH₃, or (CH₂)ₙCH₂X, where n=0, 1, 2, or 3;
R₄ is H, or (CH₂)ₙCH₃, where n=0, 1, 2, or 3;
X=halogen;
if n=0 then (CH₂)ₙ is absent;
a carbon atom in R₄ may be [¹¹C]; and
any halogen in the compound may be a radioisotope.

In another specific embodiment the compound is 2-(2-dimethylaminomethylphenylthio)-4-Fluoro-5-methylamine.

In another specific embodiment, the compound is

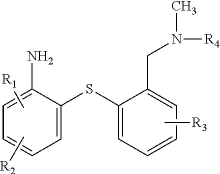

or a physiologically acceptable salt thereof;
R₁ is F, (CH₂)ₙF, ¹⁸F, or (CH₂)ₙ¹⁸F, where n=1, 2, 3, or 4;
R₂ is H, X, (CH₂)ₙCH₃, or (CH₂)ₙCH₂X, where n=0, 1, 2, or 3;
R₃ is H, X, (CH₂)ₙCH₃, or (CH₂)ₙCH₂X, where n=0, 1, 2, or 3;
R₄ is H, or (CH₂)ₙCH₃, where n=0, 1, 2, or 3;
X=halogen;
if n=0 then (CH₂)ₙ is absent;
a carbon atom in R₄ may be [¹¹C]; and
any halogen in the compound may be a radioisotope.

In one specific embodiment, the compound is

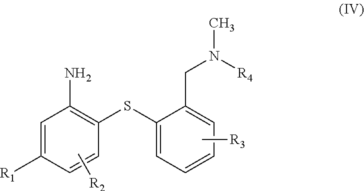

or a physiologically acceptable salt thereof;
R₁ is F, (CH₂)ₙF, ¹⁸F, or (CH₂)ₙ¹⁸F, where n=1, 2, 3, or 4;

### R₂ is H;

### R₃ is H;

R₄ is H, (CH₂)ₙCH₃, (CH₂)ₙCH₂X, (CH₂)ₙ¹¹CH₃, or (CH₂)ₙ¹¹CH₂X where n=0, 1, 2, or 3,
X=halogen; and
n=0 then (CH₂)ₙ is absent.

In one specific embodiment of the compound R₂ is H, F, Br, I, (CH₂)ₙCH₃ or (CH₂)ₙCH₂X. In another specific embodiment of the compound a carbon atom in R₄ is [¹¹C]. In another specific embodiment of the compound R₁ is ¹⁸F and R₄ is CH₃. In one specific embodiment of the compound, R₁ is (i) CH₂¹⁸F, and R₄ is CH₃; (ii) R₁ is (CH₂)₂¹⁸F and R₄ is CH₃; (iii) R₁ is (CH₂)₃¹⁸F and R₄ is CH₃; (iv) R₁ is (CH₂)¹⁸F and R₄ is CH₃; (v) R₁ is ¹⁸F and R₄ is H; (vi) R₁ is (CH₃)¹⁸F and R₄ is H; (vii) R₁ is (CH₂)₂¹⁸F and R₄ is H; (viii) R₁ is (CH₂)₃¹⁸F and R₄ is H; (ix) R₁ is (CH₂)₄¹⁸F and R₄ is H; (x) R₁ is (CH₂ₙ)F where n=0, 1, 2, 3, or 4 and R₄ is ¹¹CH₃; (xi) R₁ is F and R₄ is ¹¹CH₃; (xii) R₁ is CH₂F and R₄ is ¹¹CH₃; (xiii) R₁ is (CH₂)₂F and R₄ is ¹¹CH₃; (xiv) R₁ is (CH₂)₃F and R₄ is (v) R₁ is (CH₂)₄F and R₄ is ¹¹CH₃.

In one specific embodiment the compound is 5-Fluoro-2-((2-((dimethylamino)methyl)phenyl)thio)phenylamine. In another specific embodiment the compound is 2-((2-((methylamino)methyl)phenyl)thio)-5-fluoro-phenylamine. In another specific embodiment the compound is 2-{2-[(dimethylamino)methyl]phenylthio}-5-fluoromethylphenylamine. In another specific embodiment the compound is 2-(2-dimethylaminomethyl-phenylthio)-5-fluoroethylphenylamine. In another specific embodiment the compound is 2-(2-dimethylaminomethyl-phenylthio)-5-fluoropropylphenylamine.

This invention also provides a physiologically acceptable composition comprising the instant compound and a physiologically acceptable carrier.

This invention also provides a process of making a physiologically acceptable composition comprising mixing the compound with a physiologically acceptable carrier.

This invention also provides a process for synthesizing a compound having the structure:

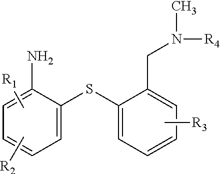

or a physiologically acceptable salt thereof, wherein
R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
R₂ is H, X, (CH₂)ₙCH₃, or (CH₂)ₙCH₂X, where n=0, 1, 2, or 3;
R₃ is H, X, (CH₂)ₙCH₃, or (CH₂)ₙCH₂X, where n=0, 1, 2, or 3;
R₄ is H, or (CH₂)ₙCH₃, where n=0, 1, 2, or 3;
X=halogen;
if n=0 then (CH₂)ₙ is absent;
a carbon atom in R₄ may be [¹¹C]; and
any halogen in the compound may be a radioisotope,
comprising
a) reacting a compound having the structure

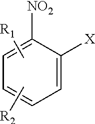

with a compound having the structure

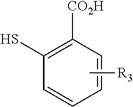

so as to form a product compound having the structure:

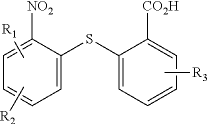

b) reacting the product compound of step a) with CH₃NHR₄ or its salt to form a product compound having the structure:

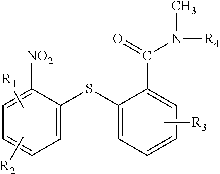

c) reacting the product compound of step b) with a reducing agent to form a product compound having the structure:

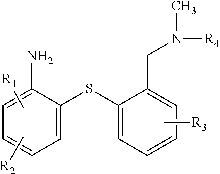

This invention also provides a process for radiolabeling with [¹¹C] a compound having the structure:

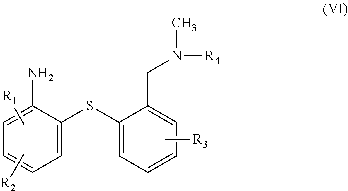

or a physiologically acceptable salt thereof, wherein
R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
R₂ is H, X, (CH₂)ₙCH₃, or (CH₂)ₙCH₂X, where n=0, 1, 2, or 3;
R₃ is H, X, (CH₂)ₙCH₃, or (CH₂)ₙCH₂X, where n=0, 1, 2, or 3;
X=halogen;
if n=0 then (CH₂)ₙ is absent;
a carbon atom in R₄ may be [¹¹C]; and
any halogen in the compound may be a radioisotope,
comprising reacting the compound with [¹¹C]H₃(CH₃I in the presence of a suitable solvent.

This invention also provides a process of radiolabeling with [¹¹C] a compound having the structure:

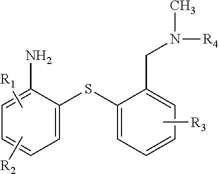

where the suitable solvent is N,N-dimethylformamide (DMF).

This invention also provides a process for radiolabeling with [¹⁸F] a compound having the structure:

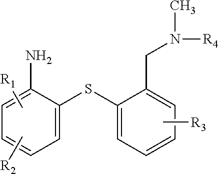

or a physiologically acceptable salt thereof, wherein
R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
R₂ is H, X, (CH₂)ₙCH₃, or (CH₂)ₙCH₂X, where n=0, 1, 2, or 3;
R₃ is H, X, (CH₂)ₙCH₃, or (CH₂)ₙCH₂X, where n=0, 1, 2, or 3;
R₄ is H, (CH₂)ₙCH₃, or (CH₂)ₙCH₂X, where n=0, 1, 2, or 3;
only one of R₁, R₂, and R₃ is H;
X=halogen;
if n=0 then (CH₂)ₙ is absent; and
any halogen in the compound may be a radioisotope,
comprising reacting a compound with a salt of ¹⁸F⁻.

This invention also provides a process for radiolabeling with [¹⁸F] a compound having the structure:

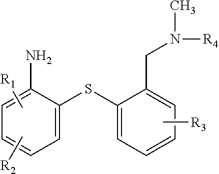

or a physiologically acceptable salt thereof, wherein
R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
R₂ is H, X, (CH₂)ₙCH₃, or (CH₂)ₙCH₂X, where n=0, 1, 2, or 3;
R₃ is H, X, (CH₂)ₙCH₃, or (CH₂)ₙCH₂X, where n=0, 1, 2, or 3;
R₄ is H, (CH₂)ₙCH₃, or (CH₂)ₙCH₂X, where n=0, 1, 2, or 3;
either one or two of R₁, R₂, and R₃ is H;
X=halogen;
if n=0 then (CH₂)ₙ is absent; and
any halogen in the compound may be a radioisotope,
comprising reacting a compound with K¹⁸F.

This invention also provides a non-invasive method for positron emission tomography (PET) imaging of serotonin transporter sites in a mammal comprising labeling serotonin transporter sites (SERT) with an image-generating amount of the instant radiolabeled compound and measuring spatial distribution of the compound in the mammal by PET so as to thereby image the serotonin transporter sites.

The invention is illustrated but not limited by the following examples.

### EXPERIMENTAL DETAILS

Given the limitations of [¹¹C]McN 5652 and other radioligands discussed in the Background of the Invention, the most decisive improvement criteria for a new radioligand include a faster kinetic of uptake and higher target to background ratio. Faster uptake kinetics will enable time-independent derivation of SERT parameters in shorter scanning sessions and a higher target to background ratio would improve the reliability in the quantification of SERT availability in regions of relatively low SERT density, but of great significance in psychiatric conditions, such as the limbic system. In addition, a new radioligand that can be labeled with the longer-lived radioisotope fluorine-18 is also advantageous in that it enables the wide distribution and application of the ligand.

### I. Preparation of Compounds

### A. Synthesis

Preparation of compounds of structure (I) utilizes the established art of organic chemistry and is illustrated but not limited by the following schematic representation (Scheme 1):

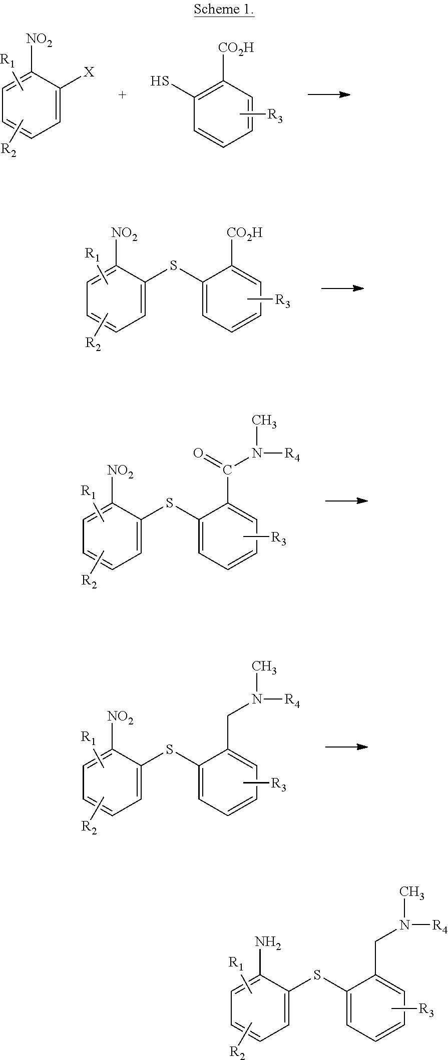

### B. Examples

Herein, the terms “intermediate” and “example”, when used as headers and in reference to compounds, are equivalent.

### Intermediate 1: 2-(4-Methyl-2-nitrophenylthio)benzoic acid

A mixture of 4-bromo-3-nitrotoluene (4.54 g, 21 mmol), thiosalicylic acid (3.1 g, 20 mmol), Cu powder (365 mg) and K₂CO₃ (6.4 g) in DMF (50 mL) was heated at 65° C. overnight, cooled to room temperature and poured to ice water. The mixture was filtered through a layer of Celite. The filtrate was made acidic with the addition of 6 N HCl and extracted with CH₂Cl₂ (70 mL×3). The combined organic layers were washed once with H₂O, dried, and concentrated. The crude product was crystallized from EtOH/H₂O to provide the title compound as a yellowish solid (4.97 g, 86%), mp 189-191° C. ¹H NMR: δ 8.13 (dd, 1H, J=1.6, 7.7 Hz), 7.93 (s, 1H), 7.51 (1H, J=1.6, 7.6 Hz), 7.44 (dt, 1H, J=1.2, 7.6 Hz), 7.25-7.35 (m, 2H), 7.13 (d, J=8.1 Hz), 2.47 (s, 3H). Anal. Calcd for C₁₄H₁₁NO₄S: C, 58.12; H, 3.83; N, 4.84. Found: C, 57.74; H, 3.78; N, 4.59.

### Intermediate 2: 2-(4-Fluoro-2-nitrophenylthio)benzoic acid

In an analogous manner the title compound was prepared from 2-chloro-5-fluoronitrobenzene and thiosalicylic acid in 40% yield as a yellowish solid, mp 167-168° C. ¹H NMR: δ 8.13 (dd, 1H, J=1.2, 7.7 Hz), 7.86 (dd, 1H, J=2.3, 8.0 Hz), 7.45-7.58 (m, 2H), 7.30-7.38 (m, 1H), 7.18-7.25 (m, 2H). Anal. Calcd for C₁₃H₈FNO₄S: C, 53.24; H, 2.75; N, 4.78. Found: C, 53.09; H, 2.77; N, 4.43.

### Intermediate 3: 2-(2,4-Dinitrophenylthio)benzoic acid

In an analogous manner the title compound was prepared from bromo-2,4-dinitrobenzene and thiosalicylic acid in 20% yield as a yellow solid, mp 179-181° C. ¹H NMR: δ 9.07 (d, 1H, J=2.4 Hz), 8.18-8.08 (m, 2H), 7.76-7.64 (m, 3H), 6.97 (d, 1H, J=9.0 Hz).

### Intermediate 4: 2-(5-Fluoro-4-methyl-2-nitrophenylthio)benzoic acid

In an analogous manner the title compound was prepared from 4-chloro-2-fluoro-5-nitrotoluene and thiosalicylic acid in 60.6% as a yellowish solid. ¹H NMR: δ 8.09 (m, 2H), 7.55-7.57 (m, 3H), 6.61 (d, 1H, J=7.6 Hz), 2.31 (s, 3H).

### Intermediate 5: 2-(4-Acetyloxymethyl-2-nitrophenylthio)benzoic acid

In an analogous manner the title compound was prepared from 4-chloro-3-nitrobenzyl acetate and thiosalicylic acid in 39% yield as a yellowish solid, mp 150-151° C. ¹H NMR: δ 8.16 (d, 1H, J=1.1 Hz), 8.12 (dd, 1H, J=1.1, 7.2 Hz), 7.60-7.38 (m, 4H), 7.08 (d, 1H, J=8.3 Hz), 5.15 (s, 2H), 2.15 (s, 3H). Anal. Calcd for C₁₆H₁₃NO₆S: C, 55.33; H, 3.77; N, 4.03. Found: C, 55.21; H, 3.80; N, 3.91.

### Intermediate 6: 2-(4-Methoxycarbonylmethyl-2-nitrophenyl-thio)benzoic acid

In an analogous manner the title compound was prepared from methyl 4-bromo-3-nitrophenylacetate and thiosalicylic acid in 56% yield as a yellowish solid, mp 135-136° C. ¹H NMR: δ 3.67 (s, 2H), 3.72 (s, 3H), 7.06 (d, 1H, J=8.3 Hz), 7.34-7.54 (m, 4H), 8.06 (m, 2H). Anal. Calcd for C₁₆H₁₃NO₆S: C, 55.33; H, 3.77; N, 4.03. Found: C, 55.30; H, 3.76; N, 4.06.

### Intermediate 7: 2-(4-Methoxycarbonylethyl-2-nitrophenyl-thio)benzoic acid

In an analogous manner the title compound was prepared from methyl 3-(4-chloro-3-nitrophenyl)propionate and thiosalicylic acid in 46% yield as a yellowish solid, mp 160-161° C. ¹H NMR: δ 2.61 (t, 2H, J=7.6), 2.85 (s, 3H), 2.94 (t, 2H, J=7.6), 3.03 (s, 3H), 3.66 (s, 3H), 6.87 (d, 1H, J=8.4 Hz), 7.22 (dd, 1H, J=1.77, 8.4 Hz), 7.40-7.58 (m, 4H), 7.85 (d, 1H, J=1.6 Hz), 8.09 (dd, 1H, J=1.5, 7.7 Hz). Anal. Calcd for C₁₇H₁₅NO₆S: C, 56.50; H, 4.18; N, 3.88. Found: C, 56.37; H, 4.10; N, 3.82.

### Intermediate 8: 2-(4-Methyl-2-nitrophenylthio)-N,N-dimethylbenzamide

A solution of intermediate 1 (2.0 g, 6.9 mmol) in thionyl chloride (20 mL) was heated at 70° C. for 3 hours, cooled to room temperature and the excess thionyl chloride removed in vacuo. The residue was redissolved in THF (25 mL). And to this solution was added N,N-dimethylamine hydrochloride (1.2 g, 13.8 mmol) and K₂CO₃ (1.9 g, 13.8 mmol). The reaction mixture was stirred overnight at room temperature, diluted with H₂O and extracted with CH₂Cl₂ (60 mL×4). The combined organic layers were washed once with H₂O, dried and concentrated. Column chromatography of the crude products on silica gel and elution with EtOAc/hexane (60:40) afforded the title compound (2.04 mg, 93%) as a yellow solid, mp 105-106° C. ¹H NMR: δ 8.02 (d, 1H, J=1.5 Hz), 7.40-7.63 (m, 4H), 7.21 (dd, 1H, J=1.5, 8.3 Hz), 6.88 (d, 1H, J=8.3 Hz), 3.08 (s, 3H), 2.88 (s, 3H), 2.36 (s, 3H). Anal. Calcd for C₁₆H₁₆N₂O₃S: C, 60.74; H, 5.10; N, 8.85. Found: C, 60.98; H, 5.07; N, 8.99.

### Intermediate 9: 2-(4-Methyl-2-nitrophenylthio)-N-methylbenzamide

In an analogous manner the title compound was prepared from intermediate 1 and N-methylamine hydrochloride in 71% yield as a yellow solid, mp 122-123° C. ¹H NMR: δ 8.02 (d, 1H, J=1.0 Hz), 7.82 (dd, 1H, J=1.6, 7.6 Hz), 7.62 (dd, 1H, J=1.3, 7.6 Hz), 7.57 (dt, 1H, J=1.3, 7.6 Hz), 7.51 (dt, 1H, J=1.6, 7.6 Hz), 7.23 (dd, 1H, J=1.0, 8.3 Hz), 6.83 (d, 1H, J=8.3 Hz), 6.42 (br s, 1H), 2.87 (d, 3H, J=4.9 Hz), 2.40 (s, 3H). Anal. Calcd for C₁₅H₁₆N₂O₃S: C, 59.59; H, 4.67; N, 9.27. Found: C, 59.34; H, 4.59; N, 9.18.

### Intermediate 10: 2-(4-Fluoro-2-nitrophenylthio)-N,N-dimethylbenzamide

In an analogous manner the title compound was prepared from intermediate 2 and N,N-dimethylamine hydrochloride in 93% yield as a yellow solid, mp 56-57° C. ¹H NMR: δ 7.90 (dd, 1H, J=2.8, 8.3 Hz), 7.55-7.65 (m, 2H), 7.49 (dt, 1H, J=1.5, 7.6 Hz), 7.43 (dd, 1H, J=1.2, 7.6 Hz), 7.15 (ddd, 1H, J=2.8, 7.2, 9.1 Hz), 6.96 (dd, 1H, J=5.2, 9.1 Hz), 3.08 (s, 3H), 2.88 (s, 3H). Anal. Calcd for C₁₅H₁₃FN₂O₃S: C, 56.24; H, 4.09; N, 8.74. Found: C, 56.46; H, 4.08; N, 8.58.

### Intermediate 11: 2-(4-Fluoro-2-nitrophenylthio)-N-methylbenzamide

In an analogous manner the title compound was prepared from intermediate 2 and N-methylamine hydrochloride in 97% as a yellow solid, mp 111-113° C. ¹H NMR: δ 7.94 (dd, 1H, J=2.8, 8.2 Hz), 7.75 (dd, 1H, J=1.6, 7.6 Hz), 7.50-7.63 (m, 3H), 7.17 (dd, 1H, J=2.8, 7.2, 9.1 Hz), 6.96 (dd, 1H, J=5.2, 9.0 Hz), 6.20 (br, 1H), 2.90 (d, 3H, J=4.9 Hz). Anal. Calcd for C₁₄H₁₁FN₂O₃S: C, 54.89; H, 3.62; N, 9.15. Found: C, 55.00; H, 3.65; N, 8.90.

### Intermediate 12: 2-(2,4-Dinitrophenylthio)-N,N-dimethylbenzamide

In an analogous manner the title compound was prepared from intermediate 3 and N,N-dimethylamine hydrochloride in 85% yield as a yellow solid, mp 144-145° C. ¹H NMR: δ 9.06 (d, 1H, J=2.4 Hz), 8.18-8.10 (dd, 1H, J=2.4, 9.1 Hz), 7.66-7.44 (m, 4H), 7.06 (d, 1H, J=9.1 Hz), 3.03 (s, 3H), 2.87 (s, 3H).

### Intermediate 13: 2-(5-Fluoro-4-methyl-2-nitrophenylthio)-N,N-dimethylbenzamide

In an analogous manner the title compound was prepared from intermediate 4 and N,N-dimethylamine hydrochloride in 85% yield as a yellow solid. ¹H NMR: δ 2.27 (s, 3H), 2.87 (s, 3H), 3.07 (s, 3H), 6.53 (d, 1H, J=10.32 Hz), 7.45-7.63 (m, 4H), 8.13 (d, 1H, J=7.06 Hz).

### Intermediate 14: 2-(4-Acetyloxymethyl-2-nitrophenylthio)-N,N-dimethylbenzamide

In an analogous manner the title compound was prepared from intermediate 5 and N,N-dimethylamine hydrochloride in 58% yield as a yellow solid, mp 117-118° C. ¹H NMR: δ 8.22 (d, 1H, J=0.9 Hz), 7.63-7.42 (m, 4H), 7.37 (dd, 1H, J=1.5, 8.4 Hz), 6.95 (d, 1H, J=8.4 Hz), 5.09 (s, 2H), 3.07 (s, 3H), 2.88 (s, 3H), 2.12 (s, 3H). Anal. Calcd for C₁₈H₁₈N₂O₅S: C, 57.74; H, 4.85; N, 7.48. Found: C, 57.96; H, 4.82; N, 7.39.

### Intermediate 15: 2-(4-Acetyloxymethyl-2-nitrophenylthio)-N-methylbenzamide

In an analogous manner the title compound was prepared from intermediate 5 and N-methylamine hydrochloride in 84% yield as a yellowish solid, mp 111-113° C. ¹H NMR: δ 8.21 (d, 1H, J=1.7 Hz), 7.75 (dd, 1H, J=1.7, 7.5 Hz), 7.68-7.48 (m, 3H), 7.36 (dd, 1H, J=1.8, 8.4 Hz), 6.90 (d, 1H, J=8.4 Hz), 6.32 (br s, 1H), 5.09 (s, 2H), 2.87 (d, 3H, J=4.8 Hz). Anal. Calcd for C₁₇H₁₆N₂O₅S: C, 56.66; H, 4.47. N, 7.77. Found: C, 56.58; H, 4.45; N, 7.71.

### Intermediate 16: 2-(4-Methoxycarbonylmethyl-2-nitrophenyl-thio)-N,N-dimethylbenzamide

In an analogous manner the title compound was prepared from intermediate 6 and N,N-dimethylamine hydrochloride in 53% yield as a yellowish solid, mp 124-125° C. ¹H NMR: δ 2.85 (s, 3H), 3.05 (s, 3H), 3.62 (s, 2H), 3.69 (s, 3H), 6.89 (d, 1H, J=8.4 Hz), 7.26-7.59 (m, 5H), 8.12 (d, 1H, J=1.9 Hz). Anal. Calcd for C₁₈H₁₈N₂O₅S: C, 57.74; H, 4.85; N, 7.48. Found: C, 57.56; H, 4.78; N, 7.43.

### Intermediate 17: 2-(4-Methoxycarbonylmethyl-2-nitrophenyl-thio)-N-methylbenzamide

In an analogous manner the title compound was prepared from intermediate 6 and N-methylamine hydrochloride in 63% yield as a yellowish solid. ¹H NMR: δ 2.87 (s, 3H), 3.64 (s, 2H), 3.70 (s, 3H), 6.33 (s, 1H), 6.86 (d, 1H, J=8.38 Hz), 7.24-7.78 (m, 5H), 8.13 (d, 1H, J=1.58 Hz).

### Intermediate 18: 2-(4-(2-(Methoxycarbonylethyl))-2-nitro-phenylthio)-N,N-dimethylbenzamide

In an analogous manner the title compound was prepared from intermediate 7 and N,N-dimethylamine hydrochloride in 60% yield as a yellow oil. ¹H NMR: δ 2.57 (t, 2H, J=7.57 Hz), 3.02 (t, 2H, J=7.54 Hz), 3.69 (s, 3H), 7.07 (d, 1H, J=8.2 Hz), 7.28-7.52 (m, 4H), 7.85 (d, 1H, J=1.6 Hz), 8.03 (d, 1H, J=1.6 Hz).

### Intermediate 19: (2-(4-Methyl-2-nitrophenylthio)benzyl)dimethylamine

To a solution of intermediate 8 (1.3 g, 4.2 mmol) in THF (10 mL), cooled at 0° C., was introduced the BH₃.THF complex (45 mL, 1M solution in THF, 10.0 mmol) via a syringe. The reaction mixture was refluxed at 70° C. for 2 hours and then stirred overnight at room temperature. The reaction mixture was cooled to 0° C. and concentrated HCl added. The solvent was removed in vacuo. And the aqueous phase was diluted with H₂O (20 mL), heated to reflux for 20 minutes, and, after cooling down to room temperature, adjusted to pH 7-8 with 10% NaHCO₃. The mixture was extracted with CH₂Cl₂ (30 mL×4). The combined organic layers were dried and concentrated in vacuo. Column chromatography on silica gel and elution with EtOAc/hexane (60:40) afforded the title compound (1.05 g, 83%) as a yellow solid, mp 109-111° C. ¹H NMR: δ 8.09 (d, 1H, J=1.5 Hz), 7.78 (dd, 1H, J=1.5, 7.6 Hz), 7.65 (dd, 1H, J=1.5, 7.6 Hz), 7.58 (dt, 1H, J=1.5, 7.6 Hz), 7.51 (dt, 1H, J=1.5, 7.6 Hz), 7.17 (dd, 1H, J=1.5, 8.3 Hz), 6.44 (d, 1H, J=8.3 Hz), 4.24 (s, 2H), 2.65 (s, 6H), 2.40 (s, 3H). Anal. Calcd for C₁₆H₁₈N₂O₂S: C, 63.55; H, 6.00; N, 9.26. Found: C, 63.24; H, 6.00; N, 8.98.

### Intermediate 20: (2-(4-Methyl-2-nitrophenylthio)benzyl)methyl amine

In an analogous manner the title compound was prepared from intermediate 9 in 91% yield as a yellow thick oil. ¹H NMR: δ 8.11 (m, 1H), 7.50-7.65 (m, 3H), 7.39 (m, 1H), 7.18 (m, 1H), 6.60 (m, 1H), 3.86 (s, 2H), 2.42 (s, 3H), 2.38 (s, 3H). Anal. Calcd for C₁₅H₁₆N₂O₂S: C, 62.48; H, 5.59; N, 9.71. Found: C, 62.16; H, 5.61; N, 9.52

### Intermediate 21: (2-(4-Fluoro-2-nitrophenylthio)benzyl)dimethylamine

In an analogous manner the title compound was prepared from intermediate 10 in 55% yield as a yellowish solid. ¹H NMR: δ 7.98 (dd, 1H, J=2.8, 8.4 Hz), 7.67 (d, 1H, J=7.7 Hz), 7.56 (d, 1H, J=7.6 Hz), 7.52 (t, 1H, J=7.6 Hz), 7.47 (t, 1H, J=7.6 Hz), 7.1 (ddd, 1H, J=2.8, 7.2, 9.1 Hz), 6.72 (dd, 1H, J=5.2, 9.1 Hz), 3.55 (s, 2H), 2.20 (s, 3H). Anal. Calcd for C₁₅H₁₅FN₂O₈S: C, 58.81; H, 4.94; N, 9.14. Found: 58.60; H, 4.99; N, 8.89.

### Intermediate 22: (2-(4-Fluoro-2-nitrophenylthio)benzyl)methylamine

In an analogous manner the title compound was prepared from intermediate 11 in 85% yield as a yellow thick oil. ¹H NMR: δ 8.20 (dd, 1H, J=2.8, 8.3 Hz), 7.63 (dd, 1H, J=1.2, 7.6 Hz), 7.58 (dd, 1H, J=1.2, 7.6 Hz), 7.44 (dt, 1H, J=1.2, 7.6 Hz), 7.40 (dt, 1H, J=1.2, 7.6 Hz), 7.12 (ddd, 1H, J=2.8, 7.2, 9.1 Hz), 6.71 (dd, 1H, J=5.1, 9.1 Hz), 3.87 (s, 2H), 2.43 (s, 3H). An analytical sample was prepared by reacting the title compound with equal mole of (dl)-tartaric acid in acetone to give the tartrate salt of title compound as colorless solid, mp 171-172° C. Anal. Calcd for C₁₄H₁₃FN₂O₂S.C₄H₆O₆: C, 48.87; H, 4.33; N, 6.33. Found: C, 48.79; H, 4.30; N, 6.16.

### Intermediate 23: (2-(2,4-Dinitrophenylthio)-N,N-dimethylbenzylamine

In an analogous manner the title compound was prepared from intermediate 12 in 52% yield as a yellowish solid, mp 128-129° C. ¹H NMR: δ 9.11 (d, 1H, J=2.5 Hz), 8.08 (dd, 1H, J=2.5, 9.1 Hz), 7.69-7.38 (m, 4H), 6.82 (d, 1H, J=9.1 Hz), 3.50 (s, 2H), 2.15 (s, 6H).

### Intermediate 24: (2-(5-Fluoro-4-methyl-2-nitrophenylthio)-N,N-dimethylbenzylamine

In an analogous manner the title compound was prepared from intermediate 13 in 77.8% yield as a yellowish solid. ¹H NMR: δ 2.22 (s, 6H), 2.28 (s, 3H), 3.55 (s, 2H), 6.28 (d, 1H, J=10.55 Hz), 7.36-7.70 (m, 4H), 8.19 (d, 1H, J=7.14 Hz).

### Intermediate 25: 2-(4-Hydroxymethyl-2-nitrophenylthio)-N,N-dimethylbenzylamine

In an analogous manner the title compound was prepared from intermediate 14 in 67% yield as a yellowish thick oil. ¹H NMR: δ 8.22 (d, 1H, J=1.1 Hz), 7.64 (d, 1H, J=8.1 Hz), 7.58-7.43 (m, 2H), 7.38-7.25 (m, 2H), 6.67 (d, 1H, J=8.4 Hz), 4.69 (s, 2H), 3.54 (s, 2H), 2.19 (s, 6H). HRMS: calcd for C₁₆H₁₉N₂O₃S m/z (MH⁺): 319.1116; found: 319.1104. Anal. Calcd C₁₆H₁₉N₂O₃S.0.2H₂O: C, 59.94; H, 5.78; N, 8.63. Found: C, 59.82; H, 5.72; N, 8.52.

### Intermediate 26: 2-(4-Hydroxymethyl-2-nitrophenylthio)-N-methylbenzylamine

In an analogous manner the title compound was prepared from intermediate 15 in 79% yield as a yellowish thick oil. ¹H NMR: δ 8.25 (d, 1H, J=0.98 Hz), 7.62-7.48 (m, 3H), 7.42-7.28 (m, 2H), 6.68 (d, 1H, J=8.4 Hz), 4.70 (s, 2H), 3.81 (s, 2H), 2.39 (s, 3H). HRMS: calcd for C₁₅H₁₇N₂O₃S m/z (MH): 305.0960; found: 305.0930. Anal. Calcd for C₁₅H₁₇N₂O₃S.0.5H₂O: C, 57.49; H, 5.47; N, 8.94. Found: C, 57.17; H, 5.09; N, 8.64.

### Intermediate 27: 2-(4-(2-(Hydroxyethyl)-2-nitrophenylthio)-N,N-dimethylbenzylamine

In an analogous manner the title compound was prepared from intermediate 16 in 80% yield as a yellowish thick oil. ¹H NMR: δ 2.20 (s, 6H), 2.87 (t, 2H, J=6.39 Hz), 3.55 (s, 2H), 3.87 (t, 2H, J=6.40 Hz), 7.85 (d, 1H, J=1.6 Hz), 6.62 (d, 1H, J=8.33 Hz), 7.18-7.66 (m, 5H), 8.12 (d, 1H, J=1.74 Hz). Anal. Calcd for C₁₇H₂₀N₂O₃S.0.33H₂O: C, 60.33; H, 6.16; N, 8.28. Found: C, 60.68; H, 6.07; N, 8.28.

### Intermediate 28: 2-(4-(2-(Hydroxyethyl)-2-nitrophenylthio)-N-methylbenzylamine

In an analogous manner the title compound was prepared from intermediate 17 in 62% yield as a yellowish oil. ¹H NMR: δ 1.71 (s, 2H), 2.39 (s, 3H), 2.86 (t, 3H, J=6.4), 3.82-3.87 (m, 4H), 6.63 (d, 1H, J=8.3 Hz), 7.29-7.57 (m, 5H), 8.14 (d, 1H, J=1.8 Hz).

### Intermediate 29: 2-(4-(3-Hydroxypropyl)-2-nitrophenylthio)-N,N-dimethylbenzylamine

In an analogous manner the title compound was prepared from intermediate 18 in 60% yield as a yellowish thick oil. ¹H NMR: δ 1.85-1.89 (m, 2H), 2.20 (s, 3H), 2.73 (t, 2H, J=7.9 Hz), 3.54 (s, 3H), 3.66 (t, 2H, J=6.3 Hz), 6.62 (d, 1H, J=8.4 Hz), 7.15 (dd, 1H, J=1.6, 8.4 Hz) 7.33 (td, 1H, J=1.1, 7.5 Hz), 7.85 (d, 1H, J=1.6 Hz), 7.48 (td, 1H, J=1.1, 7.6 Hz), 7.53 (dd, 1H, J=0.9, 7.7 Hz), 7.66 (d, 1H, J=7.7 Hz), 8.07 (d, 1H, J=1.7 Hz). Anal. Calcd for C₁₈H₂₂N₂O₃S: C, 62.40; H, 6.40; N, 8.09. Found: C, 62.20; H, 6.51; N, 8.03.

### Intermediate 30: 2-(4-Fluoromethyl-2-nitrophenylthio)-N,N-dimethylbenzylamine

To a solution of intermediate 25 (228 mg, 0.72 mmol) in CH₂Cl₂ (10 mL) was added [bis(2-methoxyethyl)amino]sulfur trifluoride (0.15 mL, 0.80 mmol) at 0° C. The reaction was stirred for 2 hours at room temperature. The reaction mixture was then diluted with CH₂Cl₂ (30 mL) and washed with 10% NaHCO₃ (25 mL×3). The NaHCO₃ washes were combined and back washed with CH₂Cl₂ once. The organic layers were washed with H₂O once, dried and concentrated. The crude product was purified by column chromatography on silica gel. Elution with MeOH/CH₂Cl₂. (5:95) gave the title compound as a yellow thick oil (76 mg, 33%). ¹H NMR: δ 8.25 (s, 1H), 7.65 (d, 1H, J=7.7 Hz), 7.55 (d, 1H, J=7.7 Hz), 7.50 (dt, 1H, J=1.3, 7.6 Hz), 7.35 (dt, 1H, J=1.3, 7.6 Hz), 7.30 (d, 1H, J=8.4 Hz), 6.72 (d, 1H, J=8.4 Hz), 5.36 (d, 2H, J=47.3 Hz), 3.53 (s, 2H), 2.19 (s, 6H). Anal. Calcd for C₁₆H₁₇FN₂O₂S: C, 59.98; H, 5.35; N, 8.74. Found: C, 60.02; H, 5.35; N, 8.43.

### Intermediate 31: 2-(4-fluoromethyl-2-nitrophenylthio)-N-methylbenzylamine

In an analogous manner the title compound was obtained from intermediate 26 in 44% yield as a yellowish oil. ¹H NMR: δ 8.29 (s, 1H), 7.65 (d, 1H, J=7.6 Hz), 7.60 (t, 1H, J=7.6 Hz), 7.40 (t, 1H, J=7.6 Hz), 7.34 (d, 1H, J=8.4 Hz), 6.75 (d, 1H, J=8.4 Hz), 5.38 (d, 2H, J=47.3 Hz), 3.86 (s, 2H), 2.42 (s, 3H). HRMS: calcd for C₁₅H₁₆FN₂O₂S m/z (MH⁺): 307.0917. Found: 307.0941. Anal. Calcd for C₁₅H₁₆FN₂O₂S: C, 58.81; H, 4.94; N, 9.14. Found: C, 58.57; H, 5.03; N, 8.97.

### Intermediate 32: 2-(4-(2-fluoroethyl)-2-nitrophenylthio)-N,N-dimethylbenzylamine

In an analogous manner the title compound from was obtained intermediate 27 in 52% yield as a yellowish oil. ¹H NMR: δ 1.93-2.06 (m, 2H), 2.2 (s, 6H), 3.00 (dt, 2H, J=6.0, 25.7 Hz), 3.55 (s, 2H), 4.63 (dt, 2H, J=6.0, 46.9 Hz), 6.63 (d, 1H, J=8.4 Hz), 7.18-7.66 (m, 5H), 8.12 (d, 1H, J=1.7 Hz). Anal. Calcd for C₁₇H₁₉FN₂O₂S: C, 61.06; H, 5.73; N, 8.38. Found: C, 60.76; H, 5.74; N, 8.10.

### Intermediate 33: 2-(4-(2-fluoroethyl)-2-nitrophenylthio)-N-methylbenzyl amine

In an analogous manner the title compound from was obtained intermediate 28 in 65% yield as a yellowish oil. ¹H NMR: δ 2.52 (d, 3H, J=3.4 Hz), 2.92 (dt, 2H, J=6.5, 23.7 Hz), 3.95 (s, 2H), 4.59-4.71 (dt, 2H, J=6.5, 47.1 Hz), 6.64 (d, 1H, J=9.4 Hz), 6.66 (s, 1H), 6.83 (m, 1H), 7.10 (m, 2H), 7.31-7.35 (m, 2H).

### Intermediate 34: 2-(4-(3-fluoropropyl)-2-nitrophenylthio)-N,N-dimethylbenzylamine

In an analogous manner the title compound was obtained from intermediate 29 in 88% yield as a yellowish oil ¹H NMR: δ 1.93-2.06 (m, 2H), 2.31 (s, 6H), 2.68 (t, 2H, J=7.8 Hz), 3.56 (s, 2H), 4.4 (dt, 2H, J=5.8, 47.1 Hz), 6.63 (d, 1H, J=8.4 Hz), 7.16 (dd, 1H, J=1.8, 8.4 Hz) 7.49 (t, 1H, J=7.6 Hz), 7.53 (d, 1H, J=7.6 Hz), 7.64 (d, 1H, J=7.5 Hz), 8.08 (d, 1H, J=1.6 Hz). Anal. Calcd for C₁₈H₂₁FN₂O₂S.0.25 H₂O: C, 61.41; H, 6.13; N, 7.96. Found: C, 61.69; H, 6.08; N, 7.90.

### Intermediate 35: 2-(4-Chloromethyl-2-nitrophenylthio)-N,N-dimethylbenzylamine

To a solution of intermediate 25 (10 mg, 0.031 mmol) in anhydrous chloroform (5 mL) was added triethylamine (5 μL, 0.031 mmol), and thionyl chloride (300 μL). The reaction mixture was heated at reflux for 2 hours, cooled to room temperature and washed with ice water. The aqueous phase was removed. The organic layer was neutralized with 10% aqueous NaHCO₃ and extracted with CH₂Cl₂ (3×5 mL). The combined organic layers were washed once with H₂O, dried, and concentrated to give the title compound (7.0 mg, 66%) as a yellowish thick oil. ¹H NMR: δ 8.28 (d, 1H, J=2 Hz), 7.80-7.30 (m, 5H), 6.68 (d, 1H, J=8.4 Hz), 4.56 (s, 2H), 3.20 (s, 2H), 2.19 (s, 6H).

### Intermediate 36: 4-Toluene sulfonic acid 2-[3-nitro-4-(2-dimethylaminomethylphenylthio)phenyl]ethyl ester

To a solution of intermediate 27 (17 mg, 0.051 mmol) in CH₂Cl₂ (2 mL) was added toluenesulfonyl chloride (11 mg, 0.056 mmol), and pyridine (5.4 μl, 0.067 mmol). The reaction mixture was stirred at room temperature for 2 hours and washed with 10% of aqueous Na₂CO₃. The organic layer was dried and concentrated. The crude product was purified by silica gel column with MeOH/CH₂Cl₂ (2:98) to provide the title compound (13 mg, 60.4%) as a yellowish oil. ¹H NMR: δ 2.19 (s, 6H), 2.91 (t, 2H, J=6.6 Hz), 3.52 (s, 2H), 4.17 (t, 2H, J=6.5 Hz), 4.23 (s, 2H), 4.30 (s, 2H), 6.59 (d, 1H, J=8.4 Hz), 7.04-7.66 (m, 10H), 7.99 (d, 1H, J=1.7 Hz).

### Intermediate 37: 4-Toluene sulfonic acid 3-[3-nitro-4-(2-dimethylaminomethylphenylthio)phenyl]propyl ester

In an analogous manner the title compound was prepared from intermediate 29 in 58.8% yield as a yellowish oil. ¹H NMR: δ 1.91-1.97 (m, 8H), 2.63-2.66 (m, 8H), 4.04 (t, 2H, J=6.1 Hz), 4.23 (s, 2H), 4.39 (s, 2H), 6.42 (d, 1H, J=8.4 Hz), 7.08-7.78 (m, 10H), 8.03 (d, 1H, J=1.9 Hz).

### Example 38

### 2-(2-(Dimethylaminomethyl)phenylthio)-5-methylphenylamine

To a solution of intermediate 19 (100.0 mg, 0.33 mmol) in MeOH (4 mL) was added concentrated HCl (2 mL). The suspension was cooled to 0° C., SnCl₂ (378 mg, 1.98 mmol) was added and the reaction mixture stirred overnight at room temperature under nitrogen. The mixture was then diluted with H₂O (10 mL), and extracted with EtOAc (10 mL×2). The organic layers were discarded. The aqueous layer was made basic to pH 10 with 1N NaOH and extracted with EtOAc (15 mL×4). The combined organic layers were washed once with H₂O, dried, and concentrated in vacuo to give the title compound (66.2 mg, 73%) as a colorless oil. ¹H NMR: δ 7.43 (d, 1H, J=7.7 Hz), 7.22-7.32 (m, 1H), 7.05-7.15 (m, 2H), 6.88 (m, 1H), 6.55-6.65 (m, 2H), 4.42 (br s, 2H), 3.62 (s, 2H), 2.38 (s, 6H), 2.32 (3, 3H). An analytical sample was prepared by reaction of the title compound with equal moles of (dl)-tartaric acid in acetone to give the tartrate salt as a colorless solid, mp 159-161° C. HRMS: Calcd for C₁₆H₂₁N₂S (MH+): m/z 273.1425; Found: 273.1409.

### Example 39

### 5-Methyl-2-(2-(methylaminomethyl)phenylthio)phenylamine

In an analogous manner the title compound was prepared from intermediate 20 in 62% yield as a colorless oil. ¹H NMR: δ 7.26-7.38 (m, 2H), 7.04-7.18 (m, 2H), 6.78-6.92 (m, 1H), 6.57-6.70 (m, 2H), 4.25 (br, 2H), 3.95 (s, 2H), 2.53 (s, 3H), 2.32 (s, 3H). An analytical sample was prepared by reacting the title compound with equal moles of (dl)-tartaric acid in acetone to give the tartrate salt as a colorless solid, mp 164-166° C. HRMS: Calcd for C₁₅H₁₉N₂S (MH⁺): m/z 259.1269; Found: 259.1265.

### Example 40

### 2-(2-(Dimethylaminomethyl)phenylthio)-5-fluorophenylamine

In an analogous manner the title compound was prepared from intermediate 21 in 74% yield as colorless oil. ¹H NMR: δ 7.48 (m, 1H), 7.23 (m, 1H), 7.07-7.18 (m, 2H), 6.90 (m, 1H), 6.40-6.53 (m, 2H), 4.80 (br s, 2H), 3.60 (s, 2H), 2.32 (s, 6H). An analytical sample was prepared by reacting the title compound with equal moles of (dl)-tartaric acid in acetone to give the tartrate salt as a colorless solid, mp 158-159° C. Anal. Calcd for C₁₅H₁₇FN₂S.C₄H₆O₆: C, 53.51; H, 5.44; N, 6.57. Found: C, 53.37; H, 5.52; N, 6.49.

### Example 41

### 5-Fluoro-2-(2-(methylaminomethyl)phenylthio)phenylamine

In an analogous manner the title compound was prepared from intermediate 22 in 53% yield as colorless oil. ¹H NMR: δ 7.44 (dd, 1H, J=6.3, 9.1 Hz), 7.33 (m, 1H), 7.06-7.18 (m, 2H), 6.83 (m, 1H), 6.44-6.54 (m, 2H), 4.60 (br s, 2H), 3.95 (s, 2H), 2.55 (s, 3H). An analytical sample was prepared by reacting the title compound with equal moles of (dl)-tartaric acid in acetone to give the tartrate salt as a colorless solid, mp 106-108° C. HRMS: Calcd for C₁₄H₁₆FN₂S (MH⁺): m/z 263.1018; Found: 263.1035. Anal. Calcd for C₁₄H₁₅FN₂S.0.33H₂O: C, 62.66; H, 5.88; N, 10.44. Found: C, 62.81; H, 5.62; N, 10.39.

### Example 42

### 2-(2-(Dimethylaminomethyl)phenylthio)-4-fluoro-5-methylphenylamine

In an analogous manner the title compound was prepared from intermediate 24 in 87.9% yield as colorless oil. ¹H NMR: δ 2.28 (s, 3H), 2.34 (s, 6H), 3.75 (s, 2H), 6.21 (d, 1H, J=10.4 Hz), 7.43-7.60 (m, 3H), 7.87 (d, 1H, J=7.7 Hz), 8.20 (d, 1H, J=7.1 Hz).

### Example 43

### 2-(2-(Dimethylaminomethyl)phenylthio)-5-fluoromethylphenylamine

To a solution of intermediate 30 (138 mg, 0.43 mmol) in MeOH (10 mL) was added concentrated HCl (1.6 mL). The suspension was cooled to 0° C., SnCl₂ (247 mg, 1.30 mmol) was added and the reaction mixture stirred overnight at room temperature under nitrogen. The mixture was then diluted with H₂O (20 mL), and extracted with EtOAc (15 mL×2). The organic layers were discarded. The aqueous layer was adjusted to pH 10 with 1 N NaOH and extracted with EtOAc (25 mL×4). The combined organic layers were washed once with H₂O, dried, and concentrated. The crude product was chromatographed on silica gel to give the title compound (67.7 mg, 54%) as colorless oil. ¹H NMR: δ 7.51 (d, 1H, J=7.8 Hz), 7.30-7.24 (m, 1H), 7.16-7.07 (m, 2H), 6.95-6.90 (m, 1H), 6.77 (s, 1H), 6.74 (d, 1H, J=7.8 Hz), 5.32 (d, 2H, J=47.6 Hz), 4.76 (br s, 2H), 3.69 (s, 2H), 2.32 (s, 6H). An analytical sample was prepared by reaction of the title compound with equal moles of (dl)-tartaric acid in acetone to give the tartrate salt as a colorless solid, mp 142-143° C. Anal. Calcd for C₁₆H₁₉FN₂S.C₄H₆O₆: C, 54.53; H, 5.72; N, 6.36. Found: C, 54.24; H, 5.67; N, 6.23.

### Example 44

### 5-Fluoromethyl-2-(2-(methylaminomethyl)phenylthio)phenylamine

In an analogous manner the title compound was obtained from intermediate 31 in 53% yield as colorless oil. ¹H NMR: δ 7.45-7.30 (m, 2H), 7.18-7.00 (m, 2H), 6.82 (dd, 1H, J=1.5, 7.7 Hz), 6.76 (s, 1H), 6.70 (d, 1H, J=7.7 Hz), 5.30 (d, 2H, J=47.5 Hz), 3.97 (s, 2H), 2.51 (s, 3H). HRMS: calcd for C₁₅H₁₈FN₂S m/z (MH): 277.1175; found: 277.1171. Anal. Calcd for C₁₅H₁₇FN₂S: C, 65.19; H, 6.20; N, 10.14. Found: C, 64.72; H, 6.20; N, 9.94.

### Example 45

### 2-(2-(Dimethylaminomethyl)phenylthio)-5-(2-fluoroethyl)phenylamine

In an analogous manner the title compound was obtained from intermediate 32 in 77.4% yield as colorless oil. ¹H NMR: δ 2.31 (s, 6H) 2.95 (m, 2H), 3.58 (s, 2H), 4.57 (dt, 2H, J=4.9, 51.9 Hz), 6.59-7.40 (m, 6H), 7.42 (d, 1H, J=8.3 Hz).

### Example 46

### 5-Fluoroethyl-2-(2-(methylaminomethyl)phenylthio)-phenylamine

In an analogous manner the title compound was obtained from intermediate 33 in 55% yield as colorless oil. ¹H NMR: δ 2.52 (d, 3H, J=2.6 Hz), 2.99 (m, 2H), 3.74 (d, 2H, J=3.7 Hz), 3.94 (s, 2H), 4.60 (t, 1H, J=6.3 Hz), 4.71 (t, 1H, J=6.3 Hz), 6.59-6.71 (m, 2H), 6.83 (m, 1H), 7.11 (m, 2H), 7.29-7.38 (m, 2H).

### Example 47

### 2-(2-(Dimethylaminomethyl)phenylthio)-5-(3-fluoropropyl)-phenylamine

In an analogous manner the title compound was obtained intermediate 34 in 78.6% yield as colorless oil. ¹H NMR: δ 1.93-2.06 (m, 2H), 2.19 (s, 6H) 2.77 (t, 2H, J=7.9 Hz), 3.53 (s, 2H), 4.47 (dt, 2H, J=5.9, 53.2 Hz), 6.57-7.25 (m, 6H), 7.39 (d, 1H, J=8.3 Hz). An analytical sample was prepared by reaction of the title compound with (d,l)-tartaric acid to generate the tartrate salt as a colorless solid. Anal. Calcd for C₁₈H₂₃FN₂S.C₄H₆O₆: C, 56.40; H, 6.24; N, 5.98. Found: C, 56.13; H, 6.22; N, 5.89.

Preparation of Radiolabeled Compounds

General: Instruments used for radiochemistry are as follows: a semi-preparative HPLC system including a Waters 515 HPLC pump, a Rheodyne 7010 injector with a 2 mL loop, a Phenomenex Prodigy C-180DS-Prep column (10 μm, 10×250 mm), an Alltech Model 450 UV detector, a custom-made gamma detector, and a PC running LookOut HPLC data acquisition software; an analytical HPLC system consisting of a Waters 515 HPLC pump, a Rheodyne 7125 injector, a Phenomenex Prodigy C-180DS-3 column (5 μm, 4.6×250 mm), a Waters PDA 996 detector, a Flow Cell gamma detector (Bioscan) and a PC with Millenium software used for system control.

[¹¹C]CO₂ is produced via the ¹⁴N(p,α) ¹¹C nuclear reaction using a RDS112, 11 MeV negative-ion cyclotron. Routinely, the carbon-11 target (pressurized with 1% O₂ in N₂ to 200 psi) is irradiated with a beam current of 40 μA for 60 minutes to yield about 1300 mCi of [¹¹C] CO₂.

Anhydrous [¹⁸F]fluoride is prepared from aqueous [¹⁸F]NaF produced via the (p, n) nuclear reaction of [¹⁸O]H₂O in a RDS-112 cyclotron. To a 5 mL reaction vial containing equal moles of K₂CO₃ and Kryptofix-2.2.2., 500 to 600 μL of radioactivity will be added. The water is removed by repeated addition of anhydrous acetonitrile and azeotropic evaporation of the resulting mixture to bring the [¹⁸F]fluoride to complete dryness and ready for use in fluorination reaction. Irradiation of the target with a beam current of 30 μA for 60 minutes typically produces about 1000 mCi of [¹⁸F]fluoride.

Preparation of carbon-11 labeled compounds: Preparation of carbon-11 labeled compounds utilizes the general art of radiochemistry and is illustrated by the general procedure represented in Scheme 2:

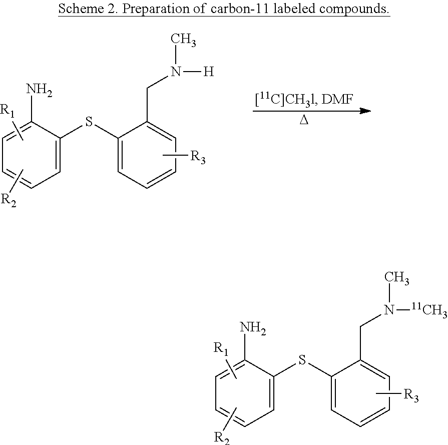

### Example 48

### [¹¹C]2-[2-(Dimethylaminomethyl)phenylthio]-5-methylphenylamine ([¹¹C]MADAM)

The radiolabeling precursor (example 39, 0.5-1.0 mg) was dissolved in N,N-dimethylformamide (DMF, 0.4 mL) in a 1.0 mL reaction vial. [¹¹C]Methyl iodide or triflate was bubbled through the precursor solution. When maximum radioactivity was reached in the vial, the bubbling and vent needles were removed and the reaction solution heated in a water bath (85-90° C.) for 5 minutes. The crude product was then purified using the semi-preparative HPLC system (Eluent: 30% MeCN/70% 0.1 M ammonium acetate solution; Flow rate: 10 mL/minute). The product fraction, eluted between 12-14 minutes, was collected, diluted with water (100 mL) and passed through a Waters classic C18 Sep-Pak. After washing with water (10 mL), the Sep-Pak was eluted with EtOH (1 mL) to recover the product. The EtOH solution was then mixed with saline, filtered through a 0.22 μm membrane filter and the filtered solution collected in a sterile vial. The title compound (10-100 mCi) was obtained in >93% chemical and radiochemical purity, as indicated by HPLC analysis of the ethanol solution (Eluent: 40% MeCN/60% 0.1 M ammonium formate; Flow rate: 2 mL/minute; Retention time for product: 5.8 minutes). Decay-corrected radiochemical yield was 25% based on [¹¹C]methyl iodide. Identity of the labeled compound was confirmed by co-injection of the product with the cold standard (example 38) onto the analytical HPLC. Radiolabeled product and the cold standard co-eluted on HPLC.

### Example 49

### [¹¹C]2-[2-(Dimethylaminomethyl)phenylthio]-5-fluorophenylamine ([¹¹C]AFA)

In analogy to example 48, the title compound was prepared in >96% radiochemical and chemical purity from its precursor (example 41) and [¹¹C]methyl iodide. Decay-corrected radiochemical yield was 21.5±8.3% based on [methyl iodide ¹¹C] (n=4). Identity of the labeled compound was confirmed by co-injection of the product with the cold standard (example 40) onto the analytical HPLC. Radiolabeled product and the cold standard co-eluted on HPLC (Eluent: 35% MeCN/65% 0.1 M ammonium formate; Flow rate: 2 mL/minute; Retention time for product: 8.5 minutes).

### Example 50

### [¹¹C]2-[2-(Dimethylaminomethyl)phenylthio]-5-fluoromethylphenylamine ([¹¹C] AFM)

In analogy to example 48, the title compound was prepared in >95% chemical and radiochemical purity from its precursor (example 44) and [¹¹C]methyl iodide. Radiochemical yield was 12.3±8.1% based on [¹¹C]methyl iodide and specific activity was 1733±428 Ci/mmol at end of synthesis (EOS, n=14).

Identity of the labeled compound was confirmed by co-injection of the product with the cold standard (example 43) onto the analytical HPLC. Radiolabeled product and the cold standard co-eluted on HPLC (Eluent: 30% MeCN/70% 0.1 M ammonium formate; Flow rate: 2 mL/minute; Retention time for product: 11.5 minutes).

### Example 51

### [¹¹C]2-[2-(Dimethylaminomethyl)phenylthio]-5-fluoroethylphenylamine ([¹¹C]AFE)

In analogy to example 48, the title compound was prepared in >95% chemical and radiochemical purity from its precursor (example 46) and [¹¹C]methyl iodide. Radiochemical yield was ˜20%. Identity of the labeled compound was confirmed by co-injection of the product with the cold standard (example 45) onto the analytical HPLC. Radiolabeled product and the cold standard co-eluted on HPLC (Eluent: 35% MeCN/65% 0.1 M ammonium formate; Flow rate: 2 mL/minute; Retention time for product: 8.2 minutes).

### Preparation of Fluorine-18 Labeled Compounds

### Example 52

### [¹⁸F]2-[2-(Dimethylaminomethyl)phenylthio]-5-fluorophenylamine ([¹⁸F]AFA)

The precursor 2-(2,4-dinitrophenylthio)-N,N-dimethylbenzylamine (intermediate 23) in DMSO was reacted at 150° C. with anhydrous [¹⁸F]fluoride complexed with Kryptofix-222 for 20 minutes to effect fluorination. The reaction mixture was then diluted with water and purified by passing through a C-18 SepPak and washed with water. Crude product eluted with 1.5 mL of EtOH was then reacted at 80° C. for 10 minutes with the reducing agent SnCl₂ dissolved in concentrated HCl. Alternatively, the reduction reaction can be effected using a combination of SnCl₂ or Cu(OAc)₂ with NaBH₄ in EtOH. Reaction mixture was diluted with water, passed through a C-18 SepPak and eluted with water. The crude product was then purified by preparative HPLC. Fraction containing the [¹⁸F]AFA peak was collected, diluted with water and passed through a C18 SepPak. The SepPak was washed with water. Final product was eluted off the SepPak with EtOH (1 mL), and formulated by dilution with sterile normal saline (9 mL) and filtered through a sterile membrane filter (0.22 μm). Radiochemical purity of the final product was >98%. Identity of the labeled compound was confirmed by co-injection of the product with the cold standard (example 40) onto the analytical HPLC. Radiolabeled product and the cold standard co-eluted on HPLC (Eluent: 35% MeCN/65% 0.1 M ammonium formate; Flow rate: 2 mL/minute; Retention time for product: 8.5 minutes).

### Example 53

### [¹⁸F]2-[2-(Dimethylaminomethyl)phenylthio]-5-fluoromethylphenylamine ([¹⁸F]AFM)

The radiolabeling precursor (intermediate 35, 2 mg) in acetonitrile (MeCN) was reacted at 80° C. with anhydrous [¹⁸F]fluoride complexed with Kryptofix-222 for 10 minutes to effect fluorination. The reaction mixture was then diluted with water and purified by passing through a C-18 SepPak and washed with water. Crude product eluted off the SepPak with EtOH (1-1.5 mL) was then reacted at 80° C. for 10 minutes with the reducing agent SnCl₂ dissolved in concentrated HCl. Alternatively, the reduction reaction can be effected using a combination of SnCl₂ or Cu(OAc)₂ with NaBH₄ in EtOH. Reaction mixture was diluted with water, passed through a C-18 SepPak and eluted sequentially, with 10% EtOH and water. The crude product was then purified by preparative HPLC. Fraction containing the [¹⁸F]AFM peak was collected, diluted with water and passed through a C18 SepPak. The SepPak was washed sequentially with 10% EtOH and water. Final product was eluted off with 1 mL of EtOH, and formulated by dilution with sterile normal saline (9 mL) and filtered through a sterile membrane filter (0.22 μm). Radiochemical purity of the final product was >95%. Identity of the labeled compound was confirmed by co-injection of the product with the cold standard (example 43) onto the analytical HPLC. Radiolabeled product and the cold standard co-eluted on HPLC (Eluent: 30% MeCN/70% 0.1 M ammonium formate; Flow rate: 2 mL/minute; Retention time for product: 11.5 minutes).

### Example 54

### [¹⁸F]2-[2-(Dimethylaminomethyl)phenylthio]-5-(2-fluoroethyl)phenylamine ([¹⁸F]AFE)

In analogy to example 53, the title compound was prepared from the radiolabeling precursor (intermediate 36) in >98% radiochemical purity. Radiochemical yield was >15%. Radiolabeled product and the cold standard (example 45) co-eluted on HPLC (Eluent: 40% MeCN/60% 0.1 M ammonium formate; Flow rate: 2 mL/minute; Retention time for product: 5.5 minutes).

### Example 55

### [¹⁸F]2-[2-(Dimethylaminomethyl)phenylthio]-5-(2-fluoropropyl)phenylamine ([¹⁸F]AFP)

In analogy to example 53, the title compound was prepared from the radiolabeling precursor (intermediate 37) in >95% radiochemical purity. Radiochemical yield was >15%. Radiolabeled product and the cold standard (example 47) co-eluted on HPLC (Eluent: 40% MeCN/60% 0.1 M ammonium formate; Flow rate: 2 mL/minute; Retention time for product: 7.5 minutes).

While ¹¹C and ¹⁸F have been emphasized herein as being particularly useful for PET analysis, other uses are contemplated including those flowing from physiological or pharmacological properties of stable isotope homologues and is apparent to those skilled in the art.

### II. Biological Testing

### Experiment 1

### In Vitro Binding Affinities of Candidate Radioligands for Imaging SERT In Vivo

### Abbreviations

McN 5652: known compound, (+)-6β-(4-methylthiophenyl)-1,2,3,5,6α,10β-hexahydropyrrolo[2,1-a]isoquinoline
ADAM: known compound, 2-{2-[(dimethylamino)methyl]phenylthio}-5-iodophenylamine
DASB: known compound, 3-amino-4-{2-[(dimethylamino)methyl]phenylthio}benzonitrile
MADAM: 2-[2-(dimethylaminomethyl)phenylthio]-5-methylphenylamine
AFA: 2-[2-(dimethylaminomethyl)phenylthio]-5-fluorophenylamine
AFM: 2-[2-(dimethylaminomethyl)phenylthio]-5-fluoromethyl phenylamine
AFE: 2-[2-(dimethylaminomethyl)phenylthio]-5-(2-fluoroethyl)phenylamine
AFP: 2-(2-(dimethylaminonethyl)phenylthio)-5-(3-fluoropropyl)phenylamine

Selected compounds were assayed in vitro for their binding affinity for the dopamine transporter (DAT), serotonin transporter (SERT), and norepinephrine transporter (NET) using membranes prepared from either the rat cortical tissues or cells expressed with cloned-human transporters and radioligands. Radioligands used were [³H]paroxetine (SERT), [³H]nisoxetine (NET), and [³H]GBR12935 (DAT). The inhibition coefficients (Ki) of selected compounds are listed in Table 1.

TABLE 1
In vitro binding affinities of selected compounds

| | Ki (nM) | | |
|---|---|---|---|
| Compound | SERT | DAT | NET |
| McN 5652 | 0.33 | >10,000 | 7.22 |
| ADAM | 0.32 | >10,000 | 1352 |
| DASB | 0.87 | >10,000 | 5127 |
| MADAM | 1.19 | >10,000 | 320 |
| AFA | 1.46 | >10,000 | 142 |
| AFM | 0.23 | >10,000 | 664 |
| AFE | 1.88 | >10,000 | 946 |
| AFP | 3.58 | >10,000 | 505 |

It is apparent from Table 1. that representative compounds disclosed in this invention (AFA, AFM, AFE and AFP) all have high affinity for SERT and appropriate selectivity for SERT over NET and DAT. They are therefore suitable candidates for development as imaging agents for the SERT.

### Experiment 2

### Biodistribution of Labeled SERT Radioligands in Male Sprague-Dawley Rats

### A. Method for Biodistribution Studies in Rats

Appropriately labeled compounds were studied in animals to assess their binding characteristics in the brain. Procedures for these biodistribution studies are as follows. The labeled compound in saline was injected into groups of male Sprague-Dawley rats (3 rats for each group) via the tail vein and the rats were sacrificed by decapitation, following anesthesia with CO₂, at desired time points (for example 10, 30, 60 and 90 minutes after radioactivity injection). The brain regions (cerebellum, hippocampus, striatum, frontal cortex, thalamus and hypothalamus), along with samples of blood and part of the tail, were removed, weighed, and counted in a Packard Cobra II gamma counter. The percent injected dose (% ID) of the decay- and tail-corrected activity in the brain regions and blood were calculated based upon C-11 standards prepared from the injection solution, and the % ID/g were calculated using the tissue weights.

### B. Results

Results from biodistribution studies in rats are presented in Table 2. From Table 2 it can be seen that all labeled compounds demonstrated desired properties of an appropriate radioligand for labeling SERT in vivo: 1) the radioligand enters the animal brain easily, with ID %/g in general exceeding 0.5% at 10 minute post-injection; 2) over time, the radioactivity accumulates in SERT-rich brain regions such as thalamus and hypothalamus and generates a good region of interest over cerebellem activity ratio, an indication of specific binding; 3) radioactivity reaches a high point, then decreases from there over time, indicating appropriate clearance.

TABLE 2
Biodistribution of radioligands in male Sprague-
Dawley rats % ID/g.

| | | | | | | | |
|---|---|---|---|---|---|---|---|
| Time | | | Frontal | | | | |
| (min) | Blood | Cerebellum | Ctx. | Striatum | Hippocamp. | Hypothal. | Thalamus |

[¹¹C]MADAM

| | | | | | | | |
|---|---|---|---|---|---|---|---|
| 10 | 0.61 ± 0.15 | 1.04 ± 0.33 | 2.17 ± 0.78 | 1.57 ± 0.54 | 1.39 ± 0.29 | 1.93 ± 0.72 | 1.95 ± 0.61 |
| 30 | 0.26 ± 0.07 | 0.55 ± 0.04 | 1.78 ± 0.23 | 1.41 ± 0.21 | 1.14 ± 0.17 | 1.48 ± 0.22 | 1.66 ± 0.17 |
| 60 | 0.15 ± 0.04 | 0.27 ± 0.01 | 0.89 ± 0.07 | 0.81 ± 0.15 | 0.84 ± 0.27 | 1.39 ± 0.28 | 1.14 ± 0.18 |

[¹¹C]AFA

| | | | | | | | |
|---|---|---|---|---|---|---|---|
| 10 | 0.26 ± 0.10 | 0.35 ± 0.12 | 0.70 ± 0.25 | 0.54 ± 0.18 | 0.48 ± 0.17 | 0.55 ± 0.18 | 0.60 ± 0.24 |
| 30 | 0.08 ± 0.01 | 0.14 ± 0.02 | 0.49 ± 0.06 | 0.38 ± 0.14 | 0.34 ± 0.02 | 0.47 ± 0.06 | 0.44 ± 0.05 |
| 60 | 0.06 ± 0.01 | 0.09 ± 0.02 | 0.29 ± 0.05 | 0.26 ± 0.04 | 0.24 ± 0.03 | 0.42 ± 0.06 | 0.39 ± 0.04 |

[¹¹C]AFM

| | | | | | | | |
|---|---|---|---|---|---|---|---|
| 10 | 0.37 ± 0.28 | 0.40 ± 0.06 | 0.99 ± 0.25 | 0.72 ± 0.17 | 0.59 ± 0.14 | 0.75 ± 0.19 | 0.79 ± 0.15 |
| 30 | 0.28 ± 0.01 | 0.41 ± 0.11 | 1.31 ± 0.28 | 1.12 ± 0.21 | 0.95 ± 0.29 | 1.37 ± 0.48 | 1.18 ± 0.30 |
| 60 | 0.19 ± 0.03 | 0.21 ± 0.04 | 1.37 ± 0.10 | 1.02 ± 0.15 | 0.84 ± 0.10 | 1.27 ± 0.16 | 1.29 ± 0.22 |

[¹¹C]AFE

| | | | | | | | |
|---|---|---|---|---|---|---|---|
| 10 | 0.15 ± 0.03 | 0.31 ± 0.03 | 0.61 ± 0.07 | 0.58 ± 0.08 | 0.44 ± 0.04 | 0.61 ± 0.11 | 0.63 ± 0.08 |
| 30 | 0.07 ± 0.01 | 0.12 ± 0.00 | 0.39 ± 0.03 | 0.35 ± 0.05 | 0.31 ± 0.03 | 0.44 ± 0.04 | 0.44 ± 0.05 |
| 60 | 0.05 ± 0.01 | 0.05 ± 0.00 | 0.19 ± 0.02 | 0.19 ± 0.02 | 0.18 ± 0.02 | 0.29 ± 0.03 | 0.22 ± 0.01 |

[¹⁸F]AFA

| | | | | | | | |
|---|---|---|---|---|---|---|---|
| 10 | 0.27 ± 0.01 | 0.38 ± 0.09 | 0.81 ± 0.26 | 0.65 ± 0.18 | 0.54 ± 0.17 | 0.73 ± 0.26 | 0.72 ± 0.24 |
| 30 | 0.16 ± 0.01 | 0.20 ± 0.01 | 0.65 ± 0.08 | 0.58 ± 0.05 | 0.48 ± 0.02 | 0.66 ± 0.05 | 0.69 ± 0.03 |
| 60 | 0.09 ± 0.00 | 0.07 ± 0.01 | 0.28 ± 0.05 | 0.26 ± 0.02 | 0.23 ± 0.04 | 0.38 ± 0.03 | 0.34 ± 0.05 |
| 90 | 0.08 ± 0.01 | 0.05 ± 0.01 | 0.20 ± 0.02 | 0.19 ± 0.01 | 0.18 ± 0.01 | 0.30 ± 0.06 | 0.23 ± 0.05 |
| 120 | 0.07 ± 0.01 | 0.04 ± 0.00 | 0.11 ± 0.02 | 0.11 ± 0.02 | 0.10 ± 0.01 | 0.17 ± 0.01 | 0.16 ± 0.02 |

[¹⁸F]AFM

| | | | | | | | |
|---|---|---|---|---|---|---|---|
| 10 | 0.37 ± 0.04 | 0.33 ± 0.04 | 0.82 ± 0.14 | 0.69 ± 0.15 | 0.51 ± 0.07 | 0.70 ± 0.08 | 0.72 ± 0.13 |
| 30 | 0.28 ± 0.02 | 0.23 ± 0.03 | 0.99 ± 0.20 | 0.83 ± 0.12 | 0.64 ± 0.12 | 0.82 ± 0.16 | 0.88 ± 0.06 |
| 60 | 0.10 ± 0.04 | 0.13 ± 0.02 | 0.71 ± 0.18 | 0.57 ± 0.12 | 0.45 ± 0.09 | 0.71 ± 0.14 | 0.67 ± 0.15 |
| 90 | 0.08 ± 0.03 | 0.08 ± 0.01 | 0.52 ± 0.19 | 0.45 ± 0.17 | 0.36 ± 0.12 | 0.58 ± 0.13 | 0.50 ± 0.15 |
| 120 | 0.04 ± 0.00 | 0.07 ± 0.02 | 0.38 ± 0.09 | 0.42 ± 0.10 | 0.28 ± 0.05 | 0.55 ± 0.08 | 0.56 ± 0.10 |

[¹⁸F]AFE

| | | | | | | | |
|---|---|---|---|---|---|---|---|
| 10 | 0.25 ± 0.01 | 0.28 ± 0.02 | 0.62 ± 0.08 | 0.51 ± 0.05 | 0.38 ± 0.03 | 0.51 ± 0.05 | 0.58 ± 0.08 |
| 30 | 0.13 ± 0.03 | 0.13 ± 0.01 | 0.32 ± 0.01 | 0.33 ± 0.01 | 0.27 ± 0.04 | 0.41 ± 0.03 | 0.39 ± 0.01 |
| 60 | 0.07 ± 0.02 | 0.06 ± 0.01 | 0.19 ± 0.04 | 0.21 ± 0.05 | 0.17 ± 0.04 | 0.24 ± 0.04 | 0.27 ± 0.04 |
| 90 | 0.04 ± 0.01 | 0.03 ± 0.01 | 0.08 ± 0.01 | 0.10 ± 0.02 | 0.09 ± 0.01 | 0.14 ± 0.02 | 0.17 ± 0.02 |
| 120 | 0.02 ± 0.01 | 0.02 ± 0.00 | 0.06 ± 0.00 | 0.08 ± 0.01 | 0.07 ± 0.00 | 0.10 ± 0.01 | 0.13 ± 0.02 |

(mean ± SD, n = 3)

To further determine the in vivo binding specificity of the radioligands to the SERT, blocking studies were carried out. Specifically, groups of rats were pre-treated with the selective serotonin re-uptake inhibitor citalopram, the selective norepinephrine re-uptake inhibitor nisoxetine, or the selective dopamine re-uptake inhibitor GBR 12935 (2 mg/kg each, i.v.). Radioactivity was injected 10 minutes after drug treatment, and rats were sacrificed 45 to 60 minutes after radioactivity administration. Brain parts were dissected and radioactivity levels (ID %/g) in brain regions calculated as above. For all the test compounds, pre-treatment with citalopram reduced radioactivity levels in the striatum, frontal cortex, thalamus and hypothalamus, regions with high densities of SERT, by 50 to 80%, while radioactivity level in the cerebellum, a region with negligible amount of SERT, remained little changed. Furthermore, pretreatment with either nisoxetine or GBR 12935 did not change radioactivity levels in the brain significantly. These data indicated that binding of these radiolabeled compounds in the rat brain is specific to the SERT, further demonstrating their usefulness as radioligands for the in vivo labeling of SERT.

### Experiment 3

### Characteristics of ¹¹C Labeled PET Radioligands for Imaging SERT In Vivo in the Baboon Brain

Imaging studies in baboons have been carried out to characterize selected C-11 labeled PET radioligands and compare their in vitro and in vivo pharmacological and pharmacokinetic properties. Radioligands included in these studies are [¹¹C]McN 5652, [¹¹C]ADAM, [¹¹C]DASB, [¹¹C]DAPA and [¹¹C]AFM. Details of the studies are described below.

### Abbreviations

McN 5652: (+)-6β-(4-methylthiophenyl)-1,2,3,5,6α,10β-hexahydropyrrolo[2,1-a]isoquinoline
ADAM: 2-{2-[(dimethylamino)methyl]phenylthio}-5-iodophenylamine
DASB: 3-amino-4-{2[(dimethylamino)methyl]phenylthio}benzonitrile
DAPA: 5-bromo-2-{2[(dimethylamino)methyl]phenylthio}phenylamine
AFM: 2-{2-[(dimethylamino)methyl]phenylthio}-5-fluoromethylphenylamine

### A. Materials and Methods

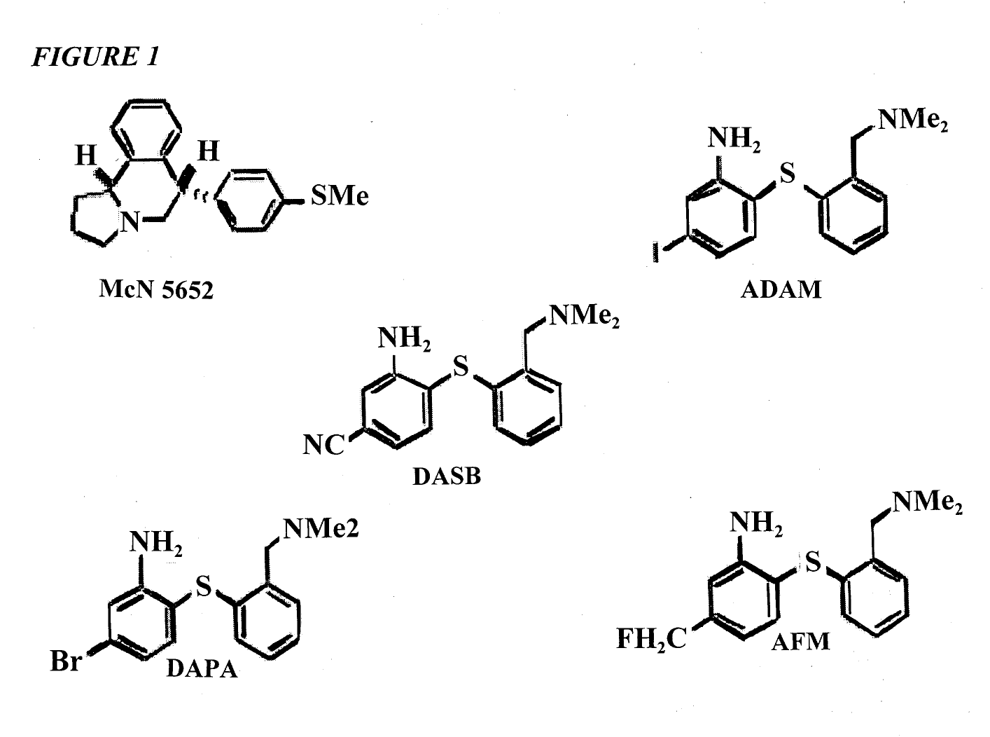

Chemistry: The standard and radiolabeling precursors of ADAM and DASB were synthesized at the University of Pennsylvania and the University of Toronto, respectively (Oya et al., 2000) (Wilson et al., 2000). The standard and radiolabeling precursors of McN 5652, DAPA and AFM were synthesized at Columbia University (Huang et al., 1998; Huang et al., 2001a; Huang et al., 2001b). Radiolabeling procedures were performed as previously described (Parsey et al., 2000; Wilson et al., 2000; Huang et al., 2001a; Huang et al., 2001b; Vercouillie et al., 2001). For the preparation of [¹¹C]ADAM, [¹¹C]DASB, [¹¹C]DAPA and [¹¹]AFM, the following procedure was used: radiolabeling precursor (0.3-0.5 mg) was dissolved in dimethylformamide (DMF, 0.4 mL) in a 1.0 mL reaction vial. [¹¹C]Methyl iodide, produced according to the literature procedure, was bubbled through the precursor solution. When maximum radioactivity was reached in the reaction vial, the bubbling and vent needles were removed and the reaction solution heated in a water bath (85-90° C.) for 5 minutes. The crude product was then purified using a semi-preparative HPLC system (HPLC column: Phenomenex Prodigy C180DS-Prep, 10μ, 10×250 mm; Eluent: mixture of MeCN and 0.1 M ammonium formate, pH 6.5, with composition depending on the particular tracer; Flow rate: 8-10 mL/minute). The product fraction was collected, diluted with water (100 mL) and passed through a Waters classic C18 Sep-Pak. After washing with water (10 mL), the Sep-Pak was eluted with EtOH (1 mL) to recover the radiolabeled product. The EtOH solution was then mixed with sterile normal saline (9 mL), filtered through a 0.22 μm membrane filter and the filtered solution collected in a sterile vial. The radiolabeled products (25-100 mCi) were obtained in >95% chemical and radiochemical purity. The structures of the unlabeled compounds listed above can be found in FIG. 1.

In vitro binding assays: Measurement of the inhibition constants (Kᵢ) of the labeled compounds in FIG. 1 on rat frontal cortex homogenates were performed at Columbia University using [³H]paroxetine, as previously described (Laruelle et al., 1988b). Briefly, male Sprague-Dawley rats (250-300 g) were killed by decapitation. Dissection of the brain was carried out on ice and samples of the frontal cortex were stored at −40° C. until binding assay. After thawing, samples were homogenized in 10 volumes of buffer (50 mM Tris-HCl, pH 7.4, 120 mM NaCl and 5 mM KCl). The homogenates were centrifuged at 10,000 rpm for 10 minutes. Pellets were resuspended in 10 volumes of buffer, incubated at 37° C. for 10 minutes, and centrifuged again. This procedure was then repeated a third time.

For the binding assay, membranes were suspended in buffer at a final protein concentration of 5 mg/mL. Unlabelled drugs were dissolved in dimethyl sulfoxide (DMSO) in borosilicate glass tubes to yield a 1 mM stock solution, from which subsequent serial dilutions were performed with the buffer. Aliquots of tissue preparation were incubated with [³H]paroxetine (0.1 nM) at 22° C. or 37° C. for 60 minutes in the absence or presence of unlabelled drug (10⁻⁵ to 10⁻¹² M) to yield a final volume of 0.5 mL (borosilicate glass tubes). Nonspecific binding was defined by incubation with 10 μM of citalopram. Each concentration was performed in triplicates. After incubation, the mixture was rapidly filtered through Whatman GF/B glass fiber filters using a 48-channel cell harvester (Brandel, Gaithersburg, Md.). The filters were washed 3 times with 4 mL each of ice cold buffer, placed in vials with 4 mL Ultima Gold solvent (Packard Instrument Co., Downers Grove, Ill.), and counted the following day for radioactivity in a liquid scintillation counter (Tricarb 1500, Packard Instrument Co., Downers Grove, Ill.). This experiment was performed three times at 22° C. and 37° C., on three different days. Kᵢ values were derived using GraphPad Prism software (San Diego, Calif.).

In vitro logP measurement: Octanol/water partition coefficient of the radiolabeled compounds were measured at Columbia University, using the method of Wilson et al. (2001), with some modifications. Briefly, 300 to 500 μCi of radioligand in EtOH was added to a separatory funnel containing octanol (20 mL) and phosphate buffer (20 mL, pH 7.4). The mixture was shaken mechanically for 3 minutes and the layers were separated. The octanol layer (15 mL) was transferred to a second separatory funnel containing 15 mL of the phosphate buffer (pH 7.4). The mixture was shaken mechanically for 3 minutes and the layers were separated. This second octanol layer was then partitioned into four test tubes (2 mL each) and the phosphate buffer (2 mL each) added. The test tubes were vortexed for 10 minutes, and then centrifuged for 10 minutes at 1000 rpm to separate the layers. The octanol and aqueous phases (1.0 mL each) were transferred into counting tubes and counted with a gamma counter (Wallac 1480 Wizard 3M Automatic Gamma Counter, Perkin-Elmer, Boston, Mass.). The radioactivity counts were decay-corrected and the partition coefficient calculated as P=counts in octanol/counts in buffer. Eight separate measurements were preformed for each tracer.

PET imaging protocol: PET experiments (n=20) were performed at Columbia University. Two adult male baboons (baboon A and B) were studied twice with each of the 5 radioligands (4 experiments per radioligand). All experiments took place within a 9-month period. Baboons A and B were 31 kg and 21 kg in weight at the beginning of the experimental period and 28 and 22 kg at the end, respectively. Test and retest experiments with the same radioligand were obtained on different days, with an average interval of 117±53 days.

Experiments were performed according to protocols approved by the Columbia-Presbyterian Medical Center Institutional Animal Care and Use Committee. Fasted animals were immobilized with ketamine (10 mg/kg i.m.), and anesthetized with 1.8% isoflurane via an endotracheal tube. Vital signs were monitored every 10 minutes and the temperature was kept constant at 37° C. with heated water blankets. An i.v. perfusion line was used for hydration and injection of radioligands and nonradioactive drugs. A catheter was inserted in a femoral artery for arterial blood sampling. Head was positioned at the center of the field of view as defined by imbedded laser lines. PET imaging was performed with the ECAT EXACT HR+PET scanner (Siemens/CTI, Knoxville, Tenn.). In 3D mode, this camera provides an in-plane resolution of 4.3 mm, 4.5 mm, 5.4 mm and 8.0 mm full width at half maximum (FWHM) at a distance of 0, 1, 10 and 20 cm from the center of the field of view, respectively (Brix et al., 1997). A 15 minute transmission scan was obtained prior to radioligand injection for attenuation correction. Activity was injected i.v. over 30 seconds. The injected mass and radioactivity had an upper limit of 6 μg and 6 mCi, respectively. Emission data was collected in the 3D mode for 91 minutes as 21 successive frames of increasing duration (6*10 seconds, 2*1 minutes, 4*2 minutes, 2*5 minutes, and 7*10 minutes).

Input function measurements: Arterial samples were collected every 5 seconds with an automated system for the first two minutes and manually thereafter at various intervals. A total of 28 samples were collected. Following centrifugation (10 minutes at 1,800 g), plasma was collected and activity measured in 200 μl aliquots on a gamma counter (Wallace 1480 Wizard 3M Automatic Gamma Counter, Perkin-Elmer, Boston, Mass.).

Selected plasma samples (n=6 per study, collected at 2, 4, 12, 30, 60 and 90 minutes after radioligand administration) were further processed by extraction with methanol (MeOH) followed by HPLC analysis to measure the fraction of plasma activity representing unmetabolized parent compound. The parent fraction was measured as follows: plasma (0.5 mL) was pipetted into a centrifuge tube containing MeOH (1.0 mL). The content in the tube was mixed and centrifuged (3.5 minutes at 15,000 rpm). The liquid phase was separated from the precipitate. Activity in 0.1 mL of the liquid phase was counted and the rest was injected onto the HPLC. The HPLC eluate was fraction-collected in 6 counting tubes (4.0 mL each). The HPLC system consisted of a Waters 510 isocratic pump, a Rheodyne injector equipped with a 2 mL sample loop, a C18 analytical column (Phenomenex ODS-prep, 10 μm, 4.6×250 mm), a Bioscan Flow Cell gamma detector (Bioscan, Washington, D.C.), and a fraction collector. The column was eluted with a mixture of acetonitrile and aqueous 0.1 M ammonium formate (proportion depending on the tracer being analyzed) at a flow rate of 2 mL/minute. Before plasma sample analysis, the retention time of the parent tracer was established by injection of a few μCi of the tracer and detection of the peak using the Bioscan gamma detector.

The parent fraction was calculated as the ratio of activity in the fractions containing the parent to that of the total activity collected. A biexponential function was fitted to the six measured parent fractions, and used to interpolate values between and after the measurements. The smallest exponential of the fraction parent curve, λₚₐᵣ, was constrained to the difference between λ_{cer}, the terminal rate of washout of cerebellar activity, and λₜₒₜ, the smallest elimination rate constant of the total plasma (Abi-Dargham et al., 1999) (Abi-Dargham et al., 1999). The input function was then calculated as the product of total counts and interpolated fraction parent at each time point. The measured input function values were fitted to a sum of three exponentials, and the fitted values were used as input for the kinetic analyses. The clearance of the parent compound (C_{L}, L/hour) was calculated as the ratio of the injected dose to the area under the curve of the input function (Rowland and Tozer, 1989; Abi-Dargham et al., 1994). The initial distribution volume (V_{bol}, L) was calculated as the ratio of injected dose to peak plasma concentration.

For the determination of the plasma free fraction (f₁), triplicate 200 μL aliquots of plasma, separated from blood collected prior to tracer injection and spiked with the radioligand, were pipetted into ultrafiltration units (Amicon Centrifree, Millipore, Bedford, Mass.) and centrifuged at room temperature (20 minutes at 4000 rpm) (Gandelman et al., 1994). Plasma and ultrafiltrate activities were counted, and f₁ was calculated as the ratio of ultrafiltrate activity to total plasma activity. Triplicate aliquots of the radioligand in pH 7.4 Tris buffer were also processed to determine the filter retention of free tracer.

Image analysis: A Magnetic Resonance Image (MRI) of each baboon's brain was obtained for the purpose of identifying the regions of interest (ROI) (T1-weighted axial MRI sequence, acquired parallel to the anterior-posterior commissure, TR 34 msec, TE 5 msec, flip angle of 45 degrees, slice thickness is 1.5 mm, zero gap, matrix 1.5 mm×1 mm×1 mm voxels). The following regions were drawn on the MR images: cerebellum, midbrain, thalamus, striatum, hippocampus, temporal cortex, cingulate cortex and occipital cortex.

PET emission data were attenuation-corrected using the transmission scan, and frames were reconstructed using a Shepp filter (cutoff 0.5 cycles/projection rays). Reconstructed image files were then processed using the image analysis software MEDx (Sensor Systems, Inc., Sterling, Va.). An image was created by summing all the frames, and this summed image was used to define the registration parameters with the MR image, using between-modality automated image registration (AIR) algorithm (Woods et al., 1993). Registration parameters were then applied to the individual frames for registration to the MR data set. Regional boundaries were transferred to the individual registered PET frames, and time-activity curves were measured and decay-corrected. Right and left regions were averaged. For a given animal, the same regions were used for all radioligands.

Brain uptake: Total brain uptake was expressed as percentage of the injected dose per gram of tissue (% ID g⁻¹) in a region encompassing the entire brain. Regional peak time was defined as the mid-time of the frame associated with the highest activity value. To estimate the degree of wash-out from the brain captured during the scan interval (91 minutes), the decrease in activity from the peak frame to the last frame was calculated and expressed in percentage of the peak activity.

Kinetic analysis: Regional total distribution volumes (V_{T}, mL g⁻¹) were derived with kinetic analysis of the regional time-activity curves, using the arterial plasma concentrations as input function. For each tracer, the use of a two-tissue compartment model was associated with an unacceptable number of non-convergences or convergences with negative rate constants and with large error in the estimates of V_{T} (data not shown). In contrast, a one-tissue compartment model converged in all cases and generally provided good fit to the data. Therefore, the one-tissue compartment model was selected for this investigation. In the one-tissue compartment model, V_{T} is derived as the K₁/k₂ ratio, where K₁ (mL g⁻¹ min⁻¹) and k₂ (min⁻¹) are the unidirectional fractional rate constant for the transfer of the tracer in and out of the brain, respectively.

Kinetic parameters were derived by nonlinear regression using a Levenberg-Marquart least squares minimization procedure implemented in MATLAB (The Math Works, Inc., South Natick, Mass.) as previously described (Laruelle et al., 1994c). Given the unequal sampling over time (increasing frame acquisition time from the beginning to the end of the study), the least squares minimization procedure was weighted by the square root of the frame acquisition time. The contribution of plasma total activity to the regional activity was calculated assuming a 5% blood volume in the ROI and subtracted from the regional activity prior to analysis (Mintun et al., 1984). No correction was applied for the delay between the arrival of activity in the femoral artery and the brain.

The minimal scanning time required to achieve time-independent derivation of regional V_{T} was evaluated by fitting the time-activity curves to shorter data sets, representing total scanning time of 81, 71, 61, 51, and 41 min, respectively. The resulting estimates of V_{T} were normalized to the V_{T} derived with the 91 minute data set. For each region and each tracer (n=4 observations per scan duration), the average and standard deviation of the normalized V_{T} were calculated. Time independence was considered achieved when two criteria were fulfilled: 1) the average normalized V_{T} had is between 95% and 105% of the reference V_{T} (small bias), and 2) the SD of the normalized V_{T} is less than 10% (small error).

Derivation of SERT parameters: Because of the low to negligible levels of SERT in the cerebellum, cerebellar V_{T} was used as an estimate of the nonspecific distribution volume (including free and nonspecific binding) in the ROIs. Therefore, BP (mL g⁻¹) was derived as the difference between V_{T} in the ROI (V_{T} ROI) and V_{T} in the cerebellum (V_{T} CER). Under these conditions, BP is equal to (Laruelle et al., 1994b):

$$BP = {V}_{TROI} - {V}_{TCER} = \frac{{f}_{1} {B}_{max}}{{K}_{D}}$$

where Bₘₐₓ is the regional concentration of SERT (nM per g of tissue), and K_{D} is the in vivo affinity of the tracer for SERT (nM per mL of brain water).

For between tracer comparisons, the main outcome measure of interest was the specific to nonspecific equilibrium partition coefficient (V₃″). V₃″ was calculated as the ratio of BP to V_{T CER}. Under these conditions, V₃″ is equal to (Laruelle et al., 1994b):

$${V}_{3}^{″} = \frac{BP}{{V}_{TCER}} = \frac{{f}_{2} {B}_{max}}{{K}_{D}}$$

where f₂ is the free fraction in the nonspecific distribution volume of the brain (f₂=1/V_{T CER}). V₃″ provides an appropriate measure of the “signal to noise contrast” associated with the detection of specific binding, in that it is independent of between-tracer differences in plasma clearance and independent of the time of measurement.

The test/retest reproducibility of V₃″ was calculated as the absolute difference between test and retest normalized to the average of the test and retest.

Statistical analysis: Values are given as mean±SD. Dependent variables were analyzed using ANOVA or repeated measures ANOVA, when appropriate. Post-hoc test were performed using Fisher PLSD. A two tailed probability value of 0.05 was selected as the significance level.

### B. Results

In Vitro Experiments

Affinity: Table 3 lists the Kᵢ values of the radioligands at 22° C. and 37° C. Statistically significant differences in Kᵢ were noted between ligands (p<0.0001) and temperatures (p=0.007), and a significant ligand by temperature interaction was noted (p=0.0038). At post-hoc contrasts, DASB Kᵢ was significantly higher than the four other compounds (p<0.05 for all contrasts). The Kᵢ of McN 5652, ADAM, DAPA and AFM did not significantly differ from each other. Temperature had no significant effect on the Kᵢ of McN 5652, ADAM, DAPA and AFM, but significantly decreased the affinity of DASB for SERT (p=0.04).

TABLE 3
Affinities for SERT and lipophilicities.

| | Ki (nM) | | | |
|---|---|---|---|---|
| Ligand | at 22° C. | at 37° C. | p | Log P |
| McN 5652 | 0.33 ± 0.12 | 0.26 ± 0.13 | 0.30 | 2.51 ± 0.03 |
| ADAM | 0.32 ± 0.11 | 0.40 ± 0.16 | 0.41 | 3.31 ± 0.13 |
| DASB | 0.87 ± 0.41 | 1.77 ± 0.25 | 0.04 | 2.38 ± 0.03 |
| DAPA | 0.38 ± 0.24 | 0.33 ± 0.13 | 0.85 | 3.06 ± 0.12 |
| AFM | 0.23 ± 0.04 | 0.42 ± 0.12 | 0.16 | 2.44 ± 0.03 |

Values are mean ± SD, n = 3 per radioligand for binding assays.
n = 8 per ligands for log P measurements.
p = significance level of the temperature effect, paired t test.

Lipophilicity: Significant between tracer differences were observed for logP (p<0.0001). Post-hoc tests showed that [¹¹C]DASB was significantly less lipophilic than [¹¹C]McN 5652, [¹¹C]ADAM and [¹¹C]DAPA, but not significantly different from [¹¹C]AFM (Table 3). [¹¹C]ADAM and [¹¹C]DAPA were significantly more lipophilic than the four other tracers.

In Vivo PET Experiments

### Injected Doses

Injected doses, specific activities at the time of injection and injected masses did not significantly differ between radioligands (p=0.82, 0.48 and 0.14, respectively, Table 4).

TABLE 4
Injected Doses.

| | | Specific activity | |
|---|---|---|---|
| | Injected dose | @ time of injection | Injected mass |
| Radiotracer | (mCi) | (Ci/mmol) | (μg) |
| [¹¹C] McN 5652 | 4.95 ± 0.40 | 803 ± 135 | 1.88 ± 0.47 |
| [¹¹C] ADAM | 4.03 ± 1.63 | 597 ± 378 | 3.49 ± 2.01 |
| [¹¹C] DASB | 4.33 ± 0.96 | 1067 ± 322 | 1.21 ± 0.39 |
| [¹¹C] DAPA | 4.84 ± 0.69 | 1064 ± 622 | 2.33 ± 1.97 |
| [¹¹C] AFM | 3.84 ± 1.26 | 953 ± 499 | 1.32 ± 0.47 |

Values are mean ± SD of four experiments.

### Plasma Analysis

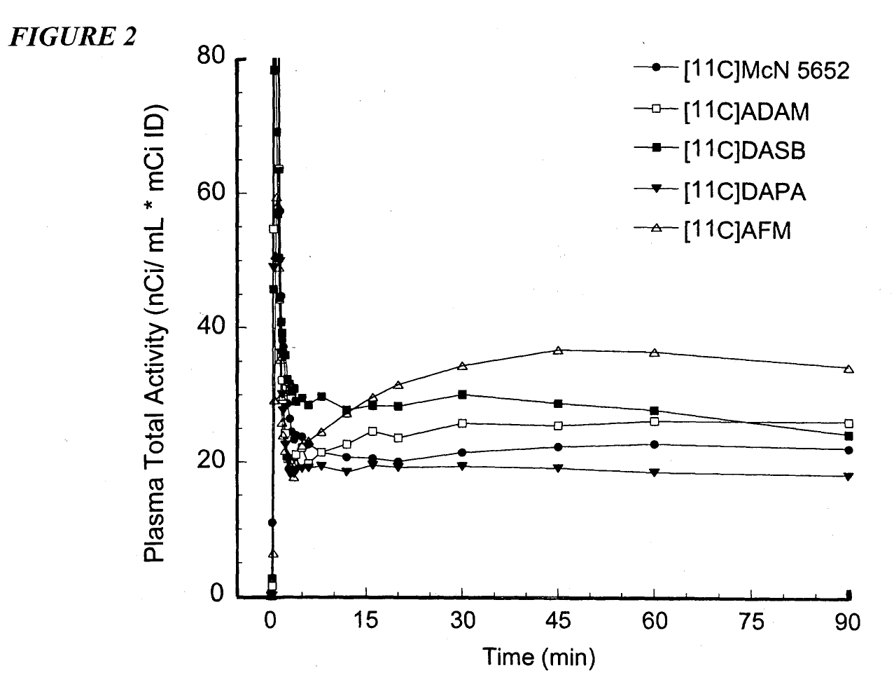

Total activity: Following a rapid distribution phase, total plasma activities stabilized at relatively constant levels (FIG. 2). After the initial distribution phase, [¹¹C]AFM and [¹¹C]ADAM total plasma activities increased, indicating that the distribution volumes of these two compounds were larger than those of their metabolites.

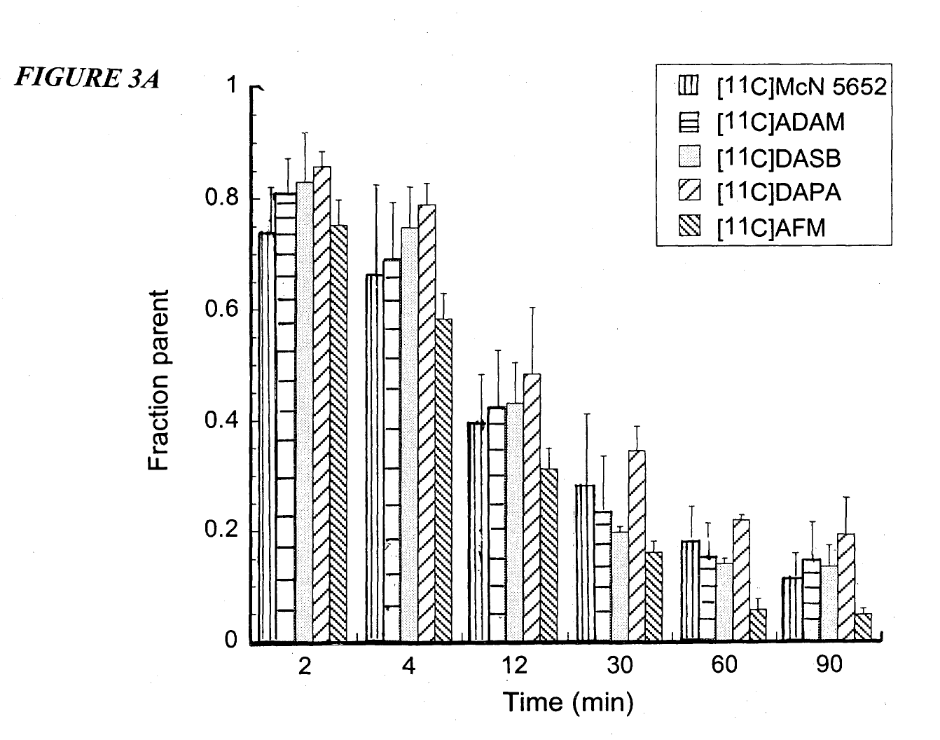

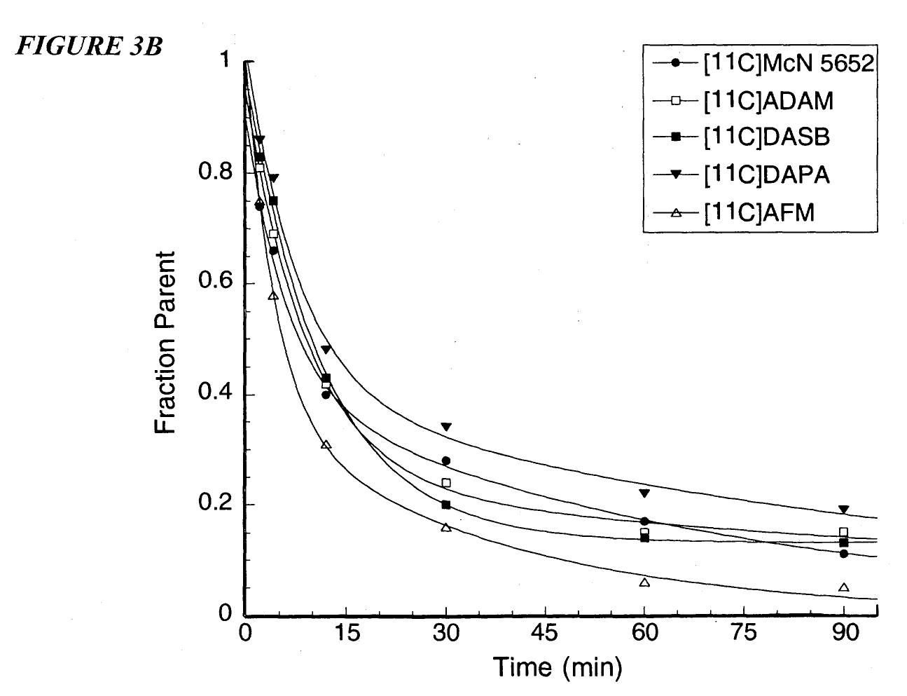

Metabolism: Tracers were metabolized into more polar compounds at a moderate rate. Plasma parent fraction as percentage of the total plasma activity measured at 2, 4, 12, 30, 60 and 90 min was analyzed with repeated measure ANOVA, with time as repeated measure and baboons and tracers as cofactors (FIGS. 3A and 3B). Statistically significant differences in rate of metabolism were noted between tracers (p=0.01), but not between animals (p=0.32). Post-hoc analysis revealed that [¹¹C]AFM had a significantly faster metabolism compared to [¹¹C]ADAM, [¹¹C]DASB and [¹¹C]DAPA.

Input function: Table 5 lists the initial distribution volumes (V_{bol}) and clearances of the parent compounds. Regarding V_{bol}, no between animal differences (p=0.74), but significant between tracer differences were observed (p=0.003). [¹¹C]AFM had significantly higher V_{bol} than [¹¹]DASB, and both [¹¹C]AFM and [¹¹]DASB V_{bol} were significantly higher than [¹¹C]McN 5652, [¹¹C]ADAM and [¹¹C]DAPA V_{bol}. Regarding the clearance of the parent compounds, significant differences were observed between baboons (p=0.003), and differences between radioligands were observed at trend level (p=0.08). Combining all tracers, baboon A showed faster clearance (85±30 L h⁻¹, n=10) than baboon B (56±14 L h⁻¹, p=0.008). The clearance of [¹¹C]AFM was faster than the clearance of [¹¹C]McN 5652 (p=0.03), [¹¹C]DAPA (p=0.03) and [¹¹C]ADAM (p=0.09), and similar to the clearance of [¹¹C]DASB (p=0.66).

Plasma free fraction: Table 5 lists the percent activity remaining on the filters following ultracentrifugation of the radioligands dissolved in saline, as well as the plasma free fraction (f₁). Because of previously demonstrated high retention of [¹¹C]McN 5652 on the filter (Parsey et al., 2000), [¹¹C]McN 5652 f₁ was not measured. This problem was also encountered with [¹¹C]ADAM and [¹¹C]DAPA where more than 75% of the activity remained on the filter following ultracentrifugation of these tracers dissolved in saline. In contrast, [¹¹C]DASB and [¹¹C]AFM showed moderated (<20%) filter retention when dissolved in saline. Thus, the measured f₁ approximates the in vivo f₁ only for these two tracers. [¹¹C]DASB f1 was significantly higher than [¹¹C]AFM f1(p=0.023).

TABLE 5
Plasma analysis of parent compound: Vbol, clearance,
retention on filter, and plasma free fraction.

| | Vbol | Clearance | Filter | f1 |
|---|---|---|---|---|
| Radiotracer | (L) | (L h⁻¹) | retention (%) | (%) |
| [¹¹C] McN | 5.9 ± 1.8 | 58 ± 13 | — | — |
| 5652 | | | | |
| [¹¹C] ADAM | 8.1 ± 3.1 | 67 ± 13 | 88.2 ± 5.2% | 0.9 ± 0.7% |
| [¹¹C] DASB | 12.6 ± 2.2 | 84 ± 23 | 19.2 ± 4.7% | 13.5 ± 1.7% |
| [¹¹C] DAPA | 8.5 ± 1.8 | 58 ± 22 | 77.9 ± 6.6% | 1.8 ± 0.5% |
| [¹¹C] AFM | 17.6 ± 1.3 | 90 ± 46 | 19.3 ± 4.1% | 9.6 ± 0.5% |

Values are mean ± SD of four experiments.

### Brain Analysis

Total Brain uptake: All tracers showed excellent brain penetration, with total brain uptake higher than 0.01% ID g⁻¹ at 45 minutes. Statistically significant differences in total brain uptake was noted between baboons (p=0.047) and ligands (p=0.015). Baboon A (the animal with the faster clearance) showed lower brain uptake (0.015±0.003% ID g⁻¹) than baboon B (0.017±0.001% ID g⁻¹) [¹¹C]DASB showed significantly lower total brain uptake at 45 min (0.013±0.002% ID g⁻¹) compared to [¹¹C]McN 5652 (0.016±0.002% ID g⁻¹, p=0.017), [¹¹C]ADAM (0.017±0.001% ID g⁻¹, p=0.011), [¹¹C]DAPA (0.018±0.002% ID g⁻¹, p=0.001), and [¹¹C]AFM (0.016±0.002% ID g⁻¹, p=0.027).

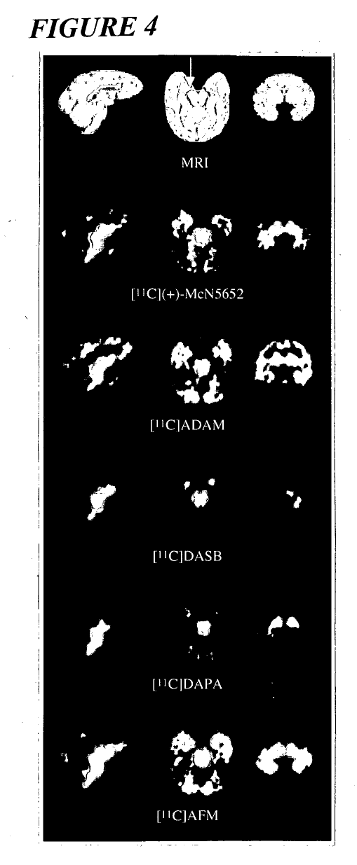

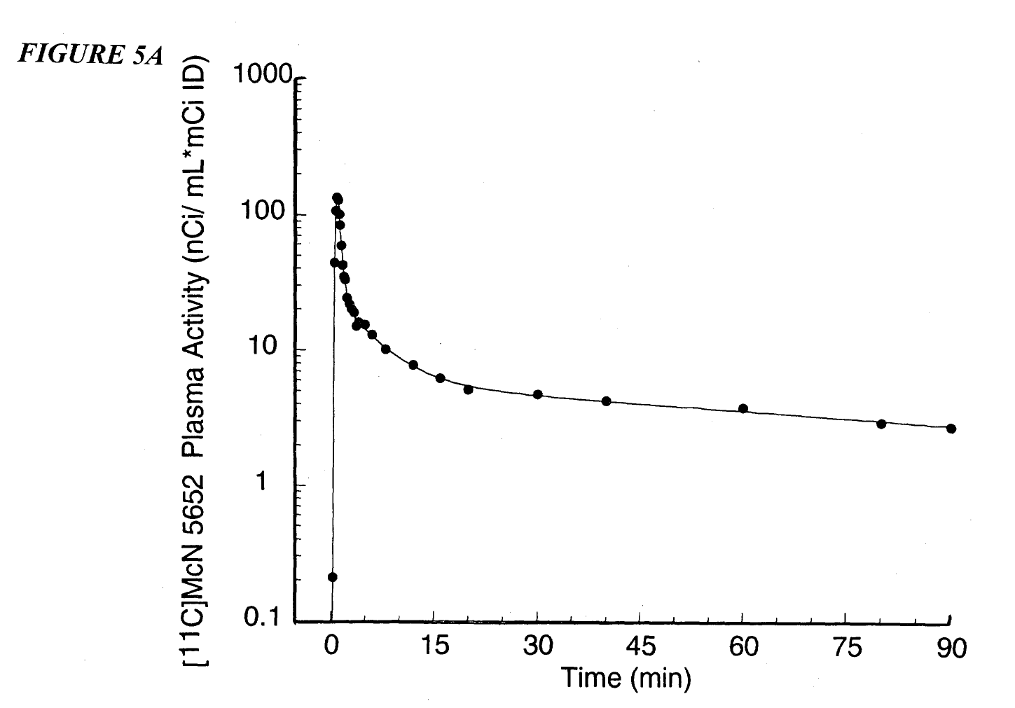

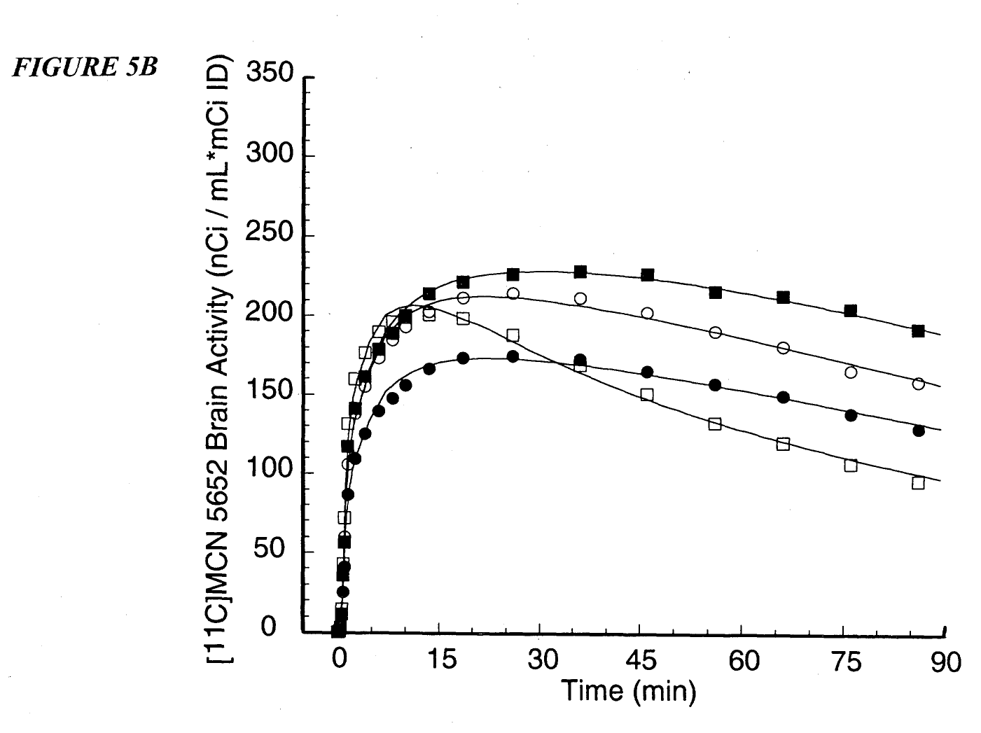

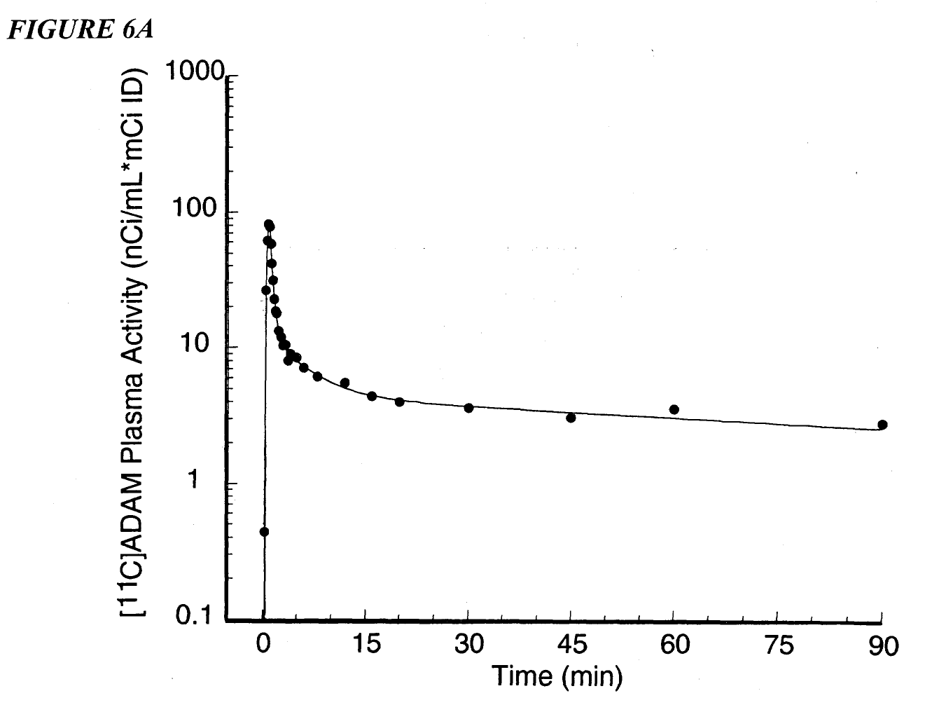

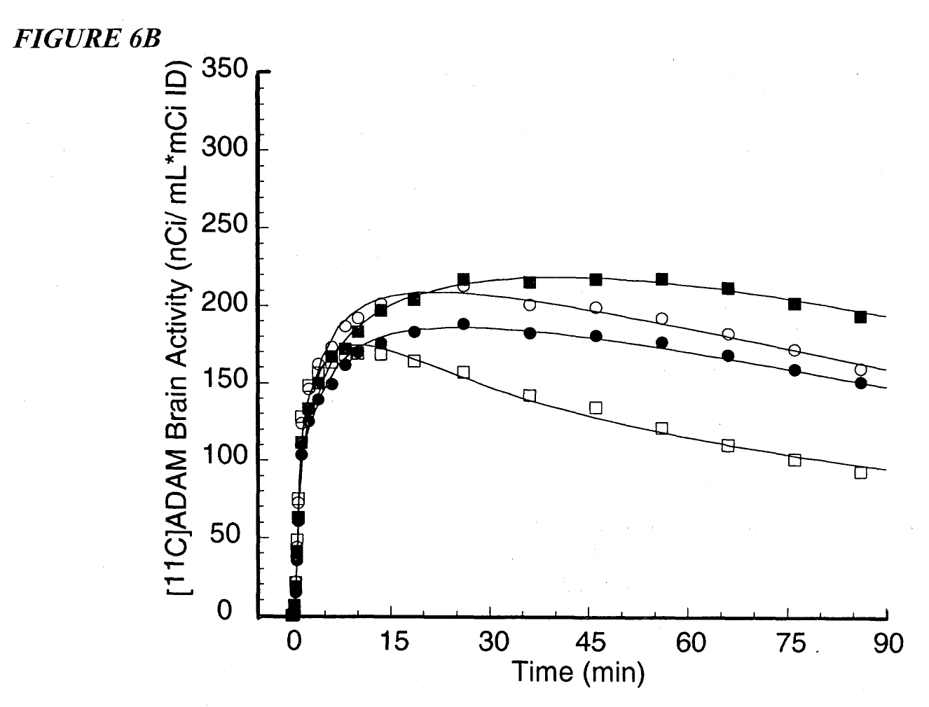

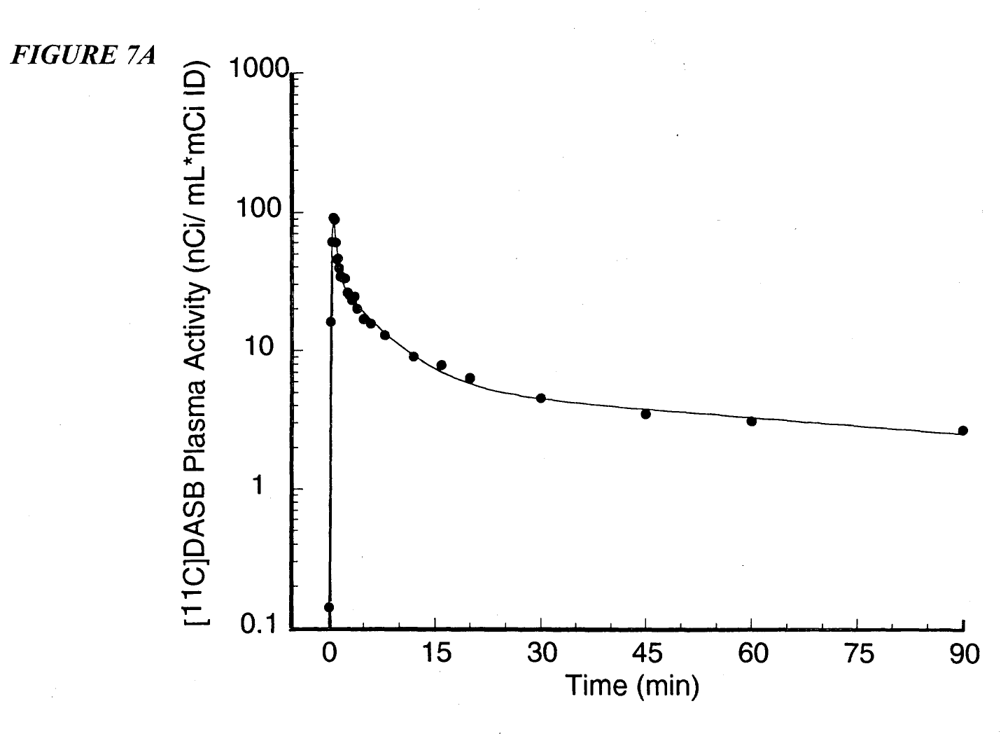

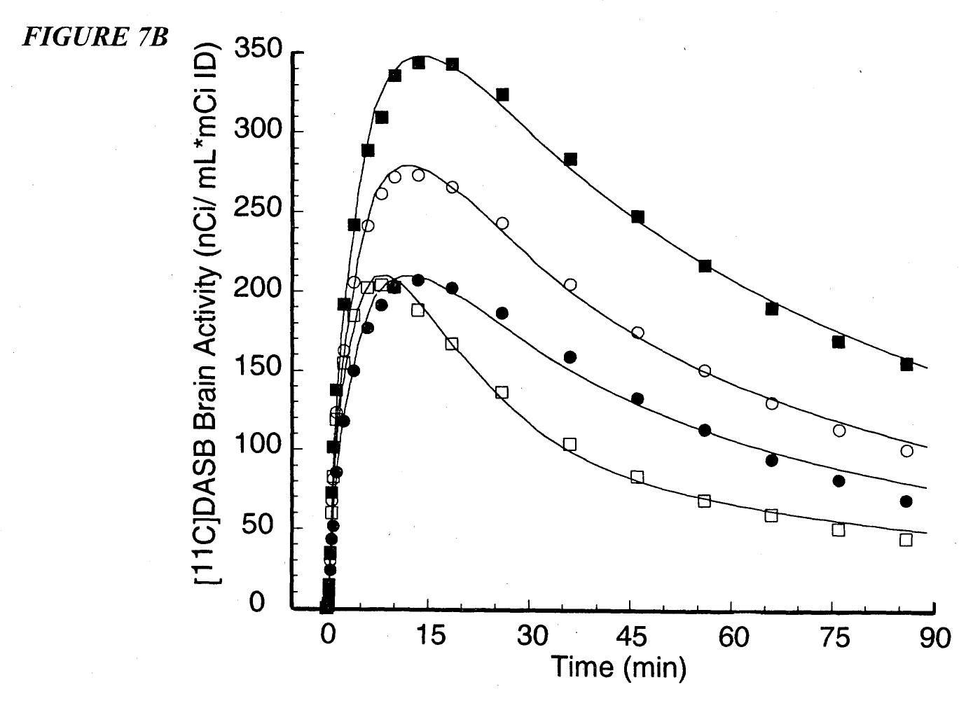

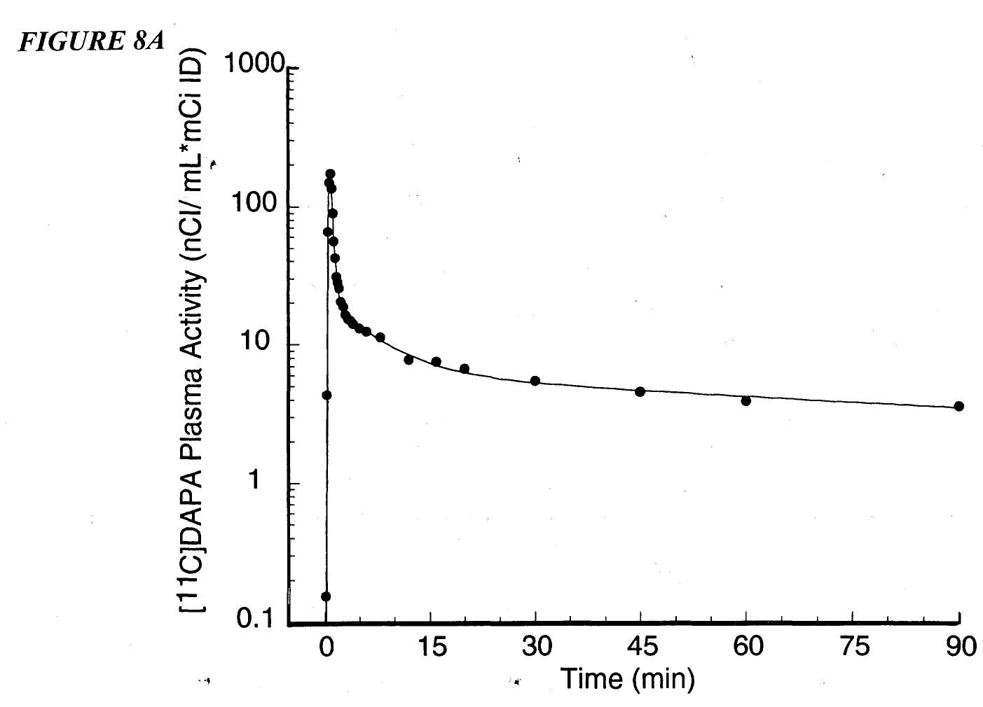

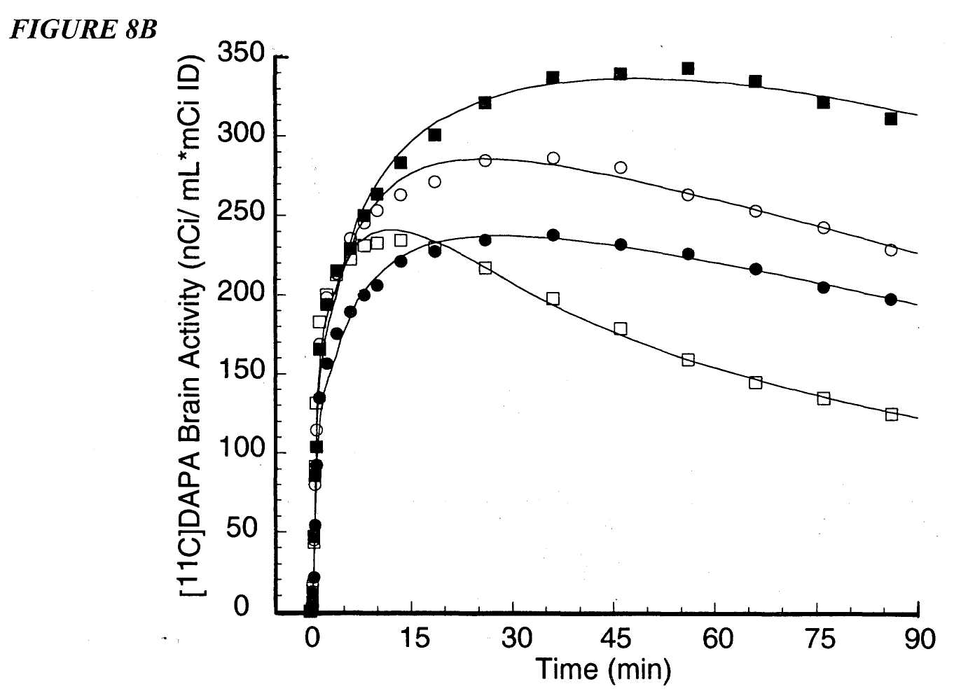

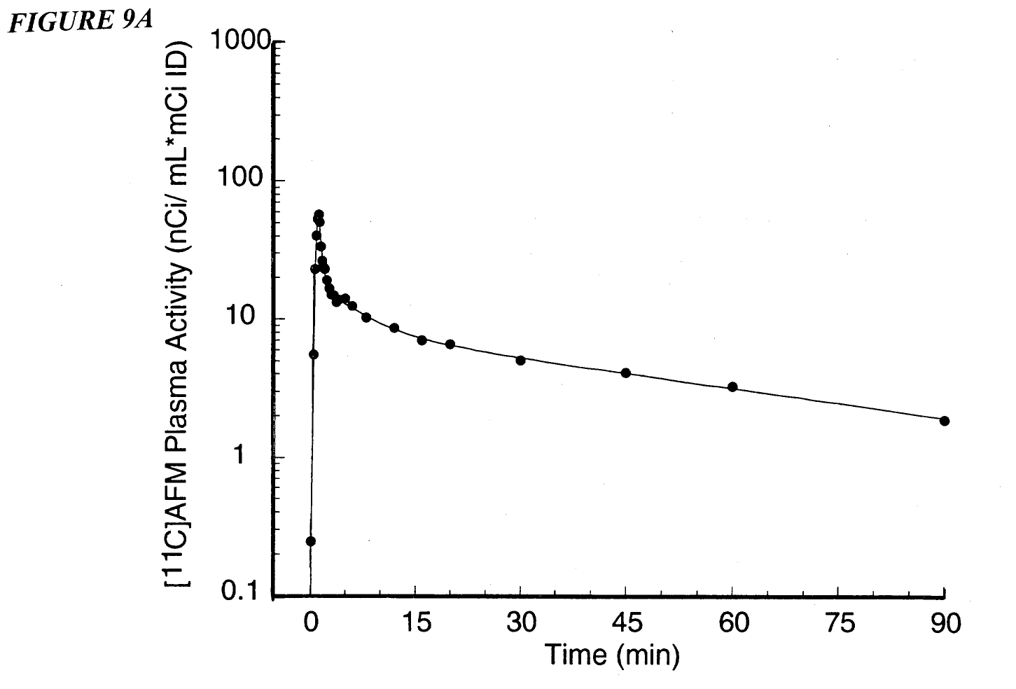

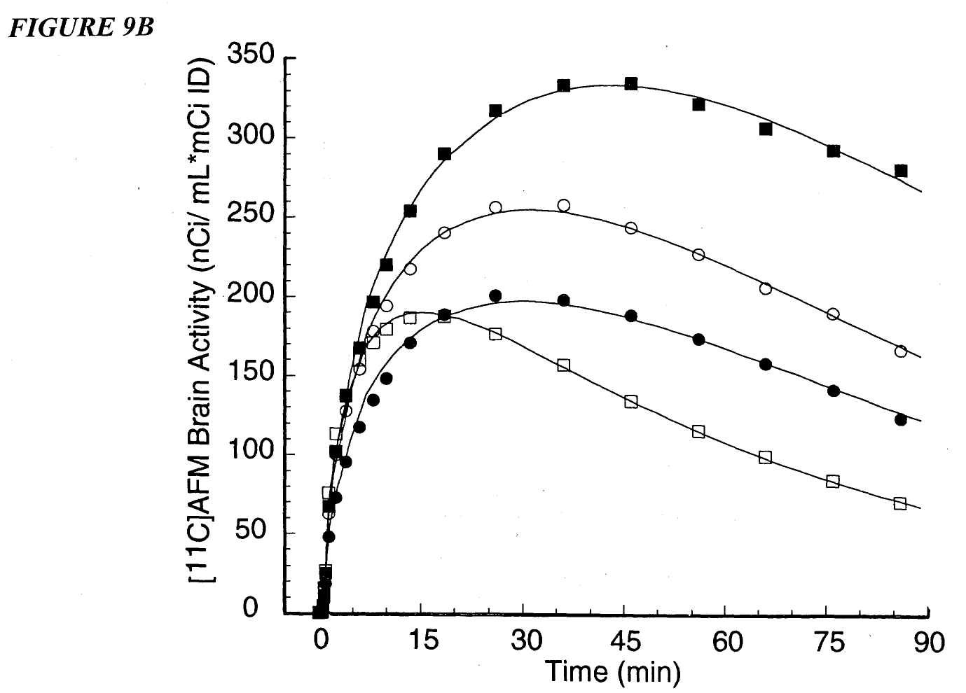

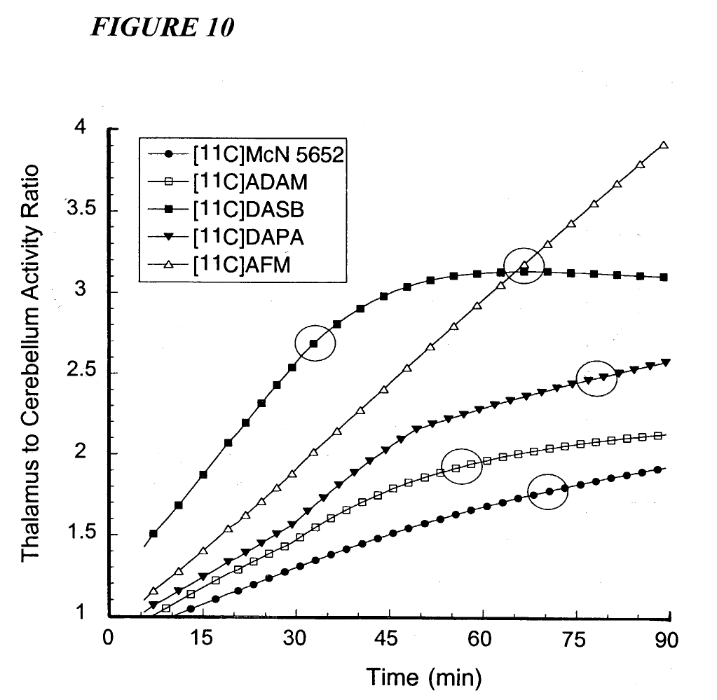

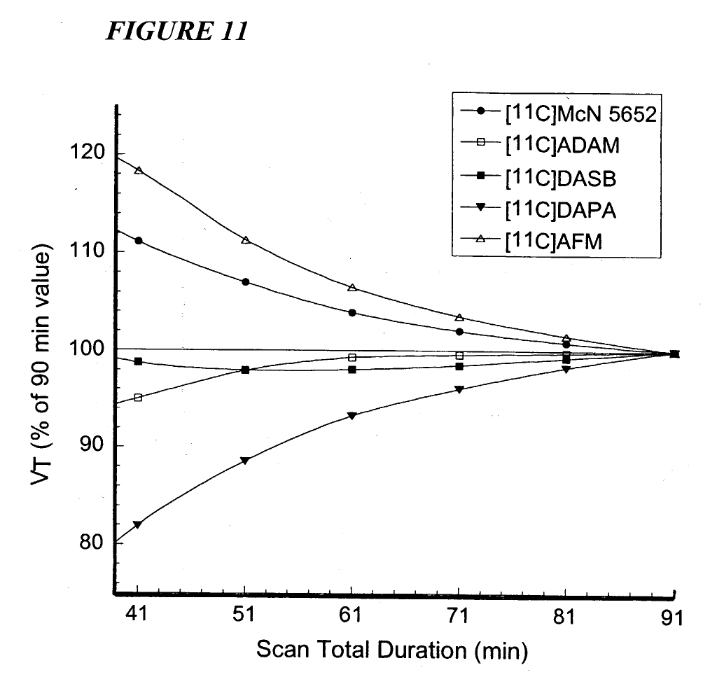

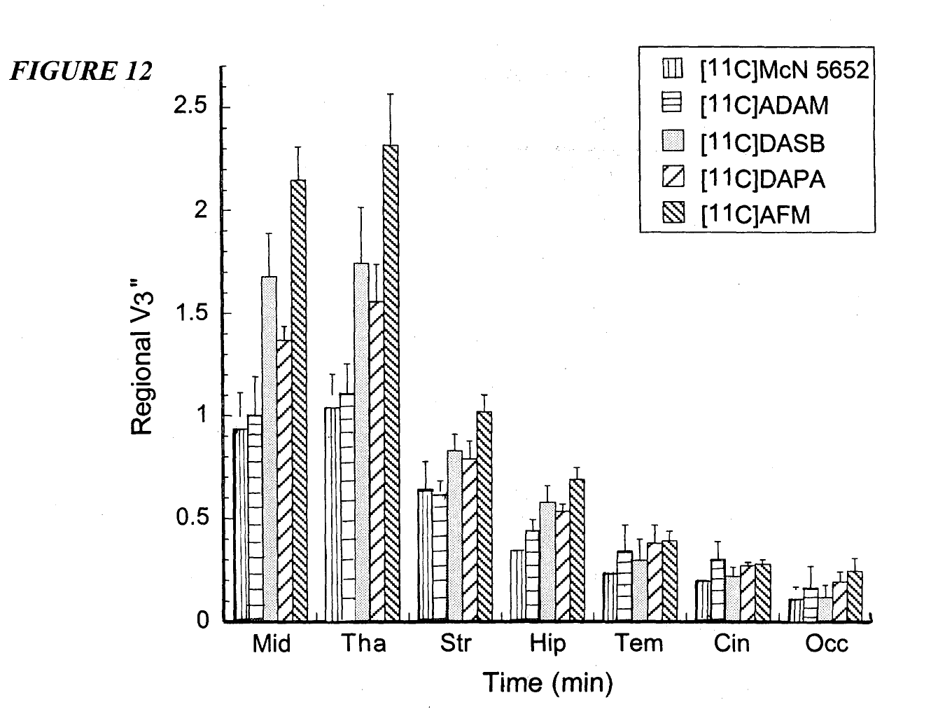

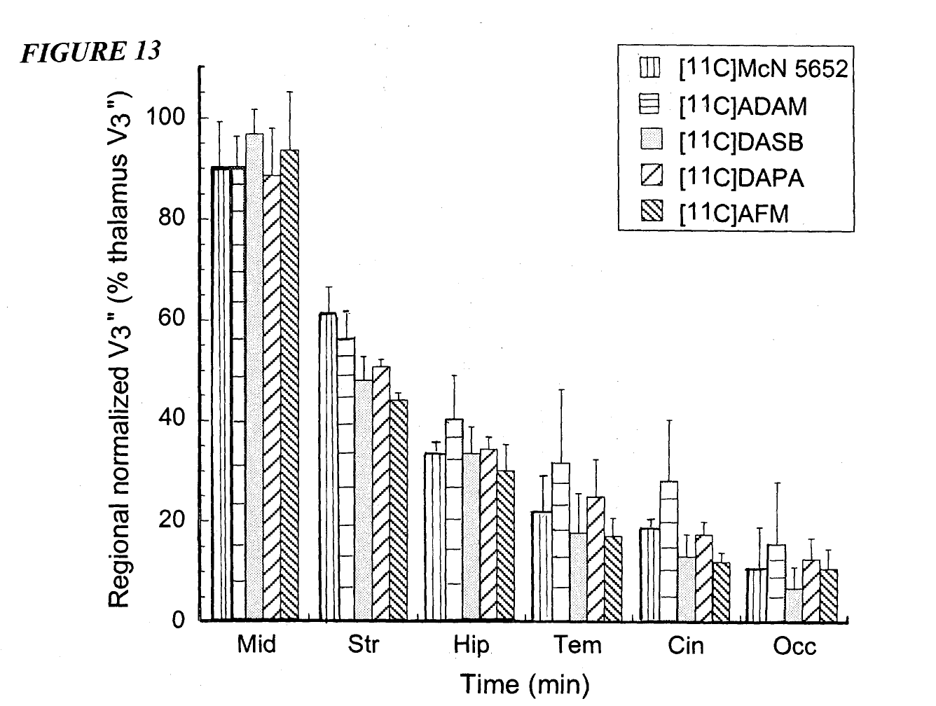

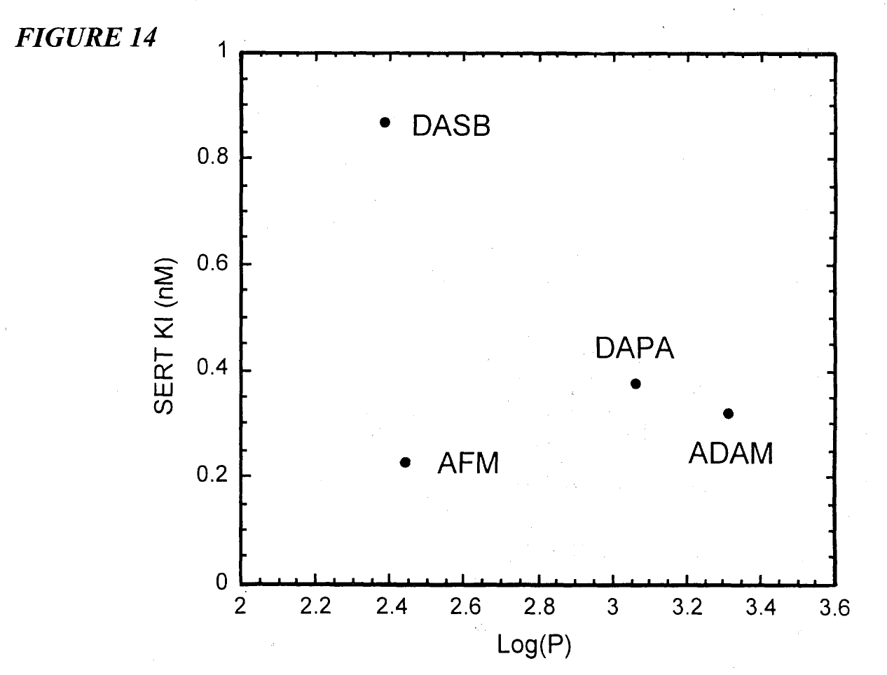

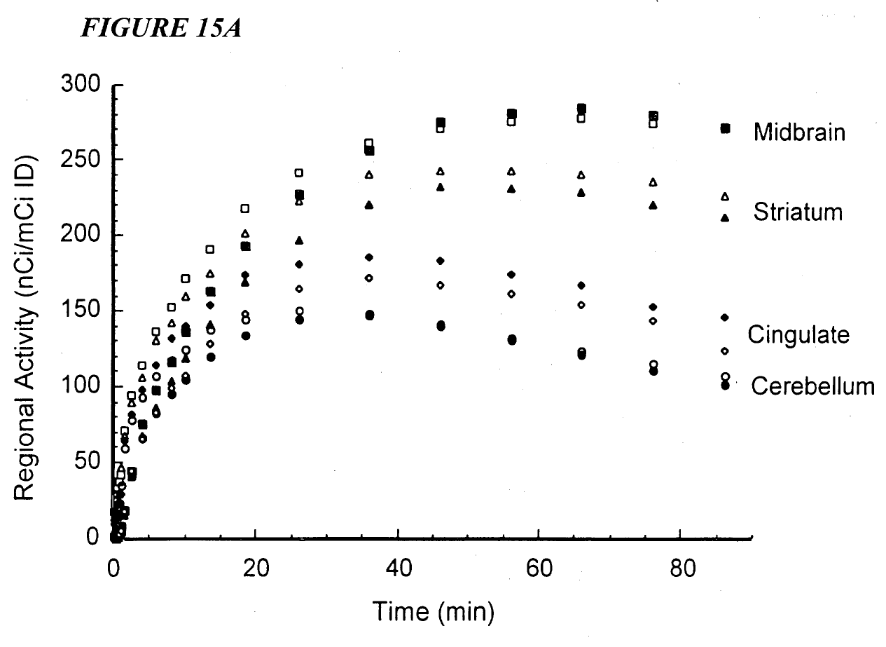

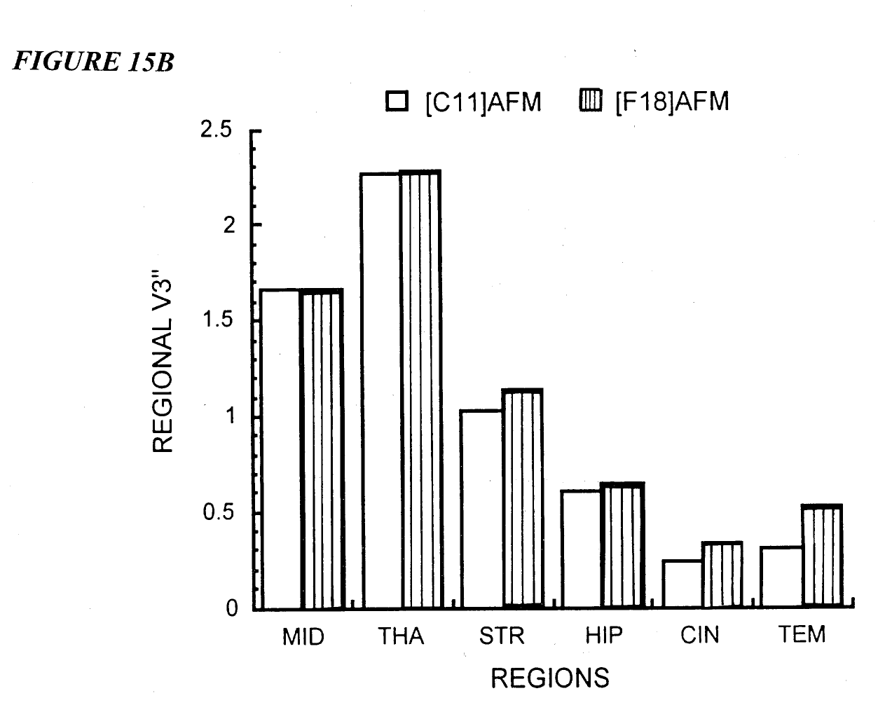

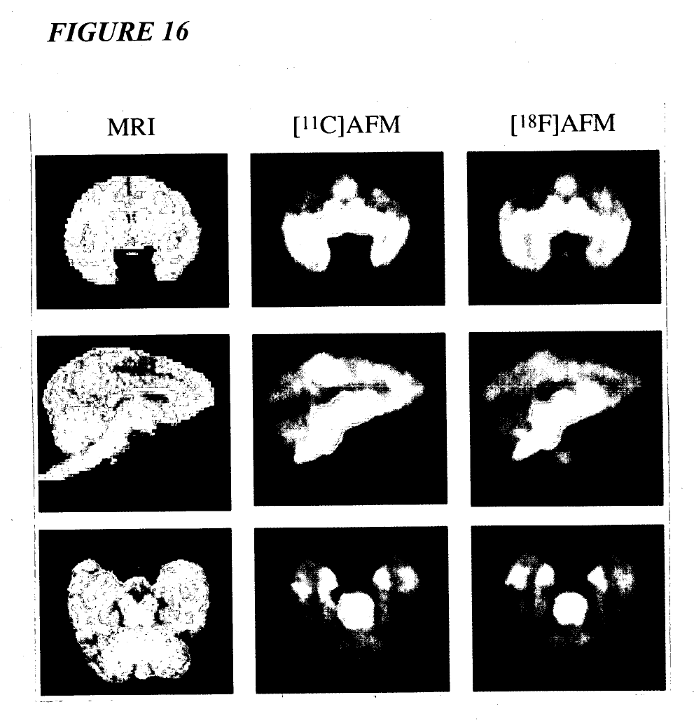

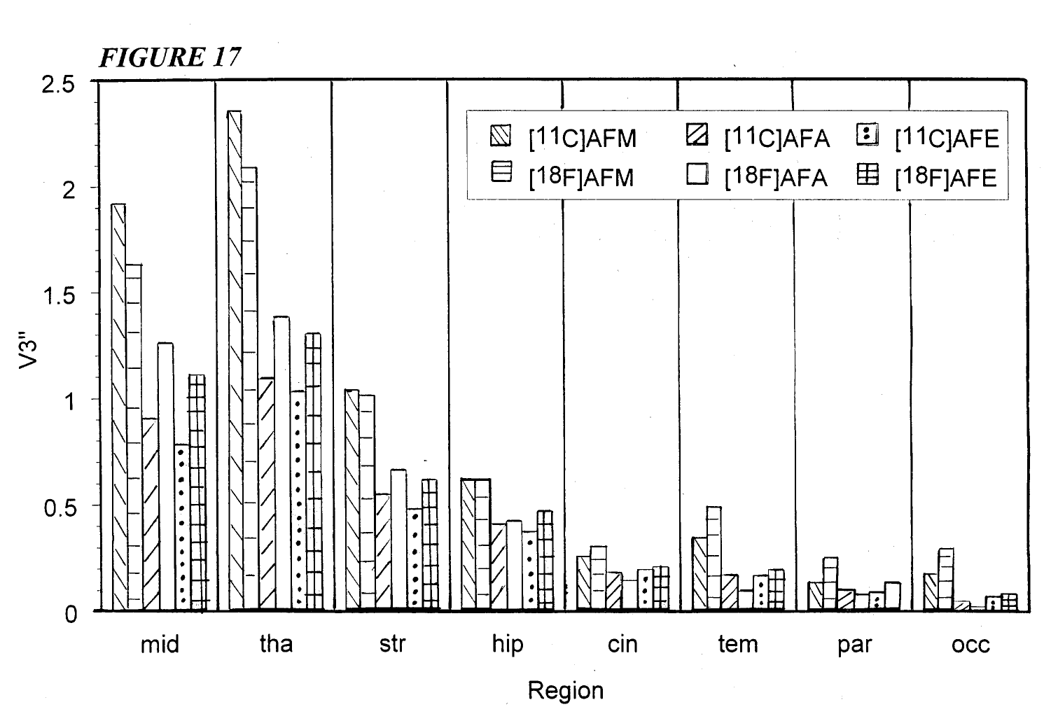

Activity distribution: Over time, activity concentrated in brain regions with high SERT densities, i.e. midbrain, thalamus, and striatum. Intermediate levels were found in the hippocampus, temporal and cingulate cortices, and lower values were found in other cortical regions. Lowest values were observed in the cerebellum (FIG. 4). With all tracers, activity accumulation was readily noticeable in the central structures associated with high SERT densities (midbrain, thalamus and striatum). However, the higher density of SERT in medial temporal lobe structures such as hippocampus compared to the neocortex translated into a visually detectable activity contrast only in the [¹¹C]DASB and [¹¹C]AFM images.

FIGS. 5-9 present time-activity curves for each ligand in plasma (corrected for metabolites) and in brain, where a subset of representative regions (thalamus, striatum, temporal cortex and cerebellum) are presented. To facilitate comparison between ligands, experiments that are presented were acquired in the same animal (baboon A), activities were normalized to the injected doses, and the axis scales were kept constant. It can be appreciated that [¹¹C]McN 5652 (FIG. 5B) and [¹¹C]ADAM (FIG. 6B) displayed a similar pattern of brain kinetics: the uptake was relatively slow and protracted (peak uptake is smooth), the wash-out was slow, and only modest differences were observed between the regions. In contrast, [¹¹C]DASB (FIG. 7B) presented an initial rapid uptake, an early and sharp peak, a rapid wash-out, and larger differences between regions. [¹¹C]DAPA kinetic profile (FIG. 8B) was similar to [¹¹C]McN 5652 and [¹¹C]ADAM, in that the uptake showed a smooth and protracted peak, but differed from these two tracers in that the differences in activity between regions were larger. [¹¹C]AFM (FIG. 9B) shared with [¹¹C]DASB a sharp peak and faster wash-out, but the peak occurred later and the differences in activity between regions were more accentuated.

Regional uptake: Regional peak uptake times are presented in Table 6. Variability in regional peak uptake times was evaluated with repeated measure ANOVA, with regions as repeated measure and baboons and ligands as cofactors. Significant differences in peak uptake were observed between regions (p<0.0001), between tracers (p=0.008) and, at trend level, between baboons (p=0.07). Regional rank order for peak uptake time was cerebellum<occipital cortex≈cingulate cortex<temporal cortex<striatum≈hippocampus<thalamus≈midbrain. Rank order of mean regional peak uptake time for radioligands was [¹¹C]DASB (19±9 minutes)<[¹¹C]McN 5652 (28±12 minutes)<[¹¹C]ADAM (37±18 minutes)≈[¹¹C]AFM (38±19 minutes)<[¹¹C]DAPA (50±20 minutes). Post-hoc analysis revealed that [¹¹]DASB peaked significantly earlier than [¹¹C]ADAM, [¹¹C]AFM, and [¹¹C]DAPA (p<0.05). Baboon A showed earlier peak uptake time than baboon B (p<0.05).

TABLE 6
Regional uptake peak times (minutes)

| | | | | | | Temporal | Cingulate | Occipital |
|---|---|---|---|---|---|---|---|---|
| Radiotracer | Cerebellum | Midbrain | Thalamus | Striatum | Hippocampus | Cortex | Cortex | Cortex |
| ¹¹C]McN 5652 | 15 ± 3 | 39 ± 13 | 41 ± 6 | 34 ± 10 | 36 ± 8 | 27 ± 7 | 17 ± 3 | 17 ± 3 |
| [¹¹C]ADAM | 14 ± 5 | 49 ± 22 | 51 ± 24 | 39 ± 15 | 46 ± 8 | 39 ± 13 | 32 ± 17 | 30 ± 18 |
| [¹¹C]DASB | 12 ± 5 | 27 ± 13 | 24 ± 10 | 22 ± 10 | 22 ± 10 | 19 ± 5 | 14 ± 3 | 16 ± 4 |
| [¹¹C]DAPA | 24 ± 10 | 71 ± 13 | 69 ± 13 | 51 ± 19 | 64 ± 15 | 49 ± 10 | 36 ± 8 | 36 ± 8 |
| [¹¹C]AFM | 23 ± 11 | 56 ± 24 | 54 ± 22 | 44 ± 21 | 39 ± 16 | 34 ± 14 | 28 ± 11 | 30 ± 14 |

Values are mean ± SD of four experiments.

Following the peak, ligands showed differences in the rate of wash-out Table 7. Wash-out was estimated by calculating the relative decrease in regional activity captured during the time-frame of the scan (relative decrease from peak time to last frame). Variability in decrease in regional activity was evaluated with repeated measure ANOVA, with regions as repeated measure and baboons and ligands as cofactors. Significant differences in activity wash-out were observed between regions (p<0.0001), between tracers (p=0.002) and, at trend level, between baboons (p=0.08). Rank order for mean regional wash-out was [¹¹C]DASB (52±19% decrease between peak time and last scan)>[¹¹C]AFM (32±26%)>[¹¹C]ADAM (25±14%) [¹¹C]McN 5652 (24±13%)>[¹¹C]DAPA (12±11%). Post-hoc analysis showed that [¹¹C]DASB washed out significantly faster than all other ligands, and that [¹¹C]AFM washed-out significantly faster than [¹¹C]DAPA. Baboon A showed significantly more wash-out than baboon B during the time frame of the scan.

TABLE 7
Regional activity wash-out: % decrease from peak to last scan (90 minutes)

| | | | | | | Temporal | Cingulate | Occipital |
|---|---|---|---|---|---|---|---|---|
| Radiotracer | Cerebellum | Midbrain | Thalamus | Striatum | Hippocampus | Cortex | Cortex | Cortex |
| [¹¹C]McN 5652 | 44 ± 6% | 8 ± 5% | 12 ± 4% | 18 ± 7% | 16 ± 3% | 24 ± 6% | 34 ± 3% | 34 ± 3% |
| [¹¹C]ADAM | 45 ± 8% | 10 ± 7% | 12 ± 5% | 23 ± 6% | 15 ± 6% | 25 ± 8% | 34 ± 7% | 36 ± 10% |
| [¹¹C]DASB | 68 ± 14% | 32 ± 19% | 36 ± 16% | 49 ± 18% | 46 ± 19% | 58 ± 13% | 64 ± 10% | 65 ± 11% |
| [¹¹C]DAPA | 29 ± 14% | 2 ± 3% | 3 ± 4% | 8 ± 8% | 4 ± 5% | 11 ± 5% | 19 ± 9% | 18 ± 7% |
| [¹¹C]AFM | 48 ± 28% | 16 ± 22% | 18 ± 25% | 29 ± 31% | 26 ± 27% | 36 ± 29% | 43 ± 27% | 42 ± 27% |

Values are mean ± SD of four experiments.

Regional activity ratios: ROI to cerebellum activity ratios measured at 90 minutes are presented in Table 8. Variability in these ratios was evaluated with repeated measure ANOVA, with regions as repeated measure and baboons and ligands as cofactors. Significant differences in regional ratios were observed between regions (p<0.0001), between tracers (p=0.0001) and between baboons (p=0.01) (FIG. 10). Highest ratios were obtained with [¹¹C] AFM and [¹¹C]DASB. Ratios measured with these two ligands were not significantly different, but were significantly higher than ratios obtained with [¹¹C]ADAM, [¹¹C]DAPA and [¹¹C]McN 5652. Ratios measured with [¹¹C]ADAM, [¹¹C]DAPA, and [¹¹C]McN 5652 were not significantly different from each other. Baboon A showed significantly higher ratios compared to baboon B (p<0.05).

TABLE 8
Region of interest to cerebellum activity ratios at 90 minutes

| | | | | | Temporal | Cingulate | Occipital |
|---|---|---|---|---|---|---|---|
| Radiotracer | Midbrain | Thalamus | Striatum | Hippocampus | Cortex | Cortex | Cortex |
| [¹¹C]McN 5652 | 2.02 ± 0.10 | 2.11 ± 0.09 | 1.73 ± 0.09 | 1.40 ± 0.02 | 1.28 ± 0.08 | 1.23 ± 0.04 | 1.15 ± 0.11 |
| [¹¹C]ADAM | 1.96 ± 0.18 | 2.05 ± 0.23 | 1.65 ± 0.13 | 1.45 ± 0.06 | 1.33 ± 0.08 | 1.29 ± 0.07 | 1.14 ± 0.09 |
| [¹¹C]DASB | 3.33 ± 0.13 | 3.36 ± 0.04 | 2.16 ± 0.10 | 1.81 ± 0.12 | 1.39 ± 0.21 | 1.27 ± 0.16 | 1.10 ± 0.12 |
| [¹¹C]DAPA | 2.05 ± 0.31 | 2.15 ± 0.30 | 1.69 ± 0.12 | 1.42 ± 0.13 | 1.35 ± 0.16 | 1.27 ± 0.05 | 1.19 ± 0.08 |
| [¹¹C]AFM | 3.48 ± 1.09 | 3.51 ± 0.72 | 2.10 ± 0.24 | 1.84 ± 0.40 | 1.44 ± 0.20 | 1.30 ± 0.09 | 1.26 ± 0.18 |

Values are mean ± SD of four experiments.

Kinetic rate constants: Values of K₁ and k₂ are presented in Table 9. Variability was evaluated with repeated measure ANOVA, with regions as repeated measures and baboons and ligands as cofactors. Significant differences in K₁ were observed between regions (p<0.0001), between tracers (p=0.055) and, at trend level, between baboons (p=0.10). [¹¹C]AFM had the highest K₁ (average of all regions and all animals of 1.44±0.40 mL g⁻¹ min⁻¹), followed by [¹¹C]DASB (1.21±0.22 mL g⁻¹ min¹), [¹¹C]ADAM (1.09±0.35 mL g⁻¹ min⁻¹), [¹¹C]McN 5652 (0.93±0.18 mL g⁻¹ min⁻¹) and [¹¹C]DAPA (0.85±0.23 mL g min). Post-hoc analysis showed that [¹¹C]AFM K₁ was significantly higher than [¹¹C]McN 5652 K₁ and [¹¹C]DAPA K₁.

Significant differences in k₂ were observed between regions (p<0.0001), between tracers (p=0.0013) but not between baboons (p=0.23). [¹¹C]DASB had the highest k₂ (average of all regions and all animals of 0.047±0.018 min⁻¹), followed by [¹¹C] AFM (0.030±0.015 min⁻¹), [¹¹C]ADAM (0.028±0.011 min⁻¹), [¹¹C]McN 5652 (0.025±0.008 min⁻¹) and [¹¹C]DAPA (0.020±0.008 min⁻¹). Post-hoc analysis showed that [¹¹C]DASB k₂ was significantly higher than the four other ligands, and that [¹¹C]AFM k₂ was higher than [¹¹C]DAPA k₂.

TABLE 9
Kinetic analysis: rate constants and total distribution volumes

| | | | | | | | | |
|---|---|---|---|---|---|---|---|---|
| | | | | | | Temporal | Cingulate | Occipital |
| Radiotracer | Cerebellum | Midbrain | Thalamus | Striatum | Hippocampus | Cortex | Cortex | Cortex |

K₁ (mL g⁻¹ min⁻¹)

| | | | | | | | | |
|---|---|---|---|---|---|---|---|---|
| [¹¹C]McN | 1.05 ± 0.20 | 0.92 ± 0.12 | 1.03 ± 0.15 | 0.95 ± 0.12 | 0.74 ± 0.12 | 0.83 ± 0.19 | 1.01 ± 0.24 | 0.95 ± 0.18 |
| 5652 | | | | | | | | |
| [¹¹C]ADAM | 1.23 ± 0.44 | 1.00 ± 0.35 | 1.14 ± 0.32 | 1.15 ± 0.45 | 0.85 ± 0.32 | 1.02 ± 0.37 | 1.20 ± 0.38 | 1.12 ± 0.33 |
| [¹¹C]DASB | 1.36 ± 0.24 | 1.23 ± 0.20 | 1.35 ± 0.29 | 1.27 ± 0.21 | 0.98 ± 0.20 | 1.05 ± 0.09 | 1.26 ± 0.09 | 1.15 ± 0.24 |
| [¹¹C]DAPA | 0.99 ± 0.29 | 0.86 ± 0.27 | 0.89 ± 0.26 | 0.90 ± 0.26 | 0.67 ± 0.16 | 0.78 ± 0.18 | 0.91 ± 0.24 | 0.83 ± 0.17 |
| [¹¹C]AFM | 1.52 ± 0.49 | 1.57 ± 0.41 | 1.68 ± 0.42 | 1.48 ± 0.48 | 1.15 ± 0.35 | 1.25 ± 0.34 | 1.48 ± 0.40 | 1.35 ± 0.37 |

K₂ (min⁻¹)

| | | | | | | | | |
|---|---|---|---|---|---|---|---|---|
| [¹¹C]McN | 0.038 ± 0.007 | 0.017 ± 0.003 | 0.019 ± 0.003 | 0.021 ± 0.003 | 0.020 ± 0.003 | 0.025 ± 0.006 | 0.031 ± 0.007 | 0.031 ± 0.006 |
| 5652 | | | | | | | | |
| [¹¹C]ADAM | 0.043 ± 0.012 | 0.018 ± 0.006 | 0.020 ± 0.006 | 0.025 ± 0.008 | 0.021 ± 0.006 | 0.027 ± 0.009 | 0.033 ± 0.009 | 0.034 ± 0.010 |
| [¹¹C]DASB | 0.078 ± 0.013 | 0.027 ± 0.005 | 0.028 ± 0.006 | 0.040 ± 0.007 | 0.036 ± 0.006 | 0.047 ± 0.004 | 0.060 ± 0.004 | 0.059 ± 0.011 |
| [¹¹C]DAPA | 0.034 ± 0.009 | 0.012 ± 0.003 | 0.012 ± 0.004 | 0.017 ± 0.005 | 0.015 ± 0.003 | 0.019 ± 0.004 | 0.025 ± 0.006 | 0.024 ± 0.005 |
| [¹¹C]AFM | 0.051 ± 0.021 | 0.017 ± 0.006 | 0.017 ± 0.006 | 0.025 ± 0.010 | 0.023 ± 0.008 | 0.030 ± 0.010 | 0.039 ± 0.012 | 0.036 ± 0.011 |

V_{T} (mL g⁻¹)

| | | | | | | | | |
|---|---|---|---|---|---|---|---|---|
| [¹¹C]McN | 27.7 ± 4.0 | 53.6 ± 9.3 | 56.5 ± 10.3 | 45.3 ± 7.0 | 37.2 ± 5.9 | 33.9 ± 4.2 | 33.1 ± 4.7 | 30.6 ± 3.9 |
| 5652 | | | | | | | | |
| [¹¹C]ADAM | 28.5 ± 4.8 | 57.0 ± 10.2 | 59.9 ± 10.1 | 46.3 ± 8.4 | 41.1 ± 8.0 | 38.6 ± 9.7 | 37.3 ± 8.5 | 33.5 ± 8.3 |
| [¹¹C]DASB | 17.3 ± 0.5 | 46.4 ± 4.3 | 47.5 ± 5.0 | 31.7 ± 1.7 | 27.3 ± 1.4 | 22.5 ± 2.1 | 21.1 ± 0.9 | 19.3 ± 1.5 |
| [¹¹C]DAPA | 29.3 ± 2.9 | 69.2 ± 7.0 | 74.4 ± 3.1 | 52.1 ± 3.0 | 44.7 ± 3.4 | 40.5 ± 6.0 | 37.1 ± 3.8 | 35.0 ± 4.8 |
| [¹¹C]AFM | 30.6 ± 3.9 | 96.3 ± 15.3 | 101.9 ± 20.4 | 61.9 ± 10.4 | 51.5 ± 6.1 | 42.4 ± 4.5 | 39.0 ± 4.7 | 37.9 ± 3.6 |

Values are mean ± SD of four experiments.

Total Regional Distribution Volumes: Values of V_{T} are presented in Table 9. Significant differences in V_{T} were observed between regions (p<0.0001), between tracers (p=0.0002) but not between baboons (p=0.91). [¹¹C]AFM showed the highest V_{T} (average of all regions and all animals, 57.5±27.5 mL g⁻¹), followed by [¹¹C]DAPA (47.8±16 mL g⁻¹), [¹¹C]ADAM (42.8±12.9 mL g⁻¹), [¹¹C]McN 5652 (39.7±11.8 mL g⁻¹) and [¹¹C]DASB (29.2±11.5 mL g⁻¹).

Minimal scanning time to reach time-invariance criteria: Table 10 lists, by tracer and by region, the minimal scanning time required to reach time invariance criteria in the derivation of V_{T}. Significant differences between tracers were observed (p<0.001). Average scanning time required for [¹¹C]DAPA (mean of all regions, 68±9 minutes) and [¹¹C]AFM (62±16 minutes) were similar to [¹¹C]McN 5652 (62±5 minutes), while [¹¹C]ADAM (44±13 minutes) and [¹¹C]DASB (36±11 minutes) minimal scanning time were significantly shorter compared to the time required for the other three ligands.

TABLE 10
Minimal scanning time (minutes) to reach time-independent measurement for VT.

| | | | | | | Temporal | Cingulate | Occipital | Average |
|---|---|---|---|---|---|---|---|---|---|
| Radiotracer | Cerebellum | Midbrain | Thalamus | Striatum | Hippocampus | Cortex | Cortex | Cortex | by tracer |
| [¹¹C]McN 5652 | 60 | 70 | 60 | 60 | 70 | 70 | 60 | 60 | 64 ± 5 |
| [¹¹C]ADAM | 40 | 70 | 50 | 40 | 50 | 30 | 30 | 40 | 44 ± 13 |
| [¹¹C]DASB | 30 | 60 | 40 | 30 | 30 | 30 | 30 | 40 | 36 ± 11 |
| [¹¹C]DAPA | 60 | 80 | 70 | 70 | 80 | 60 | 60 | 60 | 68 ± 9 |
| [¹¹C]AFM | 30 | 70 | 70 | 70 | 80 | 70 | 50 | 60 | 63 ± 16 |
| Average by region | 44 ± 15 | 70 ± 7 | 58 ± 13 | 54 ± 18 | 62 ± 22 | 52 ± 20 | 46 ± 15 | 52 ± 11 | 55 ± 15 |

Minimal scanning time was derived from the analysis of four experiments.
See text for criteria of time-invariance.

FIG. 11 displays the bias associated with shorter scanning time (40 to 90 minutes): [¹¹C]McN 5652 and [¹¹C]AFM showed an overestimation of V_{T} with shorter scanning times, [¹¹C]DAPA showed an underestimation, and only minimal biases were associated with scanning time above 40 minutes for [¹¹C]DASB and [¹¹C]ADAM.

Nonspecific distribution volumes: Values of cerebellar V_{T} are presented in Table 9. Significant differences in cerebellar V_{T} were observed between tracers (p=0.0005) but not between baboons (p=0.69). [¹¹C]DASB showed significantly lower cerebellar V_{T} compared to the four other ligands, which did not significantly differ from one another. Because reliable measures of f₁ were available only for [¹¹C]DASB and [¹¹C]AFM, the free fraction in the nonspecific distribution volume, f₂, was calculated only for these two tracers. [¹¹C]DASB f₂ was 0.77±0.08%, significantly higher than [¹¹C]AFM f₂ (0.32±0.04%).

Binding potential: Significant differences in BP (Table 11) were observed between regions (p<0.0001), between tracers (p<0.0001) but not between baboons (p=0.83). Rank order of BP values was [¹¹C]AFM>[¹¹C]DAPA>[¹¹C]ADAM>[¹¹C]DASB≈[¹¹C]McN 5652. [¹¹C]AFM BP was significantly higher compared to the BP of the four other tracers, and [¹¹C]DAPA BP was significantly higher than [¹¹C]DASB BP and [¹¹C]McN 5652 BP.

TABLE 11
Binding potential (mL g⁻¹).

| | | | | | Temporal | Cingulate | Occipital |
|---|---|---|---|---|---|---|---|
| Radiotracer | Midbrain | Thalamus | Striatum | Hippocampus | Cortex | Cortex | Cortex |
| [¹¹C]McN 5652 | 25.9 ± 6.1 | 28.8 ± 6.9 | 17.6 ± 4.0 | 9.5 ± 2.3 | 6.2 ± 1.8 | 5.4 ± 0.8 | 2.9 ± 1.6 |
| [¹¹C]ADAM | 28.5 ± 6.7 | 31.4 ± 6.1 | 17.8 ± 3.7 | 12.6 ± 3.3 | 10.1 ± 5.0 | 8.8 ± 3.9 | 4.9 ± 3.8 |
| [¹¹C]DASB | 29.1 ± 4.0 | 30.2 ± 4.9 | 14.4 ± 1.5 | 10.0 ± 1.3 | 5.1 ± 1.9 | 3.8 ± 0.8 | 2.0 ± 1.1 |
| [¹¹C]DAPA | 40.0 ± 4.3 | 45.1 ± 1.8 | 22.9 ± 0.9 | 15.5 ± 0.7 | 11.2 ± 3.3 | 7.9 ± 1.0 | 5.7 ± 1.9 |
| [¹¹C]AFM | 65.8 ± 11.7 | 71.3 ± 16.5 | 31.3 ± 6.5 | 20.9 ± 2.5 | 11.8 ± 0.9 | 8.5 ± 1.0 | 7.3 ± 1.3 |

Values are mean ± SD of four experiments.

The Bₘₐₓ/K_{D} ratios could be calculated for [¹¹C]AFM and [¹¹C]DASB, but not for the other three ligands, given that their free fraction could not be measured. For example, in the thalamus, [¹¹C]AFM and [¹¹C]DASB Bₘₐₓ/K_{D} values were 742±168 mL g⁻¹ and 225±30 mL g⁻¹, respectively. Assuming that these tracers bind to the same number of binding site, these data indicate that the in vivo affinity of [¹¹C]AFM is 3.3 times higher than that of [¹¹C]DASB.

Specific to nonspecific partition coefficient: Table 12 lists values of V₃″ measured in each experiment. Significant differences in V₃″ were observed between regions (p<0.0001), between tracers (p<0.0001) but not between baboons (p=0.75). Highest V₃″ values were observed in the thalamus and midbrain, followed by striatum, hippocampus, temporal cortex, cingulate cortex and occipital cortex. Radioligand rank order of V₃″ values was [¹¹C]AFM>[¹¹C]DASB≈[¹¹C]DAPA>[¹¹C]ADAM≈[¹¹C]McN 5652. The differences in V₃″ between [¹¹C]AFM and the four other tracers were significant. [¹¹C]DASB and [¹¹C]DAPA V₃″ were not significantly different from each other, but significantly higher than [¹¹C]ADAM and [¹¹C]McN 5652 V₃″. [¹¹C]ADAM and [¹¹C]McN 5652 V₃″ were not significantly different FIG. 12).

TABLE 12
Equilibrium specific to non-specific partition coefficient (V3″).

| | | | | | | Temporal | Cingulate | Occipital |
|---|---|---|---|---|---|---|---|---|
| Radiotracer | Baboon | Midbrain | Thalamus | Striatum | Hippocampus | Cortex | Cortex | Cortex |
| [¹¹C]McN 5652 | A | 1.16 | 1.17 | 0.81 | 0.41 | 0.34 | 0.21 | 0.13 |
| | A | 0.78 | 0.82 | 0.49 | 0.29 | 0.23 | 0.17 | 0.18 |
| | B | 1.00 | 1.15 | 0.66 | 0.38 | 0.18 | 0.20 | 0.05 |
| | B | 0.80 | 1.00 | 0.60 | 0.30 | 0.16 | 0.20 | 0.07 |
| Mean ± SD | | 0.94 ± 0.18 | 1.04 ± 0.16 | 0.64 ± 0.13 | 0.34 ± 0.06 | 0.23 ± 0.08 | 0.19 ± 0.02 | 0.11 ± 0.06 |
| [¹¹C]ADAM | A | 1.04 | 1.18 | 0.65 | 0.41 | 0.41 | 0.28 | 0.15 |
| | A | 0.81 | 0.92 | 0.59 | 0.49 | 0.47 | 0.43 | 0.31 |
| | B | 0.91 | 1.05 | 0.56 | 0.38 | 0.18 | 0.23 | 0.07 |
| | B | 1.25 | 1.26 | 0.68 | 0.47 | 0.30 | 0.26 | 0.12 |
| Mean ± SD | | 1.00 ± 0.19 | 1.10 ± 0.15 | 0.62 ± 0.06 | 0.44 ± 0.05 | 0.34 ± 0.13 | 0.30 ± 0.09 | 0.16 ± 0.11 |
| [¹¹C]DASB | A | 1.46 | 1.42 | 0.72 | 0.48 | 0.34 | 0.26 | 0.15 |
| | A | 1.66 | 1.68 | 0.89 | 0.65 | 0.42 | 0.25 | 0.18 |
| | B | 1.64 | 1.79 | 0.83 | 0.62 | 0.21 | 0.21 | 0.04 |
| | B | 1.96 | 2.07 | 0.87 | 0.54 | 0.21 | 0.16 | 0.10 |
| Mean ± SD | | 1.68 ± 0.21 | 1.74 ± 0.27 | 0.83 ± 0.08 | 0.58 ± 0.08 | 0.29 ± 0.10 | 0.22 ± 0.04 | 0.11 ± 0.06 |
| [¹¹C]DAPA | A | 1.41 | 1.49 | 0.73 | 0.54 | 0.48 | 0.29 | 0.22 |
| | A | 1.37 | 1.40 | 0.72 | 0.48 | 0.42 | 0.26 | 0.25 |
| | B | 1.42 | 1.81 | 0.91 | 0.56 | 0.30 | 0.25 | 0.15 |
| | B | 1.27 | 1.52 | 0.79 | 0.54 | 0.32 | 0.27 | 0.15 |
| Mean ± SD | | 1.37 ± 0.07 | 1.55 ± 0.18 | 0.79 ± 0.09 | 0.53 ± 0.04 | 0.38 ± 0.09 | 0.27 ± 0.02 | 0.19 ± 0.05 |
| [¹¹C]AFM | A | 2.13 | 2.10 | 0.97 | 0.70 | 0.38 | 0.30 | 0.29 |
| | A | 2.18 | 2.10 | 0.92 | 0.75 | 0.46 | 0.26 | 0.30 |
| | B | 1.94 | 2.46 | 1.06 | 0.61 | 0.35 | 0.30 | 0.18 |
| | B | 2.33 | 2.58 | 1.11 | 0.69 | 0.37 | 0.25 | 0.20 |
| Mean ± SD | | 2.14 ± 0.16 | 2.31 ± 0.25 | 1.01 ± 0.08 | 0.69 ± 0.06 | 0.39 ± 0.05 | 0.28 ± 0.02 | 0.24 ± 0.06 |

Relative distribution of binding sites: V₃″ corresponds to f₂Bmax/K_{D} ratio. As f₂ and K_{D} were not expected to vary between regions, regional variability in V₃″ should reflect regional variability in Bₘₐₓ. Under the hypothesis that each tracer binds to the same population of binding sites (i.e. SERT), the relative regional distribution of V₃″ should be similar for each tracer. This prediction was tested by expressing, for each experiment, regional V₃″ in percentage of thalamus V₃″. Combining all tracers and all baboons, midbrain V₃″ was equal to 92±8% of thalamic V₃″. Striatal V₃″ values were 52±7% of thalamic V₃″ and hippocampal V₃″ was 34±6%. In the neocortical regions, these values were 23±9%, 18±8% and 12±7% for temporal, cingulate and occipital cortices, respectively. Variability between relative regional V₃″ was assessed with repeated measure ANOVA, with regions as repeated measure and ligand as covariate. Significant differences were observed between regions (p<0.001), but not between tracers (p=0.20), and a significant tracer by region interaction was observed (p=0.002). The interaction plot is presented in FIG. 13. To identify the source of this interaction, all regions were analyzed separately. No between-ligand difference in relative V₃″ were observed in the midbrain (p=0.67), hippocampus (p=0.15), temporal cortex (p=0.18) and occipital cortex (p=0.75). In the striatum, [¹¹C]McN 5652 and [¹¹C]ADAM displayed a significantly larger relative V₃″ value compared to [¹¹C]DASB, [¹¹C]DAPA, and [¹¹C]AFM. In the cingulate, [¹¹C]ADAM displayed significantly larger relative V₃″ value compared to the four other tracers. In fact, this relative excess of [¹¹C]ADAM V₃″ was also observed in the temporal and cingulate cortex, although these differences did not reach significance in these regions.

Reproducibility of regional SERT V₃″ measurement: The test/retest reproducibility, calculated as the absolute value of the difference between test 1 and test 2 divided by their average value is presented in Table 13. Significant differences in reproducibility were observed between regions (p=0.04), and, at trend level, between ligands (p=0.062). Reproducibility was relatively uniform across regions, with the exception of the occipital cortex, which showed significantly worse reproducibility of V₃″ measurement compared to all other regions. The rank order of reproducibility for ligands was, from the best to the worst, [¹¹C]AFM≈[¹¹C]DAPA>[¹¹C]DASB>[¹¹C]McN 5652>[¹¹C]ADAM. The improved reproducibility of [¹¹C]AFM and [¹¹C]DAPA compared to [¹¹C]McN 5652 or [¹¹C]ADAM was significant.

TABLE 13
Reproducibility of V3″ measurement.

| | | | | | Temporal | Cingulate | Occipital | Average by |
|---|---|---|---|---|---|---|---|---|
| Radiotracer | Midbrain | Thalamus | Striatum | Hippocampus | Cortex | Cortex | Cortex | tracer |
| [¹¹C]McN 5652 | 31 ± 11% | 25 ± 15% | 30 ± 28% | 28 ± 8% | 25 ± 19% | 10 ± 13% | 35 ± 1% | 26 ± 8% |
| [¹¹C]ADAM | 28 ± 4% | 21 ± 5% | 15 ± 7% | 20 ± 3% | 32 ± 25% | 28 ± 19% | 63 ± 10% | 30 ± 16% |
| [¹¹C]DASB | 15 ± 4% | 15 ± 1% | 13 ± 12% | 22 ± 12% | 10 ± 14% | 14 ± 15% | 49 ± 43% | 20 ± 13% |
| [¹¹C]DAPA | 7 ± 6% | 12 ± 8% | 7 ± 9% | 8 ± 6% | 10 ± 7% | 9 ± 1% | 7 ± 6% | 9 ± 2% |
| [¹¹C]AFM | 10 ± 11% | 3 ± 3% | 5 ± 0% | 9 ± 5% | 11 ± 10% | 14 ± 4% | 9 ± 5% | 9 ± 4% |
| Average by region | 18 ± 10% | 15 ± 9% | 14 ± 10% | 18 ± 9% | 18 ± 10% | 15 ± 8% | 32 ± 24% | 19 ± 6% |

Values are mean ± SD of two test/retest pairs.

### C. Discussion

In this study, the imaging qualities of four new PET radioligands for the SERT, [¹¹C]ADAM, [¹¹C] DASB, [¹¹C]DAPA and [¹¹C]ADAM, were compared in baboons to the current reference tracer, [¹¹C]McN 5652. The results indicate that both [¹¹C]DASB and [¹¹C]AFM provide substantial improvement over [¹¹C]McN 5652 for SERT imaging.

The introduction of [¹¹C]McN 5652 in the mid-nineties represented an important advance, as this radioligand was the first imaging agent successfully used to image SERT in baboons (Szabo et al., 1995a) and humans (Szabo et al., 1995b) in vivo with PET. The significance of this achievement must be appreciated in the context of years of failed attempts to develop a suitable radioligand for SERT (see introduction). Prior to [¹¹C]McN 5652, only [¹²³I]β-CIT was available for in vivo visualization of SERT in the human brain, and the use of this ligand for SERT imaging was restricted to the midbrain region (Laruelle et al., 1993b). [¹¹C]McN 5652, being more selective than [¹²³I]β-CIT, was suitable to image SERT, not only in the midbrain, but also in the thalamus and striatum. The modest selectivity of [¹¹C]McN 5652 relative to norepinephrine transporters (Shank et al., 1988) did not appear to create a significant problem in vivo, at least in mice, where 5 mg/kg desipramine failed to affect specific binding (Suchiro et al., 1993b).

However, [¹¹C]McN 5652 provides a relatively low signal to noise ratio, thus precluding the reliable quantification of SERT in regions of the limbic system, where the concentrations of SERT is lower than those in the midbrain, striatum and thalamus. In addition, the uptake of [¹¹C]McN 5652 was relatively protracted in human. As a result, at least 120 minutes of data acquisition was required to obtain time-independent estimates of V_{T} in regions of high SERT density, such as the midbrain. And finally, the free fraction of [¹¹C]McN 5652 in plasma is too low to be measured accurately using the standard ultracentrifugation technique. Thus, while [¹¹C]McN 5652 was suitable to image SERT in humans, the limitations of this ligand were also recognized (Szabo et al., 1999; Buck et al., 2000; Parsey et al., 2000).

In 1999, Kung and colleagues reported the synthesis and characterization of a new radioiodinated compound, [¹²³I]IDAM (K_{D} for SERT of 0.25 nM), based on a new class of selective serotonin reuptake inhibitors (Acton et al., 1999a; Kung et al., 1999; Oya et al., 1999). [¹²³I]IDAM was the first selective radioligand to image SERT with SPECT. In the same year, this group also reported [¹²³I]ODAM, a phenoxy derivative of [¹²³I]IDAM, that was more resistant to metabolism but had a slightly lower affinity for SERT, and lower midbrain to cerebellum ratio (Acton et al., 1999b; Zhuang et al., 2000). In 2000, Kung and colleagues reported on a third ligand, [¹²³I]ADAM, an amino derivative of [¹²³I]IDAM (Choi et al., 2000; Oya et al., 2000; Acton et al., 2001). In vitro Kᵢ of [¹²⁵I]ADAM in rat brain membranes (0.15 nM) was comparable to [¹²⁵I]IDAM (0.25 nM). However, [¹²³I]ADAM displayed four times higher target to background ratio in vivo compared to [¹²³I]IDAM, thus making it a radioligand of choice to image SERT with SPECT. In 2001, Vercouille et al. (2001) reported the labeling of ADAM with C-11. However, the properties of [¹¹C]ADAM as a PET imaging agent have not yet been reported.

At about the same time, Wilson and colleagues synthesized and evaluated a number of compounds in this same series as C-11 labeled candidate PET radioligands (Wilson et al., 1999; and Wilson et al., 2000). Among these [¹¹C]DASB (K_{D}=1.10 nM) emerged as the most promising agent, based on the fast wash-out kinetic in rats. Further, two of these new compounds, [¹¹C]DASP and [¹¹C]DAPP, were evaluated in humans (Houle et al., 2000). In humans, [¹¹C]DASB displayed higher brain uptake than [¹¹C]DAPP. Additional characterization of [¹¹C]DASB in humans revealed that its binding was inhibited following pretreatment with paroxetine and citalopram (Houle et al., 2000). Kinetic analysis of [¹¹C]DASB uptake in humans demonstrated that a one-tissue compartment model was appropriate to derive [¹¹C]DASB distribution volumes (Ginovart et al., 2001).

Several other derivatives in this phenylamine series of compounds have been prepared and characterized (Huang et al. 2001a; Huang et al. 2001b). [¹¹C]DAPA, the bromo analog of ADAM, showed promising features in rodent studies (Huang et al., 2001b). Two fluorinated analogues were also prepared and evaluated, [¹¹C]AFA and [¹¹C]AFM, both suitable for potential labeling with F-18 (Huang et al., 2001a). [¹¹C]AFM displayed superior in vivo binding properties in rodents and baboons compared to [¹¹C]AFA, and was selected for further development.

Consequently, over the last three years, the portfolio of available SERT imaging agents was dramatically changed. Whereas, until recently, only one selective tracer was available, there are now several highly selective and promising candidate PET radioligands. Due to differences in methodologies between laboratories and the limited nature of outcome measures used in the initial evaluation of tracers (tissue to cerebellum ratio at one time-point), it was difficult to compare the potential of these new radioligands based on published literature. Therefore, this study was designed as a collaborative effort to evaluate the imaging qualities of these tracers under identical experimental conditions. This evaluation included in vitro measurement of affinity and lipophilicity, and in vivo PET imaging experiments in two baboons.

For each parameter, a desired property was a priori defined to provide improvement over [¹¹C]McN 5652. Among these, the most critical elements included: 1) higher V₃″, to provide better signal to noise contrast for the quantification of SERT in regions of relatively low SERT density, such as regions of the limbic system; 2) reduced scanning time required to derive time-independent estimate of VT, to improve subject compliance and reduce noise associated with C-11 decay; 3) increased plasma free fraction, to be able to control for this parameter in clinical studies. Note that properties 1 and 2 are essentially conflicting: everything else being equal, a higher in vivo affinity will result in higher V₃″ values but in longer time required to reach equilibrium.

Table 14 summarizes the rank order of the tested compounds for each parameter. The symbol < and < denote statistically significant differences, and denote differences that are not statistically significant.

TABLE 14
Comparative evaluation of tested radiotracers.

| | | Parameter | Desired property ^{a} | Comparative evaluation ^{b} |
|---|---|---|---|---|
| In vitro | | Affinity | High | [¹¹C]AFM ≈ [¹¹C]ADAM ≈ [¹¹C]McN 5652 ≈ [¹¹C]DAPA > [¹¹C]DASB |
| | | Lipophilicity | Low | [¹¹C]DASB ≈ [¹¹C]AFM < [¹¹C]McN 5652 < [¹¹C]DAPA < [¹¹C]ADAM |
| In vivo | Plasma | Metabolism | Fast | [¹¹C]AFM > [¹¹C]McN 5652 ≈ [¹¹C]DASB ≈ [¹¹C]ADAM ≈ [¹¹C]DAPA |
| | | Clearance | Fast | [¹¹C]AFM ≈ [¹¹C]DASB > [¹¹C]ADAM ≈ [¹¹C]McN 5652 ≈ [¹¹C]DAPA |
| | | Free fraction | High | [¹¹C]DASB > [¹¹C]AFM > [¹¹C]DAPA ≈ [¹¹C]ADAM ≈ [¹¹C]McN 5652 |
| | Brain | Peak uptake | Early | [¹¹C]DASB > [¹¹C]McN 5652 ≈ [¹¹C]ADAM ≈ [¹¹C]AFM ≈ [¹¹C]DAPA |
| | | Wash-out | Early | [¹¹C]DASB > [¹¹C]AFM > [¹¹C]ADAM ≈ [¹¹C]McN 5652 ≈ [¹¹C]DAPA |
| | | Ratios | High | [¹¹C]AFM ≈ [¹¹C]DASB > [¹¹C]DAPA ≈ [¹¹C]McN 5652 ≈ [¹¹C]ADAM |
| | | K1 | High | [¹¹C]AFM ≈ [¹¹C]DASB > [¹¹C]ADAM ≈ [¹¹C]McN 5652 ≈ [¹¹C]DAPA |
| | | Minimal scan time | Short | [¹¹C]DASB ≈ [¹¹C]ADAM < [¹¹C]McN 5652 ≈ [¹¹C]AFM ≈ [¹¹C]DAPA |
| | | Non-specific binding | Low | [¹¹C]DASB < [¹¹C]McN 5652 ≈ [¹¹C]ADAM ≈ [¹¹C]DAPA ≈ [¹¹C]AFM |
| | | BP | High | [¹¹C]AFM > [¹¹C]DAPA ≈ [¹¹C]ADAM > [¹¹C] DASB ≈[¹¹C]McN 5652 |
| | | V₃″ | High | [¹¹C]AFM > [¹¹C]DASB ≈ [¹¹C]DAPA > [¹¹C] ADAM ≈[¹¹C]McN 5652 |
| | | Test/retest variability | Low | [¹¹C]AFM ≈ [¹¹C]DAPA < [¹¹C]DASB < [¹¹C]McN 5652 < [¹¹C]ADAM |

^{a} desired property to improve upon the reference radiotracer, [¹¹C] McN 5652.
^{b} tracers are listed in rank order of the parameter.
< or > denote statistically significant differences; ≈ denote statistically insignificant. difference.

In Vitro Evaluation

The in vitro evaluation of affinity yielded values generally comparable to the literature: ADAM and DASB Kᵢ were previously reported as 0.15 nM (Choi et al., 2000) and 1.10 nM (Wilson et al., 2000). The McN 5652 C₅₀ for 5-HT uptake blockade (0.4 nM, Shank et al., 1988)) was also consistent with Kᵢ values measured here. The comparative evaluation performed here indicated that DASB had significantly lower affinity for SERT than the other four compounds, and that this difference was magnified as temperature increases.

The test compounds showed significant differences in lipophilicity, with [¹¹C]DASB and [¹¹C]AFM showing lower lipophilicity than [¹¹C]DASB and [¹¹C]DAPA, with [¹¹C]McN 5652 occupying an intermediate position. It is usually assumed that higher lipophilicity will translate into higher nonspecific binding to plasma proteins and brain membranes (low f₁ and f₂, respectively). This prediction was verified in part here: [¹¹C]DASB and [¹¹C]AFM were less lipophilic than [¹¹C]ADAM and [¹¹C]DAPA, and as a result showed higher f₁ compared to [¹¹C]ADAM and [¹¹C]DAPA. The lipophilicity of [¹¹C]McN 5652 was not significantly different from that of [¹¹C]AFM, and significantly but only slightly higher than that of [¹¹C]DASB. However, [¹¹C]McN 5652 free fraction was not measurable using the conventional ultracentrifugation technique. These observations confirmed that the predictive value of relative lipophilicity measurement for in vivo properties is restricted to compounds belonging to the same chemical structure class.

Integrating affinity and lipophilicity information for the four new compounds, and keeping in mind that low lipophilicity and high affinity are desirable, it appears that AFM provides the best combination of both parameters (FIG. 14). Compared to ADAM and DAPA, the lower lipophilicity of DASB is achieved at the price of a lower affinity. In contrast, the lower lipophilicity of AFM is achieved without a decreased affinity.

Plasma Analysis

Given that [¹¹C]McN 5652 is associated with high but protracted brain uptake, desired properties of new tracers included a faster rate of metabolism and plasma clearance. Everything else being equal, faster clearance from the plasma should result in decreased scanning time. This rule was actually verified by serendipity in this study: baboon A displayed a faster clearance than baboon B (presumably due to its greater weight), but identical SERT availability. Between-baboon differences predictable by this situation were confirmed: compared to baboon B, baboon A displayed lower total brain uptake at 45 minutes, earlier peak time, faster brain wash-out, and higher ROI to cerebellum ratios at 90 minutes. The difference between ROI to cerebellum ratios at 90 minutes between baboon A and B is a good illustration of the dependence of these ratios to the plasma clearance, and of the danger associated with the use of tissue ratios as outcome measure to estimate target site availability (see discussion in Carson et al., 1993). For between-tracer differences, [¹¹C]AFM exhibited a faster metabolism rate, and both [¹¹C]AFM and [¹¹C]DASB exhibited faster plasma clearance.

The plasma free fraction of [¹¹C]McN 5652 is not measurable by the conventional ultracentrifugation method, due to high binding to the ultracentrifugation filters (Parsey et al., 2000). The same situation was encountered with [¹¹C]ADAM and [¹¹C]DAPA. In contrast, filter binding was modest for [¹¹C]AFM and [¹¹C]DASB. [¹¹C]DASB f₁ measured in this study in baboons was similar (13.5±1.7%, n=4) to [¹¹C]DASB f₁ reported previously in humans (11.0±1.2%, n=5) (Ginovart et al., 2001). [¹¹C]AFM f₁ was of comparable magnitude (9.6+0.5%). Both values were lower than the plasma free fraction of [¹²³I]β-CIT (40±5%) (Laruelle et al., 1994a). The ability to measure free fraction in the plasma is critical for clinical studies involving conditions in which this fraction might change, such as alcoholism (Abi-Dargham et al., 1998). Short of f₁ measurement, the significance of differences in brain distribution volumes will be difficult to interpret.

In conclusion, the faster plasma clearance and measurable free fractions of [¹¹C]DASB and [¹¹C]AFM are two factors that favor these two ligands over [¹¹C]McN 5652, [¹¹C] ADAM and [¹¹C]DAPA.

Image Analysis

Simple inspection of images (FIG. 4) and time-activity curves (FIGS. 5-9) reveals major features that differentiate these ligands from one another.

FIG. 5B illustrates the limitations of [¹¹C]McN 5652: the brain uptake was protracted, the regional peak activities were poorly defined, and the differences between cerebellum and ROI activities were modest. The PET images showed a specific accumulation in midbrain and thalamus, while the striatum barely emerges from the neocortical activity, and the hippocampus is not detectable. [11C]ADAM time-activity profile (FIG. 6B) and images were similar to [¹¹C]McN 5652 (with the exception of a relatively higher uptake in the temporal cortex). The uptake of [¹¹C]DASB (FIG. 7B) was very different from [¹¹C]McN 5652, in that it showed a fast uptake, well-defined peaks, and appreciable wash-out. On the [¹¹C]DASB images, the cerebellum was darker, the contrast was better, and the hippocampus was detectable. [¹¹C]DAPA presented a time-activity profile similar to [¹¹C]McN 5652 and [¹¹C]ADAM, with the exception that the differences between regions appeared to be more pronounced (FIG. 8B). The PET images of [¹¹C]DAPA were also similar in term of contrasts to the [α]McN 5652 and [¹¹C]ADAM images. Finally, [¹¹C]AFM shared with [¹¹C]DASB a well defined peak, and appreciable wash-out (FIG. 9B). On the images, [¹¹C]AFM provided a superior contrast, the concentration of activity in midbrain and thalamus was very high, and the striatum and most remarkably the hippocampus clearly emerged from the neocortical background. The quantitative analysis confirmed the impression gained by simple examination of the time-activity curves. Compared to [¹¹C]McN 5652, [¹¹C]ADAM and [¹¹C]DAPA, [¹¹C] DASB exhibited earlier peaks, faster wash-out, and higher ratios at the end of the experiments. [¹¹C]AFM shared with [¹¹C]DASB the higher ratios, but peak activities occurred later than those of [¹¹C]DASB, and less wash-out was observed during the time-frame of the scans.

Uptake Kinetics

Kinetic analysis was performed using a one-tissue compartment model. The better performance of the one-versus the two-tissue compartment model has been previously noted for [¹¹C]-McN 5652 by several groups (Szabo et al., 1999; Buck et al., 2000; Parsey et al., 2000) and for [¹¹C]DASB by Ginovart et al. (2001). Values of K₁ reported in this study were high, indicating excellent extraction and brain penetration. The highest K₁ values displayed by [¹¹C]AFM and [¹¹C]DASB might be related to higher free fraction in the plasma.

Not surprisingly, given its faster kinetic, the average regional minimal scanning time required to derive time-independent estimates of V_{T} was the shortest for [¹¹C]DASB. The midbrain was the region in which a larger data set was required to derive time-independent estimates. In this region, 70 minutes were required for [¹¹C]McN 5652, [¹¹C]ADAM and [¹¹C]AFM. [¹¹C]DASB required only 60 minutes, and [¹¹C]DAPA required 80 minutes.

It is difficult to predict how these values would extrapolate to humans. The rate of metabolism for [¹¹C]McN 5652 is slower in humans than in baboons: at 30 minutes, the [¹¹C]McN 5652 parent fraction was 59±10% in humans (Parsey et al., 2000), compared to 28±13% measured here in baboons. The same was true for [¹¹C]DASB: in humans, [¹¹C]DASB parent fraction at 30 minutes was 40±7% (Ginovart et al., 2001), versus 20±1% measured here. The slower metabolism rate observed in humans might translate into slower brain uptake kinetics. Supporting this prediction, we observed that at least 120 minutes of data were required to derive stable [¹¹C]McN 5652 V_{T} estimates in the human midbrain (Parsey et al., 2000), versus 70 minutes in baboons (50 minutes difference). On the other hand, Ginovart et al. (2001) determined that no more than 80 minutes of data acquisition was required to derive stable V_{T} values in all regions with [¹¹C]DASB: this is only 20 minutes more than in the baboons. A fair conclusion would be that, with the exception of [¹¹C]DASB, the new ligands evaluated here are unlikely to allow for a substantial reduction in scanning time in humans compared to [¹¹C]McN 5652. However, a great uncertainty is associated with this prediction.

Nonspecific Binding

The cerebellum distribution volume was uniformly high for all of these ligands (range of 10 to 30 mL g⁻¹) [¹¹C]McN 5652 V_{T CER} measured in this study (27.7±4.0 mL g⁻¹) was comparable to values previously reported in humans for [¹¹C]McN 5652 in this laboratory (17.8±1.9 mL g⁻¹, n=6) (Parsey et al., 2000) and another (11.9±3.3 mL g⁻¹, n=8) (Buck et al., 2000). [¹¹C]DASB V_{T CER} measured here (17.3±0.5 mL g⁻¹) was also comparable to [¹¹C]DASB V_{T CER} reported in humans (11.8±1.5 mL g⁻¹, n=5) (Ginovart et al., 2001). [¹¹C]ADAM V_{T CER} measured in this study (28.5±4.8 mL g⁻¹) was however much higher than [¹²³I]ADAM V_{T CER} measured previously with SPECT in baboons (2.25±0.46 mL g⁻¹) (Acton et al., 1999a).

V_{T CER} values of these five SERT ligands were about one order of magnitude higher than V_{T CER} values measured for other commonly used PET radioligands such as [¹¹C]raclopride, [¹⁸F]fallypride [¹¹C] WAY 100635 and [¹¹C]NNC 112 (Lammertsma et al., 1996; Abi-Dargham et al., 2000a; Abi-Dargham et al., 2000b; Mawlawi et al., 2001a). However, they are comparable to values measured in humans for the DAT/SERT ligand [¹²³I]β-CIT (28±3 mL g⁻¹) (Laruelle et al., 1994a) and for the 5-HT2A receptor ligand [¹¹C]MDL 100907 (18±4 mL g⁻¹) (Mawlawi et al., 2001b). These high levels of nonspecific distribution volumes represent a major handicap to achieve high signal to noise ratio (V₃″=BP/VTCER). In this regard, the significantly lower V_{T CER} of [¹¹C]DASB compared to the four other tracers provide an appreciable advantage for this ligand.

Receptor Parameters

[¹¹C]AFM had the highest BP of the five ligands tested in this study. The magnitude of BP per se is not critical in defining the imaging qualities of a tracer. BP provides a measure of the signal, but the quality of the signal can only be appreciated in relationship to the noise. The ratio of BP to V_{T CER}, namely V₃″, is thus more related to imaging characteristics than BP itself. The main interest of calculating BP in this context is to relate this value to the Bₘₐₓ/K_{D} ratio. Under the assumption that the tracer crosses the blood brain barrier by passive diffusion (a widely accepted assumption that might not be true for all SSRI, see Rochat et al., 1999), BP as defined here is equal to f₁Bₘₐₓ/K_{D}. The derivation of the Bₘₐₓ/K_{D} ratio requires the knowledge of f₁; and therefore this derivation was only feasible for [¹¹C]DASB and [¹¹C]AFM. Assuming that both ligands bind to the same population of transporter sites, which is a fair assumption, this calculation suggested that the in vivo affinity of [¹¹C]AFM was 3.3 times higher than that of [¹¹C]DASB. This conclusion was consistent with the in vitro data, which indicated a lower Kᵢ for [¹¹C]AFM compared to [¹¹C]DASB, by a factor of 2.8 at room temperature and 4.4 at body temperature Table 3.

As previously stated, V₃″ (=f₂Bₘₐₓ/K_{D}) was a decisive outcome measure of the present study for comparing these SERT ligands. In a given region (Bₘₐₓ being constant), higher V₃″ means higher affinity (lower K_{D}) and/or lower nonspecific binding (higher f₂), improved signal to noise ratio and better measurement reliability. To increase V₃″ above values observed with [¹¹C]McN 5652 might not be critical (or even desirable) to measure SERT in regions of high density, such as the midbrain. However, it is essential for the measurement of SERT availability in regions of lower density, such as the neocortex and the limbic system. The vast majority of postmortem findings implicating alterations of SERT density in psychiatric disorders have been observed in the neocortex and the limbic system. For example, in schizophrenia, abnormalities of SERT density in postmortem studies appear to be localized to the prefrontal cortex and hippocampus, while other areas of the neocortex are preserved (Joyce et al., 1993; Laruelle et al., 1993a; Dean et al., 1995; Gurevich and Joyce, 1997). Using [¹²³I]β-CIT, we observed normal SERT level in the midbrain of patients with schizophrenia (Laruelle et al., 2000a). Thus, schizophrenia is an example of a condition in which a SERT ligand capable of quantifying the SERT concentrations in regions of relatively low density is desirable.

[¹¹C]McN 5652 V₃″ values measured in baboons in this study were consistent with values previously measured in six healthy humans (Parsey et al., 2000), with some noticeable discrepancies, presumably due to species differences: in humans, [¹¹C]McN 5652 V₃″ in the midbrain region was much higher than the thalamus (1.63 versus 0.92, respectively), and this was not the case in baboons (0.94 versus 1.04). Striatal V₃″ was comparable to thalamus V₃″ in humans (1.03 versus 0.92, respectively), but significantly lower in baboons (0.64 versus 1.04). Regarding limbic and cortical regions, the hippocampus, temporal cortex, cingulate cortex and occipital cortex regions showed comparable and low [¹¹C]McN 5652 V₃″ in humans (0.49, 0.22, 0.21 and 0.17, respectively) and baboons (0.34, 0.23, 0.19 and 0.11). Thus, based on [¹¹C]McN 5652 data acquired in the same laboratory, cortical V₃″ measured in baboons appear to be a fair predictor of values observed in humans. On the other hand, [¹¹C]DASB V₃″ reported by Ginovart in the occipital cortex in humans (0.51±0.09) were higher than that measured in baboons in this study (0.11±0.06), using a similar modeling strategy. At this point, it is unclear if this discrepancy reflects species difference or methodological difference in image analysis between laboratories.

Compared to [¹¹C]McN 5652, [¹¹C]ADAM provided a modest improvement in V₃″. Combining all regions, [¹¹C]ADAM V₃″ expressed in percentage of [¹¹C]McN 5652 V₃″ was only 128±24%. [¹¹C]DASB and [¹¹C]DAPA provided more significant V₃″ improvement over [¹¹C]McN 5652 ([¹¹C]DASB and [¹¹C]DAPA V₃″ were 142±29% and 151±18% higher than [¹¹C]McN 5652 V₃″, respectively). Finally, [¹¹C]AFM provided the most significant improvement, with V₃″ values almost twice of that for [¹¹C]McN 5652 (193±35%). These results suggest that, among the ligands tested in this study, [¹¹C]AFM should be the ligand of choice to measure SERT availability in limbic and neocortical regions.

This study did not include examination of the prefrontal cortex. Because of the close proximity of this structure to the striatum in baboons, counts in the prefrontal cortex are contaminated by spill-over from the striatum. In humans, [¹¹C]McN 5652 V₃″ was not significantly different from zero in the dorsolateral prefrontal cortex and the orbitofrontal cortex, and barely above background in the medial prefrontal cortex (V₃″=0.08) (Parsey et al., 2000). This observation does not mean that SERT is absent in these regions, but that the specific binding of the radioligand was negligible. Thus, while data from the present study suggest that [¹¹C]AFM will provide adequate specific signal in hippocampus, temporal, and cingulate cortex, the evaluation of the ability of this tracer to visualize and quantify SERT in the prefrontal cortex will require human studies.

The observation that the relative improvement in V₃″ of the new ligands over [¹¹C]McN 5652 was not uniform across regions was unexpected. Under the assumption that the nonspecific binding and the affinity for SERT of a given ligand are constant across regions, V₃″ should be related to Bₘₐₓ by a region-independent constant. Therefore, if all tracers bind to the same population of transporter sites, relative regional differences in V₃″ should be constant across tracers. To test this prediction, V₃″ values were normalized to the thalamus V₃″ and compared. The expectation for the absence of significant between-tracer differences in normalized V₃″ was generally confirmed (no significant between-tracer differences were observed). However, the presence of a significant tracer by region interaction in this test suggested that in some regions, significant between-tracer differences were encountered in normalized V₃″. In fact, normalized [¹¹C]ADAM and [¹¹C]McN 5652 V₃″ were greater than expected in the striatum, and normalized [¹¹C]ADAM V₃″ was also greater than expected in the limbic and temporal regions (FIG. 13). This observation was reminiscent of the observation reported by Choi et al. (2000) in rats with [¹²³I]ADAM: in the hippocampus, the nonspecific binding, measured by [¹²³I]ADAM accumulation in rats pretreated with 2 mg/kg of unlabelled ADAM, was much higher than other regions. Similarly, in baboons interregional differences in nonspecific binding might affect V₃″ values, especially in regions with low V₃″. Homologous blocking experiments are required to further clarify this issue.

As two experiments were obtained with each ligand in each of the two baboons, an estimate of V₃″ measurement reproducibility could be calculated. These estimates should be taken with caution, as they are derived from only two pairs of observation. Nonetheless, the relative variability between regions and tracers in reproducibility is relatively consistent with what could be predicted from the respective properties of these regions and tracers: thus, regions of large size and greater V₃″ (thalamus and striatum) generally showed a better reproducibility compared to regions of smaller size (midbrain) or V₃″ (cortex). Tracers with higher V₃″ ([¹¹C]AFM and [¹¹C]DAPA) behave better than tracers with lower V₃″ ([¹¹C]McN 5652 and [¹¹C]ADAM), with [¹¹C]DASB occupying an intermediate position.

Overall Evaluation

Examination of Table 14 shows that, for parameters evaluated in vivo, [¹¹C]DASB and [¹¹C]AFM emerge as superior radioligands. Compared to [¹¹C]McN 5652, [¹¹C]ADAM does not appear to provide a clear advantage as a PET imaging agent. However, [¹²³I]ADAM is a very useful and advantageous SPECT radioligand compared to [¹²³I]β-CIT. [¹¹C]DAPA provides some advantage over [¹¹C]McN 5652 in terms of V₃″ and reproducibility, but these improvements are hampered by a relatively slower kinetic and longer required imaging time. [¹¹C]DASB is a superior ligand compared to [¹¹C]McN 5652, as it provides both higher V₃″ and shorter imaging time. For studies in which measurement of limbic and cortical regions are not essential, such as in drug occupancy studies, [¹¹C]DASB is clearly the tracer of choice among the ones tested here. In addition, [¹¹C]DASB has already been evaluated in humans (Houle et al., 2000; Ginovart et al., 2001; Meyer et al., 2001), and the excellent qualities of this tracer observed in animal studies have already been confirmed in humans. On the other hand, [¹¹C]AFM might offer distinct advantages for the measurement of SERT in regions of relatively low densities. [¹¹C]AFM V₃″ was significantly higher than all the other tracers evaluated here. The key factor to determine the usefulness of [¹¹C]AFM in human studies will be the time required to reach time-independent estimate of V_{T}. Another attractive feature of [¹¹C]AFM is that this ligand can be radiolabeled with F-18. The use of [¹⁸F]AFM might make the issue of scan duration less critical, and thus make it possible to take full advantage of the higher V₃″ offered by this ligand. In addition, [¹⁸F]AFM would make SERT imaging available in PET centers without an on-site cyclotron, given that the distribution network for F-18 labeled PET compounds is currently established. For all these reasons, further evaluation of [¹¹C]AFM and [¹⁸F]AFM in humans is warranted.

### D. Conclusion

Four newly developed SERT radioligands were evaluated and compared to the reference tracer [¹¹C]McN 5652 under identical experimental conditions. Overall, [¹¹C]ADAM and [¹¹C]DAPA do not appear to present a clear advantage over [¹¹C]McN 5652 as PET imaging agents. On the other hand, [¹¹C]DASB, because of its fast kinetic, and [¹¹C]AFM, because of its higher signal to noise ratio, appear to be superior radioligands for the imaging SERT in baboons using PET. Studies in humans comparing [¹¹C]McN 5652, [¹¹C]DASB and [¹¹C]AFM are warranted to confirm the results of the present study.

### Experiment 4

### Imaging Study in Baboons with [¹¹C]AFM and [¹⁸F]AFM

One baboon was studied with both [¹¹C]AFM and [¹⁸F]AFM. [¹¹C]AFM was injected first (4.82 mCi, SA of 1240 Ci/mmol, mass of 1.05 μg) and scanned for 90 minutes. The animal was then injected with [¹⁸F]AFM (2.60 mCi, 425 Ci/mmol, 1.51 μg) and scanned for 90 minutes. Analysis of the input function and brain uptake was performed as described in the previous section. Table 15 shows the percent of plasma activity corresponding to the parent compound for each radiotracer. Arterial activity was corrected for parent fraction to form the input function. As expected, the input functions of the parent compound were similar for both tracers, with clearance of 31 and 54 L/hour for [¹¹C] and [¹⁸F] labeled compounds, respectively.

TABLE 15
Percent plasma activity corresponding
to the parent compound (same baboon).

| Time (minutes) | [¹¹C] AFM | [¹⁸F] AFM |
|---|---|---|
| 2 | 75.6% | 63.5% |
| 4 | 55.4% | 40.0% |
| 12 | 21.4% | 21.3% |
| 30 | 9.4% | 8.7% |
| 60 | 6.4% | 5.3% |
| 90 | 6.7% | 4.6% |

The distribution (FIG. 16) and kinetics (FIG. 15) of brain regional uptake of [¹¹C]AFM and [¹⁸F]AFM were also similar. Data were analyzed with a one tissue compartment model. Values of V₃″ (FIG. 15B) were essentially the same for both tracers (for example, thalamus V₃″ was 2.26 for [¹¹C]AFM and 2.27 for [¹⁸F]AFM).

Taken together, results from experiments in both rats and baboon suggest that [¹⁸F]AFM behaves essentially the same as [¹¹C]AFM in vivo. One of the main concerns when evaluating [¹⁸F]AFM in comparison to [¹¹C]AFM was the presence of radiolabeled metabolites in the brain. This concern of significant contamination by radiolabeled metabolite can be excluded on the fact that V₃″ in baboon was similar between both tracers (in case of significant brain penetration of radiolabeled metabolites following [¹⁸F]AFM injection, [¹⁸F]AFM V₃″ should be reduced since “nonspecific binding” would be higher). These data indicated that even though the two tracers might produce different radiolabeled metabolites, these metabolites are not able to cross into the brain and confound the imaging signal.

### Experiment 5

### Further Characterization of [¹⁸F]AFA, [¹⁸F]AFM, and [¹⁸F]AFE in Imaging Studies in Baboons and Comparison with their C-11 Labeled Counterparts

### Imaging Data:

Scans with [¹¹C]AFM, [¹⁸F]AFM, [¹¹C]AFA, [¹⁸F]AFA, [¹¹C]AFE, [¹⁸F]AFA were acquired on one adult male baboon. General scanning methods were as described in Huang et al. (2002). Injected dose and specific activities at time of injection are provided in Table 16. Arterial input function was measured and corrected for metabolites. All radiotracers displayed a rapid clearance (Table 16).

TABLE 16
Imaging data from ¹¹C and ¹⁸F radiolabeled AFM, AFA and AFE.

| | | ID | SA | Clearance | Cerebellum |
|---|---|---|---|---|---|
| Ligand | n | (mCi) | (Ci/mmoles) | (L/h) | V (mL/g) |
| [¹¹C] AFM | 4 | 4.63 ± 0.22 | 1743 ± 1296 | 47 ± 11 | 32 ± 6 |
| [¹⁸F] AFM | 2 | 1.65 ± 1.33 | 446 ± 30 | 40 ± 1 | 23 ± 6 |
| [¹¹C] AFA | 2 | 4.81 ± 1.09 | 2086 ± 1370 | 54 ± 6 | 13 ± 3 |
| [¹⁸F] AFA | 1 | 1.48 | 797.11 | 68 | 24 |
| [¹¹C] AFE | 1 | 5.08 | 949.00 | 73 | 16 |
| [¹⁸F] AFE | 1 | 1.68 | 1447.97 | 71 | 19 |

Activity distributed in the brain in accordance to the known density distribution pattern of SERT (Laruelle et al., 1988). Highest uptake was noted in midbrain, thalamus and striatum. Data were analyzed with a two-compartment model, using the metabolite-corrected arterial time-activity curve as input function. This analysis provided the regional distribution volumes (V_{T}, mL/g).

The nonspecific distribution volume, measured as the distribution volume of the cerebellum (a region with negligible SERT density) was in a similar range for all tracers (10 to 30 mL/g, Table 16).

The specific to nonspecific partition coefficient, V₃″, was calculated as the ratio of V_{T} in the region of interest (ROI) to cerebellum V_{T} minus one. V₃″ provides a measure related to receptor parameters as follows:

$${V}_{3}^{″} = \frac{{B}_{max}}{{V}_{2} {K}_{D}}$$

where Bₘₐₓ is the maximal density of sites, V₂ is the nonspecific distribution volume (equal to cerebellum V_{T}) and K_{D} is the in vivo affinity of the radiotracer for the target. V₃″ in midbrain, thalamus, striatum, cingulate cortex, temporal cortex, parietal cortex, and occipital cortex are presented in FIG. 17. The parameter V₃″ is the most important parameter to evaluate the suitability of a tracer to image the target of interest, as it is directly related to the signal to noise contrast.

Regional variations in V₃″ for all tracer evaluated were consistent with regional variations in SERT density. Two observations appear to be supported by these data:
1. For all tracers, V₃″ measured with ¹¹C labeled compound and ¹⁸F labeled compound were similar.
2. Regional V₃″ of [¹¹C]/[¹⁸F]AFM were higher than those of [¹¹C]/[¹⁸F]AFA and [¹¹C]/[¹⁸F]AFE, presumably due to the higher affinity of AFM for SERT compared to the other two compounds.

In conclusion, all radiotracers appear to be suitable for imaging the SERT in the living brain. Among the radioligands evaluated, [¹¹C]/[¹⁸F]AFM provides the highest signal to noise ratios and V₃″ in all brain regions. Development of [¹⁸F]AFM and related radioligands should allow the emergence of PET radioligands that possess the dual features of superior in vivo imaging properties and feasibility for wide distribution and applications.

### REFERENCES

1. Goodman, M. M., et al., Halogenated Naphthyl Methoxy Piperidines for Mapping Serotonin Transporter Sites. U.S. Pat. No. 5,919,797, issued Jul. 6, 1999.
2. The Wellcome Foundation, Mehta, N. B. et al., Halogen Substituted Diphenylsulfides. European Patent Application No. 90 30 60 97.8, filed Jun. 5, 1990.
3. Polivka, Z. et al., Derivatives of N,N-Dimethyl-2-(Arylthio)Benzylamine, Their Salts, Methods of Preparation and Their Use in Pharmaceutical Medicaments, International Application No. PCT/CZ96/00022, filed Nov. 7, 1996.
4. Abi-Dargham A., Laruelle M., Seibyl J., et al. (1994) SPECT measurement of benzodiazepine receptors in human brain with [¹²³-I]iomazenil: kinetic and equilibrium paradigms. J. Nucl. Med. 35:228-238.
5. Abi-Dargham A., Krystal J. H., Anjilvel S., et al. (1998) Alterations of benzodiazepine receptors in type II alcoholic subjects measured with SPECT and [I-123]iomazenil. Am. J. Psychiatry 155:1550-1555.
6. Abi-Dargham A., Simpson N., Kegeles L., et al. (1999) PET studies of binding competition between endogenous dopamine and the D1 radiotracer [¹¹C]NNC 756. Synapse 32:93-109.
7. Abi-Dargham A., Hwang D. R., Huang Y., et al. (2000a) Reliable quantification of both striatal and extrastriatal D2 receptors in humans with [¹⁸F]fallypride. J. Nucl. Med. 41:139P.
8. Abi-Dargham A., Martinez D., Mawlawi O., et al. (2000b) Measurement of striatal and extrastriatal dopamine D1 receptor binding potential with [¹¹C]NNC 112 in humans: validation and reproducibility. J. Cereb. Blood Flow Metab. 20:225-243.
9. Acton P. D., Kung M. P., Mu M., et al. (1999a) Single-photon emission tomography imaging of serotonin transporters in the non-human primate brain with the selective radioligand [(¹²³)I]IDAM. Eur. J. Nucl. Med. 26:854-861.
10. Acton P. D., Mu M., Plossl K., et al. (1999b) Single-photon emission tomography imaging of serotonin transporters in the nonhuman primate brain with [(¹²³)I]ODAM. Eur. J. Nucl. Med. 26:1359-1362.
11. Acton P. D., Choi S. R., Hou C., et al. (2001) Quantification of serotonin transporters in nonhuman primates using [(¹²³)I]ADAM and SPECT. J. Nucl. Med. 42:1556-1562.
12. Backstrom I., Bergstrom M., Marcusson J. (1989) High affinity [³H]paroxetine binding to serotonin uptake sites in human brain tissue. Brain Res. 486:261-268.
13. Bergstrom K. A., Halldin C., Hall H., et al. (1997) In vitro and in vive characterisation of nor-beta-CIT: A potential radioligand for visualisation of the serotonin transporter in the brain. Eur. J. Nucl. Med. 24:596-601.
14. Brix G., Zaers J., Adam L. E., et al. (1997) Performance evaluation of a whole-body PET scanner using the NEMA protocol. National Electrical Manufacturers Association. J. Nucl. Med. 38:1614-1623.
15. Buck A., Gucker P. M., Schonbachler R. D., et al. (2000) Evaluation of serotonergic transporters using PET and [¹¹C](+)McN-5652: assessment of methods. J. Cereb. Blood Flow Metab. 20:253-262.
16. Carson R. E., Channing M. A., Blasberg R. G., et al. (1993) Comparison of bolus and infusion methods for receptor quantitation: application to [¹⁸F]cyclofoxy and positron emission tomography. J. Cereb. Blood Flow Metab. 13:24-42.
17. Choi S. R., Hou C., Oya S., et al. (2000) Selective in vitro and in vivo binding of [(¹²⁵)I]ADAM to serotonin transporters in rat brain. Synapse 38:403-412.
18. Cortes R., Soriano E., Pazos A., et al. (1988) Autoradiography of antidepressant binding sites in the human brain: localization using [³H]imipramine and [³H]paroxetine. Neuroscience 27:473-496.
19. Dannals R. F., Ravert H. T., Wilson A. A., Wagner H. N., Jr. (1990) Synthesis of a selective serotonin uptake inhibitor: [¹¹C]citalopram. Int. J. Rad. Appl. Instrum. [A] 41:541-543.
20. Dean B., Opeskin K., Pavey G., et al. (1995) Copolov DL (1995) [³H]paroxetine binding is altered in the hippocampus but not the frontal cortex or caudate nucleus from subjects with schizophrenia. J. Neurochem. 64:1197-1202.
21. Gandelman M. S., Baldwin R. M., Zoghbi S. S., et al. (1994) Evaluation of ultrafiltration for the free fraction determination of single photon emission computerized tomography (SPECT) radiotracers: b-CIT, IBF and iomazenil. J. Pharmaceutical Sci. 83:1014-1019.
22. Ginovart N., Wilson A. A., Meyer J. H., et al. (2001) Positron Emission Tomography Quantification of [¹¹C]-DASB Binding to the Human Serotonin Transporter: Modeling Strategies. J. Cereb. Blood Flow Metab. 21:1342-1353.
23. Gurevich E. V., Joyce J. N. (1997) Alterations in the cortical serotonergic system in schizophrenia: A postmortem study. Biol. Psychiatry 42:529-545.
24. Hashimoto K., Inoue O., Suzuki K., et al. (1987) Synthesis and evaluation of [¹¹C]cyanoimipramine. Int. J. Rad. Appl. Instrum. [B] 14:587-592.
25. Heinz A., Ragan P., Jones D. W., et al. (1998) Reduced central serotonin transporters in alcoholism. Am. J. Psychiatry 155:1544-1549.
26. Houle S., Ginovart N., Hussey D., et al. (2000) Imaging the serotonin transporter with positron emission tomography: initial human studies with [¹¹C]DAPP and [¹¹C]DASB. Eur. J. Nucl. Med. 27:1719-1722.
27. Huang Y. Y., Mahmood K., Simpson N. R., et al. (1998) Stereoconservative synthesis of the enantiomerically pure precursors of [C-11](+)-McN 5652 and [C-11](−)-McN 5652. J. Label. Compound Radiopharm. 41:9-17.
28. Huang Y., Bae S. A., Zhu Z., et al. (2001a) Fluorinated analogues of ADAM as new PET radioligands for the serotonin transporter: synthesis and pharmacological evaluation. J. Labelled Compd. Radiopharm. 44:S18-S20.
29. Huang Y., Bae S. A., Zhu Z., et al. (2001b) Synthesis and Characterization of a New PET Ligand for the Serotonin Transporter: [¹¹C]₅-Bromo-2-{2-[(dimethylamino)methyl]phenyl sulfanyl}phenylamine. J. Nuc. Medicine 42.
30. Huang Y., Hwang D. R., Narendran R., Sudo Y., et al. (2002) Comparative Evaluation in Nonhuman Primates of Five PET Radiotracers for Imaging the Serotonin Transporters: [¹¹C]McN 5652, [¹¹C]ADAM, [¹¹C]DASB, [¹¹C]DAPA, and [¹¹C]AFM. J. Cereb. Blood Flow Metab. 22:1377-1398.
31. Hume S. P., Pascali C., Pike V. W., et al. (1991) Citalopram: labelling with carbon-11 and evaluation in rat as a potential radioligand for in vivo PET studies of 5-HT re-uptake sites. Int. J. Rad. Appl. Instrum. [B] 18:339-351.
32. Jacobsen L. K., Staley J. K., Malison R. T., et al. (2000) Elevated central serotonin transporter binding availability in acutely abstinent cocaine-dependent patients. Am. J. Psychiatry 157:1134-1140.
33. Jagust W. J., Eberling J. L., Biegon A., et al. (1996) Iodine-123-5-iodo-6-nitroquipazine: SPECT radiotracer to image the serotonin transporter. J. Nucl. Med. 37:1207-1214.
34. Joyce J. N., Shane A., Lexow N., et al. (1993) Serotonin uptake sites and serotonin receptors are altered in the limbic system of schizophrenics. Neuropsychopharmacology 8:315-336.
35. Kung M. P., Hou C., Oya S., et al. (1999) Characterization of [(¹²³)I]IDAM as a novel single-photon emission tomography tracer for serotonin transporters. Eur. J. Nucl. Med. 26:844-853.
36. Lammertsma A. A., Bench C. J., Hume S. P., et al. (1996) Comparison of methods for analysis of clinical [¹¹C]raclopride studies. J. Cereb. Blood Flow Metab. 16:42-52.
37. Laruelle M., Vanisberg M. A., Maloteaux J. M. (1988) Regional and subcellular localization in human brain of [³H]paroxetine binding, a marker of serotonin uptake sites. Biol. Psychiatry 24:299-309.
38. Laruelle M., Abi-Dargham A., Casanova M. F., et al. (1993a) Selective abnormalities of prefrontal serotonergic receptors in schizophrenia. A postmortem study. Arch. Gen. Psychiatry 50:810-818.
39. Laruelle M., Baldwin R. M., Malison R. T., et al. (1993b) SPECT imaging of dopamine and serotonin transporters with [¹²³I]b-CIT: pharmacological characterization of brain uptake in nonhuman primates. Synapse 13:295-309.
40. Laruelle M., Wallace E., Seibyl J. P., et al. (1994a) Graphical, kinetic and equilibrium analysis of [¹²³I]b-CIT in vivo binding to dopamine transporters in healthy subjects. J. Cereb. Blood Flow Metab. 14:982-994.
41. Laruelle M., van Dyck C., Abi-Dargham A., et al. (1994b) Compartmental modeling of iodine-123-iodobenzofuran binding to dopamine D₂ receptors in healthy subjects. J. Nucl. Med. 35:743-754.
42. Laruelle M., Baldwin R. M., Rattner Z., et al. (1994c) SPECT quantification of [¹²³I]iomazenil binding to benzodiazepine receptors in nonhuman primates. I. Kinetic modeling of single bolus experiments. J. Cereb. Blood Flow Metab. 14:439-452.
43. Laruelle M., Abi-Dargham A., van Dyck C., et al. (2000) Dopamine and serotonin transporters in patients with schizophrenia: an imaging study with [(¹²³) I]beta-CIT. Biol. Psychiatry 47:371-379.
44. Lasne M. C., Pike V. W., Turton D. R. (1989) The radiosynthesis of [N-methyl-¹¹C]-sertraline. Int. J. Rad. Appl. Instrum. [A] 40:147-151.
45. Livni E., Satterlee W., Robey R. L., et al. (1994) Synthesis of [¹¹C]dapoxetine.HCl, a serotonin re-uptake inhibitor: biodistribution in rat and preliminary PET imaging in the monkey. Nucl. Med. Biol. 21:669-675.
46. Lundkvist C., Loc'h C., Halldin C., et al. (1999) Characterization of bromine-76-labelled 5-bromo-6-nitroquipazine for PET studies of the serotonin transporter. Nucl. Med. Biol. 26:501-507.
47. Malison R. T., Price L. H., Berman R., et al. (1998) Reduced brain serotonin transporter availability in major depression as measured by [¹²³I]-2 beta-carbomethoxy-3 beta-(4-iodophenyl)tropane and single photon emission computed tomography. Biol. Psychiatry 44:1090-1098.
48. Mann, J. J., et al. (2000) A serotonin transporter gene promoter polymorphism (5-HTTLPR) and prefrontal cortical binding in major depression and suicide. Arch. Gen. Psychiatry 57:729-738.
49. Mawlawi O., Martinez D., Slifstein M., et al. (2001a) Imaging human mesolimbic dopamine transmission with positron emission tomography: I. Accuracy and precision of D2 receptor parameter measurements in ventral striatum. J. Cereb. Blood Flow Metab. 21:1034-1057.
50. Mawlawi O., Huang Y., Hwang D., et al. (2001b) Mapping the 5HT2A receptors in human brain with ¹¹CMDL 100907: validation and reproducibility. J. Nuc. Med. 42.
51. McCann U. D., Szabo Z., Scheffel U., et al. (1998). Positron emission tomographic evidence of toxic effect of MDMA (“Ecstasy”) on brain serotonin neurons in human beings. Lancet 352:1433-1437.
52. Meyer J. H., Wilson A. A., Ginovart N., et al. (2001) Occupancy of serotonin transporters by paroxetine and citalopram during treatment of depression: a [(¹¹)C]DASB PET imaging study. Am. J. Psychiatry 158:1843-1849.
53. Mintun M. A., Raichle M. E., Kilbourn M. R., et al. (1984) A quantitative model for the in vivo assessment of drug binding sites with positron emission tomography. Ann. Neurol. 15:217-227.
54. Nelson D. R., Palmer K. J., Johnson A. M. (1991) The monoamine uptake inhibitory properties of Todo-6′-paroxetine. Biol. Psychiatry 29:632 S.
55. Neumeyer J. L., Wang S., Milius R. A., et al. (1991) [¹²³I]2β-carbomethoxy-3β-(4-iodophenyl)tropane (β-CIT): high affinity SPECT radiotracer of monoamine reuptake sites in brain. J. Med. Chem. 34:3144-3146.
56. Oya S., Kung M. P., Acton P. D., et al. (1999) A new single-photon emission computed tomography imaging agent for serotonin transporters: [¹²³I]IDAM, 5-iodo-2-((2-((dimethylamino)methyl)phenyl)thio)benzyl alcohol. J. Med. Chem. 42:333-335.
57. Oya S., Choi S., Hou C., et al. (2000) 2-((2-((dimethylamino)methyl)phenyl)thio)-5-iodophenylamine (ADAM): an improved serotonin transporter ligand. Nucl. Med. Biol. 27:249-254.
58. Parsey R. V., Kegeles L. S., Hwang D. R., et al. (2000) In vivo quantification of brain serotonin transporters in humans using [¹¹C]McN 565 J. Nucl. Med. 41:1465-1477.
59. Plenge P., Mellerup E. T., Laursen H. (1990) Regional distribution of the serotonin transport complex in human brain, identified with ³H-paroxetine, ³H-citalopram and ³H-imipramine. Prog. Neuropsychopharmacol Biol. Psychiatry 14:61-72.
60. Rochat B., Baumann P., Audus K. L. (1999) Transport mechanisms for the antidepressant citalopram in brain microvessel endothelium. Brain Res. 831:229-236.
61. Rosel P., Menchon J. M., Oros M., et al. (1997) Regional distribution of specific high affinity binding sites for ³H-imipramine and ³H-paroxetine in human brain. Neural. Transm. 104:89-96.
62. Rowland M., Tozer T. N. (1989) Clinical Pharmacokinetics, second edition. Philadelphia, Pa.: Lea & Febiger.
63. Scheffel U., Dannals R. F., Suchiro M., et al. (1994) Development of PET/SPECT ligands for the serotonin transporter. Nida. Res. Monogr. 138:111-130.
64. Shank R. P., Vaught J. L., Pelley K. A., et al. (1988) McN-5652: a highly potent inhibitor of serotonin uptake. J. Pharmacol. Exp. Ther. 247:1032-1038.
65. Smith D. F., Jensen P. N., Gee A. D., et al. (1997) PET neuroimaging with [¹¹C]venlafaxine: serotonin uptake inhibition, biodistribution and binding in living pig brain. Eur. Neuropsychopharmacol 7:195-200.
66. Suchiro, M. et al. (1991) Radiosynthesis and evaluation of N-(3-[¹⁸F]fluoropropyl)paroxetine as a radiotracer for in vivo labeling of serotonin uptake sites by PET. Int J Rad Appl Instrum B 18: 791-796.
67. Suchiro M., Scheffel U., Ravert H. T., et al. (1993a) [¹¹C](+)McN5652 as a radiotracer for imaging serotonin uptake sites with PET. Life Sci. 53:883-892.
68. Suchiro M., Scheffel U., Dannals R. F., et al. (1993b) A PET radiotracer for studying serotonin uptake sites: carbon-11-McN-5652Z. J. Nucl. Med. 34:120-127.
69. Szabo Z., Scheffel U., Suchiro M., et al. (1995a) Positron emission tomography of 5-HT transporter sites in the baboon brain with [¹¹C]McN5652. J. Cereb. Blood Flow Metab. 15:798-805.
70. Szabo Z., Kao P. F., Scheffel U., et al. (1995b) Positron emission tomography imaging of serotonin transporters in the human brain using [¹¹C](+)McN5652. Synapse 20:37-43.
71. Szabo Z., Scheffel U., Mathews W. B., et al. (1999) Kinetic analysis of [¹¹C]McN5652: a serotonin transporter radioligand. J. Cereb. Blood Flow Metab. 19:967-981.
72. Tarkiainen, J., Vercouillie, J., Emond, P., et al. (2001) Carbon-11 labelling of MADAM in two different positions: a highly selective PET radioligand for the serotonin transporter. J. Labelled Cpd. Radiopharm. 44, 1013-1023.
73. Tiihonen J., Kuikka J T., Bergstrom K. A., et al. (1997) Single-photon emission tomography imaging of monoamine transporters in impulsive violent behaviour. Eur. J. Nucl. Med. 24:1253-1260.
74. Vercouillie J., Tarkiainen J., Halldin C., et al. (2001) Precursor synthesis and radiolabeling of [¹¹C]ADAM: a potent radioligand for the serotonin transporter exploration by PET. J. Labelled Cpd. Radiopharm. 44:113-120.
75. Willeit M., Praschak-Rieder N., Neumeister A., et al. (2000) [¹²³I]beta-CIT SPECT imaging shows reduced brain serotonin transporter availability in drug-free depressed patients with seasonal affective disorder. Biological Psychiatry 47:482-489.
76. Wilson A. A., Houle S. (1999) Radiosynthesis of ¹¹C-labelled 2-(arylthio)benzylamines: Potential radiotracers for the serotonin reuptake receptpr. J. Labelled Cpd. Radiopharm. 42:1277-88.
77. Wilson A. A., Ginovart N., Schmidt M., et al. (2000) Novel radiotracers for imaging the serotonin transporter by positron emission tomography: synthesis, radiosynthesis, and in vitro and ex vivo evaluation of (¹¹)C-labeled 2-(phenylthio)araalkylamines. J. Med. Chem. 43:3103-3110.
78. Wilson A. A., Jin L., Garcia A., et al. (2001). An admonition when measuring the lipophilicity of radiotracers using counting techniques. Appl. Radiat. Isot. 54:203-208.
79. Woods R. P., Mazziotta J. C., Cherry S. R. (1993) MRI-PET registration with automated algorithm. J. Comput. Assist. Tomogr. 17:536-546.
80. Zea-Ponce Y., Baldwin R. M., Stratton M. D., et al. (1997) Radiosynthesis and PET imaging of [N-methyl-¹¹C]LY257327 as a tracer for 5-HT transporters. Nucl. Med. Biol. 24:251-254.
81. Zhuang Z., Choi S., Hou C., et al. (2000) A novel serotonin transporter ligand: (5-iodo-2-(2-dimethylaminomethylphenoxy)-benzyl alcohol. Nucl. Med. Biol. 27:169-175.

What is claimed:

1. A compound having the structure: 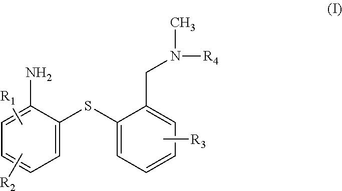
or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, (CH₂)ₙCH₃, or (CH₂)ₙCH₂X, where n=0, 1, 2, or 3;
wherein only one of R₁, R₂, and R₃ is H;
wherein X=halogen;
wherein if n=0 then (CH₂)ₙ is absent;
wherein a carbon atom in R₄ may be [¹¹C]; and
wherein any halogen in the compound may be a radioisotope.

2. The compound of claim 1, wherein one carbon in R₄ is [¹¹C].

3. The compound of claim 1, wherein one halogen in the compound is a radioisotope.

4. The compound of claim 3, wherein the radioisotope is [¹⁸F].

5. The compound of claim 4, wherein the halogen in R₁ is [¹⁸F].

6. The compound of claim 5, wherein R₁ is [¹⁸F].

7. The compound of claim 1, wherein R₁ is F.

8. The compound of claim 1, wherein R₂ is CH₃.

9. The compound having the structure: 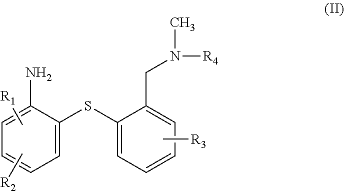
or a physiologically acceptable salt thereof;
wherein R₁ is X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, or (CH₂)ₙCH₃, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope.

10. The compound of claim 9 wherein the compound is 2-(2-dimethylaminomethylphenylthio)-4-fluoro-5-methylphenyl amine.

11. A compound having the structure: 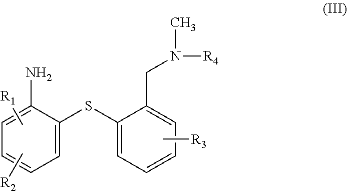
or a physiologically acceptable salt thereof;
wherein R₁ is F, (CH₂)ₙF, ¹⁸F, or (CH₂)ₙ¹⁸F, where n=1, 2, 3, or 4;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₁ is H, or (CH₂)ₙCH₃, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope.

12. The compound of claim 11 having the structure: 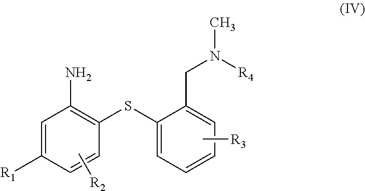
or a physiologically acceptable salt thereof;
wherein R₁ is F, (CH₂)ₙF, ¹⁸F, or (CH₂)ₙ¹⁸F, where n=1, 2, 3, or 4;
wherein R₂ is H;
wherein R₃ is H;
wherein R₄ is H, (CH₂)ₙCH₃, (CH₂)ₙCH₂X, (CH₂)ₙ¹¹CH₃, or (CH₂)ₙ¹¹CH₂X where n=0, 1, 2, or 3,
wherein X=halogen; and
wherein if n=0 then (CH₂)ₙ is absent.

13. The compound of claim 11, wherein R₂ is H, F, Br, (CH₂)ₙCH₃ or (CH₂)ₙCH₂X.

14. The compound of claim 12 wherein one carbon in R₄ is [¹¹C].

15. The compound of claim 12 wherein R₁ is ¹⁸F and R₄ is CH₃.

16. The compound of claim 12 wherein R₁ is CH₂¹⁸F, and R₄ is CH₃.

17. The compound of claim 12 wherein R₁ is (CH₂)₂¹⁸F and R₄ is CH₃.

18. The compound of claim 12 wherein R₁ is (CH₂)₃¹⁸ and R₄ is CH₃.

19. The compound of claim 12 wherein R₁ is (CH₂)₄¹⁸F and R₄ is CH₃.

20. The compound of claim 12 wherein R₁ is ¹⁸F and R₄ is H.

21. The compound of claim 12 wherein R₁ is (CH₃)¹⁸F and R₄ is H.

22. The compound of claim 12 wherein R₁ is (CH₂)₂¹⁸F and R₄ is H.

23. The compound of claim 12 wherein R₁ is (CH₂)₃¹⁸F and R₄ is H.

24. The compound of claim 12 wherein R₁ is (CH₂)₄¹⁸F and R₄ is H.

25. The compound of claim 12 wherein R₁ is (CH₂)ₙF where n=0, 1, 2, 3, or 4 and R₄ is ¹¹CH₃.

26. The compound of claim 12 wherein R₁ is F and R₄ is ¹¹CH₃.

27. The compound of claim 12 wherein R₁ is CH₂F and R₄ is ¹¹CH₃

28. The compound of claim 12 wherein R₁ is (CH₂)₂F and R₄ is ¹¹CH₃.

29. The compound of claim 12 wherein R₁ is (CH₂)₃F and R₄ is ¹¹CH₃.

30. The compound of claim 12 wherein R₁ is (CH₂)₄F and R₄ is ¹¹CH₃.

31. The compound of claim 12 wherein the compound is 2-((2-((dimethylamino)methyl)phenyl)thio)-5-Fluoro-phenylamine.

32. The compound of claim 12 wherein the compound is 5-Fluoro-2-((2-((methylamino)methyl)phenyl)thio)phenylamine.

33. The compound of claim 12 wherein the compound is 2-{2-[(dimethylamino)methyl]phenylthio}-5-fluoromethyl phenylamine.

34. The compound of claim 12 wherein the compound is 2-(2-dimethylaminomethyl-phenylthio)-5-fluoroethylphenylamine.

35. The compound of claim 12 wherein the compound is 2-(2-dimethylaminomethyl-phenylthio)-5-fluoropropylphenylamine.

36. A physiologically acceptable composition comprising the compound of claim 1, 9, 11 or 12 and a physiologically acceptable carrier.

37. A process of making a physiologically acceptable composition comprising mixing the compound of claim 1, 9, 11 or 12 with a physiologically acceptable carrier.

38. A process for synthesizing a compound having the structure: 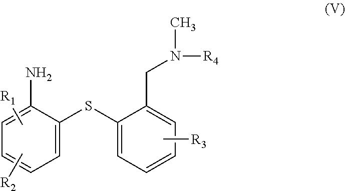
or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙ(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, or (CH₂)ₙCH₃, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope,
comprising
a) reacting a compound having the structure 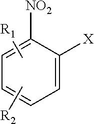
with a compound having the structure 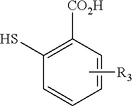
so as to form a product compound having the structure: 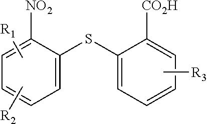
b) reacting the product compound of step a) with CH₃NHR₄ or its salt to form a product compound having the structure: 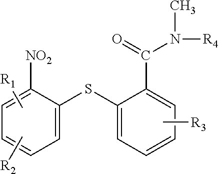
c) reacting the product compound of step b) with a reducing agent to form a product compound having the structure:

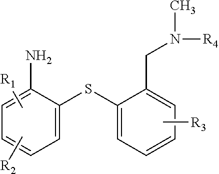

39. A process for radiolabeling with [¹¹C] a compound having the structure: 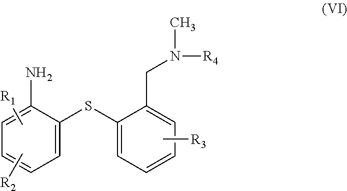
or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)ₙO—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein X=halogen;
wherein if n=0 then (CH₂)ₙ is absent;
wherein a carbon atom in R₄ may be [¹¹C]; and
wherein any halogen in the compound may be a radioisotope,
comprising reacting the compound with [¹¹C]CH₃I in the presence of a suitable solvent.

40. The process of claim 39, wherein the suitable solvent is N,N-dimethylformamide (DMF) or acetone.

41. A process for radiolabeling with [¹⁸F] a compound having the structure: 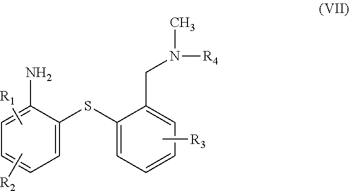
or a physiologically acceptable salt thereof;
wherein R₁ is H, X, CH₃(CH₂)ₙO—, X(CH₂)ₙO—, CH₃(CH₂)ₙS—, X(CH₂)ₙS—, CH₃(CH₂)ₙOCH₂—, X(CH₂)ₙOCH₂—, CH₃(CH₂)ₙ(O)—, X(CH₂)ₙ(O)—, CH₃(CH₂)(O)O—, X(CH₂)ₙ(O)O—, (CH₂)ₙCH₃, or (CH₂)ₙCH₂X, where n=0, 1, 2, or 3;
wherein R₂ is H, X, (CH₂)ₙCH₃, or (CH₂)ₙCH₂X, where n=0, 1, 2, or 3;
wherein R₃ is H, X, (CH₂)ₙCH₃, or (CH₂)ₙCH₂X, where n=0, 1, 2, or 3;
wherein R₄ is H, (CH₂)ₙCH₃, or (CH₂)ₙCH₂X, where n=0, 1, 2, or 3;
wherein only one of R₁, R₂, and R₃ is H;
wherein X=halogen;
wherein if n=0 then (CH₂)ₙ is absent; and
wherein any halogen in the compound may be a radioisotope,
comprising reacting the compound with a salt of ¹⁸F⁻.

42. The process of claim 41, wherein the salt is K¹⁸F.

43. A non-invasive method for positron emission tomography (PET) imaging of serotonin transporter sites in a mammal comprising labeling serotonin transporter sites (SERT) with an image-generating amount of the radiolabeled compound of claim 1, 9, 11 or 12 and measuring spatial distribution of the compound in the mammal by PET so as to thereby image the serotonin transporter sites.
